(19) United States
(12) Patent Application Publication
(10) Pub. No.: US 2012/0231037 A1
(43) Pub. Date: Sep. 13, 2012

(54) CRYSTALLINE DRUG-CONTAINING COATINGS

(75) Inventors: Yair Levi, Haifa (IL); Abraham Jackob Domb, Efrat (IL); Nir Amir, Kiryat-Ono (IL); Nino Eliyahu, Jerusalem (IL); Uri Cohn, Bat-Yam (IL); Noam Tal, Rishon-LeZion (IL)

(73) Assignee: Yissum Research Development Companyof the Hebrew U, Jerusalem (IL)

(21) Appl. No.: 13/147,384

(22) PCT Filed: Feb. 1, 2010
(86) PCT No.: PCT/IL10/00086

§ 371 Date: Apr. 23, 2012

Related U.S. Application Data
(60) Provisional application No. 61/202,163, filed on Feb. 2, 2009.

(51) Int. Cl. A61K 9/00; A61P 7/02; A61P 3/06; A61P 31/04; A61P 29/00; A61P 3/00; A61P 9/00; A61P 35/00; A61P 39/06; A61P 25/00; A61P 37/00; A61P 9/10; A61P 7/00; A61P 19/00; A61P 31/00; A61P 37/06; A61P 1/00; A61P 1/16; A61P 3/02; B05D 3/00; B05C 9/12; A61K 31/436

(52) U.S. Cl. 424/400; 514/291; 427/2.1; 118/69

(57) ABSTRACT

Articles-of-manufacturing comprising an object having a surface and a therapeutically active agent being deposited onto at least a portion of the surface, while at least a portion of said therapeutically active agent being in a crystalline form thereof are disclosed. Methods utilizing such articles-of-manufacturing for treating medical conditions are also disclosed. Processes of preparing the articles-of-manufacturing by contacting a surface of the object with a solution containing the therapeutically active agent; and cooling the surface to a temperature below a temperature of the solution, and apparatus for performing these processes, are also disclosed.

### FIELD AND BACKGROUND OF THE INVENTION

The present invention, in some embodiments thereof, relates to surfaces having applied thereon therapeutically active agents and, more particularly, but not exclusively, to articles-of-manufacturing such as medical devices having applied thereon a crystalline form of a therapeutically active agent.

Crystallization has been the most important separation and purification process in the pharmaceutical industry throughout its history. Yet, crystallization is also of utmost importance in many other fields such as inorganic chemistry, protein chemistry and plating.

Crystallization is a complex process that comprises primarily a phase change from liquid to solid. This change is accompanied by a decrease of entropy as a result of formation of a highly organized crystalline structure. Nucleation and growth are the two dominant processes in a crystallization process and usually occur simultaneously. Controlling a crystallization procedure therefore requires control of both these parameters.

Nucleation has been long considered as the primer process. However, as nucleation depends on the molecular structure of the substrate on which crystallization occurs, it is difficult to control this process.

On the other hand, growth depends to a larger extent on the physical conditions, such as temperature, degree of supersaturation, etc., under which the crystallization is effected.

The different parameters that affect the kinetics of crystallization have been thoroughly studied hitherto. See, for example, Shekunov & York, J. Crystal Growth, 2000, 211:122-136; Li et al., J. Crystal Growth, 2007, 304:219-224; Hartman, American Mineralogist, 1977, 62:1034-1035; Piana et al., Nature, 2005, 438:70-73; and Glicksman & Lupulesco, J. Crystal Growth, 2004, 264:541-549.

Crystallization is an important feature in the pharmaceutical industry, due to the need to meet regulations, and further, because of the significant effect of the crystalline structure on different physical properties, such as stability, bioavailability and dissolution [Li et al., J. Crystal Growth, 2007, 304:219-224] of a pharmaceutically active agent (a drug). The effect of polymorphic and crystalline forms on dissolution rate and/or oral bioavailability of several pharmaceutically active agents have been widely studied [Blagden et al., Advanced Drug Delivery Reviews, 2007, 59:617-630; Morris et al., Advanced Drug Delivery Reviews, 2001, 48:91-114; Fokkens & De Blaey, J. Pharmacy World & Science, 1982, 4:117-121; Agafonov et al., J. Pharm. Sci., 1991, 80:181-185; and Nokhodci et al., J. Crystal Growth, 2005, 274:573-584]. In most cases, the amorphous phase is of higher energy than the crystalline phase and therefore has been used for increasing by order of magnitude dissolution and absorption of a drug.

Crystal engineering offers several routes for improving solubility and dissolution rate of pharmaceutically active agents, which can be adopted through an in-depth knowledge of crystallization processes and the molecular properties of the agent [Paul et al., Powder Technology, 2005, 150:133-143].

Solubility, dissolution rate and other properties are known to affect a performance of drug-loaded implantable medical devices such as drug-eluting stents (DES).

Drug-eluting stents (DES) are frequently used in the treatment of coronary artery disease given their anti-restenotic effect. Currently available DESs are stents coated with anti-proliferative agents that reduce or prevent inflammation and exaggerated SMCs proliferation and accumulation, and thereby reduce restenosis. Examples of such drug eluting stents are paclitaxel-eluting stent (TAXUS®, Boston Scientific), which inhibits the proliferation of SMCs, and sirolimus (rapamycin)-eluting stent (Cypher®, Cordis Corporation), which inhibits the inflammation response of the arterial wall.

In these DESs, a polymeric carrier is used for loading the anti-proliferative agent onto the stent. Unfortunately, the presently commercially available DES systems use polymers which are at least partially biostable, namely, remain stable and non-degradable under in-vivo conditions.

Being is direct contact with the blood and surrounding tissues, the biostable polymers used as drug carrier vehicles in DESs adversely affect/promote several medical conditions and processes is DES, most commonly in-stent thrombosis. Consequently, DES patients are usually treated with anti-platelet therapy for a prolonged time period, which is also associated with adverse side effects and complications.

Additional disadvantages affected by biostable polymeric carriers include inflammation, an incomplete release of the loaded drug (drug entrapment), a potential for permanent damage during delivery and implantation, an increased incidence of thrombus formation, distal embolization, a delayed or abnormal endothelialization and contribution to late thrombosis.

Some current efforts therefore focus on developing DES devoid of polymeric carriers, or otherwise, DES bearing minimal amount of polymeric carriers or at least bearing biodegradable polymers as carriers. These efforts, however, deal with numerous limitations imposed by factors such as the poor adherence of pharmaceutically active agents to bare metal stents and the limited control of drug release (influenced, inter alia, by the drug's dissolution rate).

The control of drug release from drug eluting stents is an important characteristic of the medical device. The rate of drug release is strongly depended on the solid nature, i.e., amorphous vs. crystalline, of the drug, in particular in carrier-free (polymer-free) DES.

Currently employed techniques for coating stents (e.g., dip coating and spray coating) with a drug tend to generate an amorphous layer of the drug. See, for example, Wessely et al. [Arteriosclerosis, Thrombosis, and Vascular Biology 2005, 25:748] which teach a polymer-free stent coated with rapamycin by spray-coating the surface with a rapamycin solution, as well as a device for coating the stent before use. Such an amorphous layer is poorly adhered to the surface. Moreover, these amorphous coatings, when applied on a carrier-free (polymer-free) platform, elute the drug rapidly in a non-controlled manner.

This non-controllable release is often a result of the coatings' high surface area, its high porosity ratio, and its unordered structure. In some cases, the amorphous coating is converted in time (e.g., during storage) to a crystalline coating in a non-controllable manner, such that a non-determined crystalline portion of the drug is formed and/or a crystalline form of the drug is formed at non-determined portions of the stent's surface. Such non-controllable conversion of the amorphous form into a crystalline form further enhances the non-controllability of the drug release and of the coating's stability [See, for example, Belu et al., J. Control. Release, 126 (2) (2008) 111-121].

Accordingly, amorphous drug dissolution rates cannot address pharmacokinetics requirements for restenosis and/or other relevant therapy. Moreover, the amorphous phase nature of many drugs, including rapamycin and paclitaxel, are chemically unstable, resulting in rapid degradation of the drug both under physiological conditions and under storage conditions, thus limiting their commercial and therapeutic value. Hence, DESs manufactured by Translumina, for example, are prepared immediately prior to use [see, for example, Wessely et al., 2005 supra and WO 2004/091684].

In contrast, drugs kept in their crystalline phase are highly stable against such degradation. Thus, efforts are being made to prepare DES loaded with a pharmaceutically active agent in its crystalline form.

Most of the studies conducted with crystalline DES use polymeric carriers to facilitate adherence of crystalline drugs to surfaces.

WO 00/032238 teaches a stent having applied thereof a crystalline drug within or over a polymer coating which coats the stent.

WO 06/063021 teaches a coating composition comprising a polymer and an active agent, wherein the active agent crystallizes following application of the coating composition.

U.S. patent application having Publication No. 20070154554 teaches a crystalline therapeutic agent encapsulated in a biocompatible polymer coating.

U.S. Pat. No. 7,282,213 teaches a method of applying a steroid to a surface of a medical device by depositing a solution of the steroid on the surface to form a crystalline coating, and heating the coating in order to form a coating that is better conformed to the surface.

WO 06/105362 teaches antimicrobial metal-containing coatings.

U.S. patent applications having Publication Nos. 20080097618 and 20060210494 teach crystalline calcium phosphate coatings on medical devices.

WO 08/090,554 teaches electrocoating of a basecoat using a diazonium salt. According to the teachings of this patent application, an improved adherence of therapeutically active agents to the coated surface is obtained.

### SUMMARY OF THE INVENTION

The present inventors have devised and successfully practiced a methodology that enables to provide various surfaces, having applied thereon a layer (continuous or discontinuous) of a crystalline form of a therapeutically active agent, by controlling various parameters of the crystallization process of a drug and/or various parameters of the surface to be coated with a crystalline drug.

According to an aspect of some embodiments of the invention there is provided an article-of-manufacturing comprising an object having a surface and a therapeutically active agent being deposited onto at least a portion of the surface, at least a portion of the therapeutically active agent being in a crystalline form thereof.

According to some embodiments of the invention, the article-of-manufacturing is devoid of a polymeric carrier for carrying the therapeutically active agent.

According to some embodiments of the invention, the crystalline form of the therapeutically active agent is deposited directly onto the surface.

According to some embodiments of the invention, the surface is selected capable of inducing crystallization of at least the portion of the therapeutically active agent.

According to some embodiments of the invention, the article-of-manufacturing further comprising a base layer applied onto the surface, wherein the therapeutically active agent is being deposited onto the base layer.

Hence, according to another aspect of embodiments of the invention there is provided an article-of-manufacturing comprising an object having a surface, a base layer applied onto at least a portion of the surface, and a therapeutically active agent being deposited onto at least a portion of the base layer, at least a portion of the therapeutically active agent being in a crystalline form thereof.

According to some embodiments of the invention, the base layer is designed capable of inducing, promoting, facilitating and/or enhancing a formation of the crystalline form of the therapeutically active age According to some embodiments of the invention, the base layer is designed capable of controlling the kinetic parameters of a release of the therapeutically active agent from the object.

According to some embodiments of the invention, the base layer serves as an additional therapeutically active agent.

According to some embodiments of the invention, the base layer is a non-polymeric layer.

According to some embodiments of the invention, the base layer is a hydrophobic layer and/or a metal oxide layer.

According to some embodiments of the invention, the surface is a conductive or semi-conductive surface and the base layer comprises at least one aryl moiety being electrochemically attached to the surface.

According to some embodiments of the invention, the at least one aryl moiety is selected such that the base layer remains intact upon being subjected to physiological and/or mechanical conditions associated with the object for at least 30 days.

According to some embodiments of the invention, the aryl moiety is formed by electrochemically attaching an aryl diazonium salt to the surface.

According to some embodiments of the invention, the aryl diazonium salt is selected from the group consisting of a 4-(2-hydroxyethyl)-phenyl diazonium salt and a 4-(dodecyloxy)-phenyl diazonium salt.

According to some embodiments of the invention, the base layer is selected capable of interacting with the therapeutically active agent via a hydrophobic interaction, a hydrophilic interaction, a π-interaction and/or any combination thereof.

According to some embodiments of the invention, at least 50% of the therapeutically active agent is in the crystalline form thereof.

According to some embodiments of the invention, at least 90% of the therapeutically active agent is in the crystalline form thereof.

According to some embodiments of the invention, at least 99% of the therapeutically active agent is in the crystalline form thereof.

According to some embodiments of the invention, the article-of-manufacturing further comprising a coat layer coating at least the portion of the surface having deposited thereon the therapeutically active agent.

According to some embodiments of the invention, the coat layer is made from a water-soluble material.

According to some embodiments of the invention, at least 20% of the coat layer dissolves within 1 hour under physiological conditions.

According to some embodiments of the invention, the coat layer comprises a polymeric material.

According to some embodiments of the invention, the water-soluble material is selected from the group consisting of a fatty acid, a lipid, a polyethylene glycol, poly(ethylene-vinyl acetate), poly(butyl methacrylate), poly(styrene-isobutylene-styrene), poly-L-lactide, poly-c-caprolactone, polysaccharide, carboxymethyl cellulose (CMC), dextran, glycerol, chitosan, gelatin, serum albumin, polyvinylpyrrolidone (PVP), arabinogalactan, EUDRAGIT®, an elastic polymer, a surfactant, a gel, a hydrogel and any mixture thereof.

According to some embodiments of the invention, the therapeutically active agent is selected from the group consisting of an anti-restenosis agent, an anti-thrombogenic agent, an anti-platelet agent, an anti-coagulant, a statin, a toxin, an antimicrobial agent, an analgesic, an anti-metabolic agent, a vasoactive agent, a vasodilator, a prostaglandin, a thrombin inhibitor, a vitamin, a cardiovascular agent, an antibiotic, a chemotherapeutic agent, an antioxidant, a phospholipid, an anti-proliferative agent, paclitaxel, rapamycin, and any combination thereof.

According to some embodiments of the invention, the therapeutically active agent is rapamycin.

According to some embodiments of the invention, an amount of the therapeutically active agent that is released upon subjecting the object to physiological conditions for 24 hours is less than 20 percents by weight.

According to some embodiments of the invention, an amount of the therapeutically active agent that is released upon subjecting the object to physiological conditions for 5 days is less than 50 percents by weight.

According to some embodiments of the invention, an amount of the therapeutically active agent that is released upon subjecting the object to physiological conditions for 16 days is less than 70 percents by weight.

According to some embodiments of the invention, the crystalline form of the therapeutically active agent comprises crystals having an average diameter in a range of from 2 to 200 microns.

According to some embodiments of the invention, the crystals have an average diameter in a range of from 75 to 200 microns, and an amount of the therapeutically active agent that is released upon subjecting the object to physiological conditions for 5 days is less than 30 percents by weight.

According to some embodiments of the invention, an amount of the therapeutically active agent that is released upon subjecting the object to physiological conditions for 16 days is less than 60 percents by weight.

According to some embodiments of the invention, the crystals have an average diameter in a range of from 2 to 75 microns.

According to some embodiments of the invention, the therapeutically active agent forms a continuous layer deposited on the surface.

According to some embodiments of the invention, the therapeutically active agent forms a discontinuous layer deposited on the surface.

According to some embodiments of the invention, the therapeutically active agent is deposited onto an outer portion of the surface.

According to some embodiments of the invention, the therapeutically active agent is absent from an inner portion of the surface.

According to some embodiments of the invention, the object is a medical device.

According to some embodiments of the invention, the object is an implantable medical device.

According to some embodiments of the invention, the implantable device is a stent.

According to some embodiments of the invention, the object has a shape selected from the group consisting of a rod, a tubular body, a plate, and a screw.

According to some embodiments of the invention, the article-of-manufacturing further comprising a packaging material, packaging the object and being identified, in or on the packaging material, for use in the treatment of a medical condition treatable by the medical device.

According to an aspect of embodiments of the invention there is provided an article-of-manufacturing comprising a stent having deposited, at least on a portion of a surface thereof, rapamycin, at least 90% of the rapamycin being in a crystalline form thereof.

According to some embodiments of the invention, the stent further comprises a base layer applied on at least a portion of a surface thereof, the base layer being formed by electrochemically attaching an aryl diazonium salt to the surface, and the therapeutically active agent being deposited onto the base layer.

According to some embodiments of the invention, the aryl diazonium salt is selected from the group consisting of a 4-(2-hydroxyethyl)-phenyl diazonium salt and a 4-(dodecyloxy)-phenyl diazonium salt.

According to another aspect of embodiments of the invention there is provided a process of preparing the article-of-manufacturing as described herein, the process comprising:

contacting a surface of the object with a solution containing the therapeutically active agent; and

cooling the surface to a temperature below a temperature of the solution, so as to form the crystalline form of the therapeutically active agent deposited on at least the portion of the surface.

According to some embodiments of the invention, the solution is saturated or supersaturated with the therapeutically active agent.

According to some embodiments of the invention, the solution contains an anti-solvent of the therapeutically active agent.

According to some embodiments of the invention, the anti-solvent is added to the solution subsequent to the contacting of the surface with the solution.

According to some embodiments of the invention, the anti-solvent is added to the solution prior to the contacting of the surface with the solution.

According to some embodiments of the invention, the process further comprising seeding the surface with crystals of the therapeutically active agent prior to the contacting of the surface with the solution.

According to some embodiments of the invention, the solution and the temperature are selected such that at least 50% of the therapeutically active agent is deposited on the surface in the crystalline form.

According to some embodiments of the invention, when wherein the solution and the temperature are selected such that at least a portion of the therapeutically active agent is deposited on the surface in a non-crystalline form, the process further comprises subsequently raising a temperature of the surface contacted with the solution, to thereby convert at least a portion of the non-crystalline form to the crystalline form.

According to some embodiments of the invention, the surface is selected capable of, or is pre-treated so as to be capable of, inducing, promoting, facilitating and/or enhancing crystallization of the therapeutically active agent.

According to some embodiments of the invention, at least 90% of the therapeutically active agent on the surface is in the crystalline form.

According to some embodiments of the invention, the time and/or temperature of a crystallization process are selected so as to enhance an adherence of the crystalline form of the therapeutically active agent to the surface.

According to some embodiments of the invention, the therapeutically active agent forms a continuous layer.

According to some embodiments of the invention, the therapeutically active agent forms a discontinuous layer.

According to some embodiments of the invention, the process further comprising masking a portion of the surface, to thereby obtain a masked portion of the surface, such that the therapeutically active agent is absent from a portion of the surface.

According to some embodiments of the invention, the process further comprising applying a top coat onto the surface having the therapeutically active agent applied thereon.

According to some embodiments of the invention, when the object further comprises a base layer applied onto at least a portion of the surface, the process further comprises, prior to contacting the surface with the solution of the therapeutically active agent, applying the base layer onto the surface.

According to some embodiments of the invention, the surface is a conductive or semi-conductive surface, the layer comprises an aryl moiety and the applying comprises electrochemically attaching at least one aryl moiety substituted by at least one diazonium moiety to the surface.

Accordingly, according to a further aspect of embodiments of the invention there is provided a process of preparing an object having a conductive or semi-conductive surface, at least one aryl moiety being electrochemically attached to the surface and forming a base layer of the at least one aryl moiety, and a therapeutically active agent being applied onto the base layer, at least a portion of the therapeutically active agent being in a crystalline form thereof, the process comprising:

electrochemically attaching at least one aryl moiety substituted by at least one diazonium moiety to the conductive surface, to thereby obtain the object having the base layer of the at least one aryl moiety being electrochemically attached to the surface;

contacting a surface of the object having the base layer electrochemically attached to the surface with a solution containing the therapeutically active agent; and

cooling the surface to a temperature below a temperature of the solution, so as to form the crystalline form of the therapeutically active agent deposited on at least the portion of the surface.

According to some embodiments of the invention, the aryl moiety is selected capable of inducing, promoting, facilitating and/or enhancing crystallization of the therapeutically active agent.

According to some embodiments of the invention, the at least one aryl moiety substituted by at least one diazonium moiety is selected from the group consisting of a 4-(2-hydroxyethyl)-phenyl diazonium salt and a 4-(dodecyloxy)-phenyl diazonium salt.

According to some embodiments of the invention, the object is a medical device.

According to some embodiments of the invention, the object is a stent.

According to an additional aspect of embodiments of the invention there is provided a method of treating a subject having a medical condition in which implanting a medical device is beneficial, the method comprising:

implanting the medical device as described herein within the subject, thereby treating the medical condition.

According to some embodiments of the invention, the medical condition is selected from the group consisting of a cardiovascular disease, atherosclerosis, thrombosis, stenosis, restenosis, a cardiologic disease, a peripheral vascular disease, an orthopedic condition, a proliferative disease, an infectious disease, a transplantation-related disease, a degenerative disease, a cerebrovascular disease, a gastrointestinal disease, a hepatic disease, a neurological disease, an autoimmune disease, and an implant-related disease.

According to yet a further aspect of embodiments of the invention there is provided an apparatus for performing the process described herein, the apparatus comprising;

a rod supporting the object;

a cooling mechanism being in thermal communication with the rod, for cooling the rod;

and

a receptacle for holding a solution comprising the therapeutically active agent,

such that when the object is supported by the rod and the receptacle holds the solution comprising the therapeutically active agent, at least a portion of the surface of the object is in fluid communication with the solution comprising the therapeutically active agent.

According to still a further aspect of embodiments of the invention there is provided an apparatus for preparing an object having a surface and a crystalline form of a therapeutically active agent being applied onto the surface, the apparatus comprising;

a rod supporting the object;

a cooling mechanism being in thermal communication with the rod, for cooling the rod; and

a receptacle for holding a solution comprising the therapeutically active agent,

such that when the object is supported by the rod and the receptacle holds the solution comprising the therapeutically active agent, at least a portion of the surface of the object is in fluid communication with the solution comprising the therapeutically active agent.

According to some embodiments of the invention, the rod is a hollow rod and the cooling mechanism comprises a coolant for flowing through the hollow rod.

According to some embodiments of the invention, the cooling mechanism further comprises a device for cooling the coolant; and a device for causing the coolant to flow through the rod.

According to some embodiments of the invention, the cooling mechanism comprises a cooled reservoir, being in direct communication with the rod.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a graph plotting the percentage of 0.1 mg (blank), 3 mg (black) and 15 mg (gray) rapamycin which remains dissolved in a solution of 1 ml ethyl acetate and 20 ml n-hexane at 0° C. (squares), 15° C. (triangles) and 30° C. (circles), as a function of time;

FIG. 2 is a schematic illustration of a system for inducing drug deposition on the surface of a stent, according to embodiments of the invention;

FIGS. 3A-C present SEM (scanning electron microscopy) images of rapamycin deposition on DS-06-electrocoated CrCo stents, effected by cooling the stents and immersing the stents in a solution of 15 mg rapamycin (FIG. 3A) or 17.5 mg rapamycin (FIGS. 3B and 3C) in 1 ml ethyl acetate+20 ml n-hexane for 120 minutes (FIGS. 3A and 3B) or 100 minutes (FIG. 3C), as described in FIG. 2 and in Example 2, using a high coolant flow rate;

FIG. 4 presents photographs at magnifications of ×2 (left panel), ×4 (middle panel) and ×8 (right panel) of rapamycin deposition on DS-06-electrocoated CrCo stents performed by cooling the stents and immersing the stents for 100 minutes in a solution of 15 mg rapamycin in 1 ml ethyl acetate+20 ml n-hexane at 0° C., as described in FIG. 2 and in Example 2, using a high coolant flow rate;

FIG. 5 presents a schematic illustration of a part of a drug deposition system according to embodiments of the invention, where a partially expanded stent with a conical configuration was placed on a hollow rod through which a coolant flows, such that only the narrow region of the stent is in contact with the rod;

FIG. 6 presents photographs at magnifications of ×2 (lower left panel), ×4 (upper left and middle, and lower right and middle panels) and ×8 (upper right panel) of rapamycin deposition on DS-06-electrocoated CrCo stents, performed by cooling the stents and immersing the stents for 100 minutes in a solution of 15 mg rapamycin in 1 ml ethyl acetate+20 ml n-hexane, as described in FIGS. 2 and 5 and in Example 2, using a high coolant flow rate;

FIGS. 7A-C present photographs (FIG. 7A) at magnifications of ×4 (left panel) and ×8 (right panel) and SEM images (FIGS. 7B and 7C) of rapamycin deposition on DS-06-electrocoated CrCo stents, performed by cooling the stents and immersing the stents for 120 minutes (FIGS. 7A and 7B) or 60 minutes (FIG. 7C) in a solution of 25 mg rapamycin in 1 ml ethyl acetate+20 ml n-hexane, as described in FIG. 2 and in Example 2, using a high coolant flow rate;

FIGS. 8A-B present SEM images at magnifications of ×1000 (upper panel) and ×300 (lower panel) of rapamycin deposition on DS-06-electrocoated CrCo stents, performed by cooling the stents, and immersing the stents for 30 minutes (FIG. 8A) or 60 minutes (FIG. 8B) in a solution of 25 mg rapamycin in 1 ml ethyl acetate+20 ml n-hexane, as described in FIG. 2 and in Example 2, using reduced cooling of the stent followed by incubation of the stents in the solution overnight at room temperature, and clearly showing deposition of crystalline rapamycin after 60 minutes immersion (FIG. 8B);

FIG. 9 presents a plot showing an X-ray diffraction spectrum of rapamycin deposited on a DS-06-electrocoated stent surface from a solution of 15 mg in 1 ml ethyl acetate and 20 ml hexane, as described in FIG. 2 and Example 2, using a high coolant flow rate (10 ml/minute) for 30 minutes; the spectrum shows that the rapamycin is amorphous;

FIG. 10 presents a graph showing an X-ray diffraction spectrum of rapamycin spray-coated onto a DS-06-electrocoated stent surface using a solution of 1% rapamycin (weight/volume) in ethyl acetate; the spectrums show that the rapamycin is amorphous;

FIG. 11 presents an X-ray diffraction spectrum of rapamycin deposited on a DS-06-electrocoated stent surface from a solution of 15 mg in 1 ml ethyl acetate and 20 ml hexane, as described in FIG. 2 and Example 2 using a high coolant flow rate (10 ml/minute) for 30 minutes, followed by 120 minutes at room temperature; the spectrum shows that the rapamycin is crystalline (red lines indicate spectral lines of isomorph II rapamycin crystals as reported in the literature);

FIG. 12 presents an X-ray diffraction spectrum of rapamycin deposited on a DS-06-electrocoated stent surface from a solution of 15 mg in 1 ml ethyl acetate and 20 ml hexane, as described in FIG. 2 and Example 2 using a moderate coolant flow rate (5 ml/minute) for 60 minutes; the spectrum shows that the rapamycin is crystalline (red line indicates spectrum of isomorph II rapamycin crystals as reported in the literature);

FIGS. 13A-B present a photograph (FIG. 13A) and SEM images (FIG. 13B) of rapamycin crystal deposition on DS-06-electrocoated CrCo stents, obtained by immersing the stents for 64 hours in a solution of 1 ml ethyl acetate with 25 mg rapamycin, to which 25 ml n-hexane was added at a rate of 0.5 ml/minute;

FIG. 14 presents SEM images at magnifications of ×300 (left panel), ×600 (middle panel) and ×200 (right panel) showing rapamycin crystal deposition on DS-06-electrocoated CrCo stents, obtained by immersing the stents for 48 hours in a solution of 1 ml ethyl acetate with 25 mg rapamycin, to which 25 ml n-hexane was added at a rate of 0.5 ml/minute;

FIG. 15 presents SEM images showing rapamycin deposition on DS-06-electrocoated CrCo stents, obtained by immersing the stents for 50 minutes in a solution of 4 ml ethyl acetate with 100 mg rapamycin, to which 22 ml n-hexane was added at a rate of 0.5 ml/minute;

FIGS. 16A-B presents SEM images at magnifications of ×30,000 (FIG. 16A) and ×700 (FIG. 16B) showing rapamycin deposition on DS-06-electrocoated CrCo stents, obtained by immersing the stents for 110 minutes in a solution of 1 ml ethyl acetate with 100 mg rapamycin, to which 22 ml n-hexane was added at a rate of 0.2 ml/minute;

FIGS. 17A-C presents SEM images at various magnifications, showing rapamycin deposition on DS-06-electrocoated CrCo stents, obtained by immersing the stents for 100 minutes in a solution of 1 ml ethyl acetate with 10 mg rapamycin, to which 20 ml n-hexane was added at a rate of 0.2 ml/minute;

FIG. 18 presents a schematic illustration of a system for inducing deposition on a stent by placing the stent on a solid rod cooled by a cold reservoir, according to embodiments of the invention;

FIGS. 19A-B present photographs (FIG. 19A) and SEM images (FIG. 19B) at various magnifications, showing rapamycin deposition on DS-04-electrocoated CrCo stents, obtained by cooling the stents and immersing the stents in a solution of 4 ml ethyl acetate with 100 mg rapamycin, to which 16 ml n-hexane was added at a rate of 0.5 ml/minute, as described in FIG. 18;

FIG. 20 presents photographs (upper images) at magnifications of ×4 (upper left) and ×8 (upper right) and SEM images (lower images) at magnifications of ×250 (lower left) and x 1000 (lower right), showing the surface of bare YUKON® stainless steel stents;

FIGS. 21A-B present photographs showing amorphous rapamycin deposition on DS-06-electrocoated YUKON® stainless steel stents, obtained by cooling the stents and immersing the stents for 30 minutes in a solution of 15 mg rapamycin in 1 ml ethyl acetate+20 ml n-hexane, as described in FIG. 2 and Example 2, using a high coolant flow rate (FIG. 21A) and crystalline rapamycin deposition on these stainless steel stents, obtained by further immersing the stents in the solution for 2 hours at room temperature (FIG. 21B);

FIGS. 22A-B present photographs (FIG. 22A) and SEM images (FIG. 22B), at various magnifications, showing crystalline rapamycin deposition on the surface of a DS-06-electrocoated CrCo stent, obtained by cooling the stents and immersing the stents for 30 minutes in a solution of 15 mg rapamycin in 1 ml ethyl acetate+20 ml n-hexane, as described in FIG. 2 and Example 2, using a high coolant flow rate, and for an additional 2 hours at room temperature;

FIG. 23 presents an SEM image of a piece of crystalline rapamycin broken off of the surface of a DS-06-electrocoated YUKON® stainless steel stent (red arrows point to visible crystals);

FIGS. 24A-B present photographs at magnifications of ×400, showing rapamycin deposition on DS-06-electrocoated YUKON® stainless steel stents, obtained by cooling the stents and immersing the stents for 30 minutes with cooling as described in FIG. 2 and Example 2, using a high coolant flow rate, and for a further 2 hours at room temperature, in a solution containing 15 mg rapamycin dissolved in 1 ml ethyl acetate (FIG. 24A) and 2 ml ethyl acetate (FIG. 24B)+20 ml n-hexane;

FIGS. 25A-25C present photographs (FIGS. 25A and 25B) and a SEM image (FIG. 25C), showing rapamycin deposition on the surface of a DS-06-electrocoated YUKON® stainless steel stent following incubation in a solution of 15 mg rapamycin dissolved in 1 ml ethyl acetate+20 ml n-hexane for 2 hours at room temperature without prior cooling of the stent (bare patches on the surface are circled in red);

FIGS. 26A-B presents photographs at various magnifications, showing rapamycin deposition on the surface of a DS-06-electrocoated YUKON® stainless steel stent following incubation in a solution of 25 mg rapamycin dissolved in 1 ml ethyl acetate+20 ml n-hexane for 72 hours at room temperature without prior seeding of the stent;

FIGS. 27A-B present SEM images at various magnifications, showing rapamycin deposition on the surface of a DS-06-electrocoated CrCo stent following incubation in a solution of 25 mg rapamycin dissolved in 1 ml ethyl acetate+20 ml n-hexane for 72 hours at room temperature without prior seeding of the stent;

FIGS. 28A-C present photographs, at various magnifications, showing rapamycin deposition on DS-06-electrocoated stainless steel rods obtained by immersing the rods for 30 minutes with cooling of the rods as described in FIG. 2 and Example 2, using a high coolant flow rate, and for a further 30 minutes (FIG. 28A), 1 hour (FIG. 28B) and 2 hours (FIG. 28C) at room temperature in a solution of 15 mg rapamycin in 1 ml ethyl acetate+20 ml n-hexane;

FIG. 29 presents a graph plotting the weight of deposited rapamycin on a DS-06-electrocoated stainless steel rod over the course of 2 hours of incubation at room temperature following 30 minutes of cooling of the rod, showing the amorphous rapamycin (point A) disappearing and being replaced by crystalline rapamycin (points B, C and D);

FIG. 30 is a graph generally plotting the dependence of nucleation rate and crystal growth rate on crystallization driving force;

FIGS. 31A-D present photographs showing rapamycin deposition on the surface of a DS-06-electrocoated YUKON® stainless steel stent following 2 hours (FIG. 31A), 1 hour (FIG. 31B), 30 minutes (FIG. 31C) and 15 minutes (FIG. 31D) incubation in a solution of 10 mg rapamycin in 1 ml ethyl acetate+20 ml n-hexane, with cooling of the stent as described in FIG. 2 and Example 2, using a moderate coolant flow rate;

FIG. 32 presents comparative plots showing the weight of deposited rapamycin on the surface of a DS-06-electrocoated stent obtained as described in FIG. 2, when using a moderate coolant flow rate (open squares), and during incubation at room temperature after using a high coolant flow rate (filled squares);

FIGS. 33A-C present photographs, at various magnifications, showing the surface of a DS-06-electrocoated stainless steel stent (Johnson & Johnson) (FIG. 33A), the stent surface following seeding by sonicating the stent with a homogeneous crystalline rapamycin powder in n-hexane (FIG. 33B), and the stent surface following deposition of rapamycin onto the seeded surface (FIG. 33C);

FIG. 34 presents a SEM image showing the homogeneity of rapamycin crystals on the surface of a stainless steel stent (Johnson & Johnson) following seeding by sonicating the stent with a crystalline rapamycin powder in n-hexane;

FIGS. 35A-B presents photographs at a magnification of ×4 (FIG. 35A) or without magnification (FIG. 35B) of rapamycin crystallization on DS-06-electrocoated CrCo stents, obtained by incubating the stent for 30 minutes with cooling and then overnight at room temperature in a solution of 25 mg rapamycin in 1 ml ethyl acetate+20 ml n-hexane;

FIG. 36 presents comparative plots showing the release of crystalline rapamycin (blank squares) and amorphous (filled diamonds; control) rapamycin from the surface of rapamycin-coated DS-06-electrocoated CrCo stents, prepared as described in Example 10;

FIG. 37 presents comparative plots showing the total rapamycin release from the surface of DS-06-electrocoated CrCo rods coated with amorphous rapamycin by deposition from a solution of 15 mg rapamycin in 1 ml ethyl acetate and 20 ml n-hexane using a coolant flow rate of 10 ml/minute (open squares), and by spray-coating with 1% rapamycin solution in ethyl acetate (filled squares), as a function of time of incubation under physiological conditions;

FIGS. 38A-B present comparative plots showing the total rapamycin release from the surface of stainless steel rods (FIG. 38A) and stents (FIG. 38B) coated with crystalline rapamycin (squares) or amorphous rapamycin (diamonds; control), prepared as described in Example 12, as a function of time of incubation under physiological conditions;

FIG. 39 presents photographs showing crystalline rapamycin remaining on the surface of a YUKON® stainless steel stent following incubation under physiological conditions for 0 hours (upper left panel), 8 hours (upper middle panel), 3 days (upper right panel), 7 days (lower left panel) and 17 days (lower right panel);

FIG. 40 presents comparative plots showing the effect of crystal size (150-200 microns (blank circles) and 25-40 microns (filled squares)) on the release of crystalline rapamycin from the surface of a YUKON® stainless steel stent;

FIGS. 41A-B present comparative plots showing the total rapamycin release from the surface of DS-06-electrocoated CrCo stents coated with crystalline rapamycin by deposition from a solution of 3 mg rapamycin in 1 ml ethyl acetate and 20 ml n-hexane using a coolant flow rate of 6 ml/minute (open squares), and from control CrCo stents coated with amorphous rapamycin by spray-coating with 1 rapamycin solution in ethyl acetate (filled squares), as a function of time of incubation under physiological conditions, without (FIG. 41A) and with (FIG. 41B) expansion of the stent prior to incubation;

FIG. 42 presents comparative plots showing the total rapamycin release from the surface of DS-06-electrocoated CrCo stents coated with crystalline rapamycin by deposition from a solution of 3 mg rapamycin in 1 ml ethyl acetate and 20 ml n-hexane using a coolant flow rate of 6 ml/minute (open squares) or from CYPHER® stents (filled squares) as a function of time of incubation under physiological conditions;

FIGS. 43A-D present photographs, at various magnifications, showing crystalline rapamycin deposition on the surface of DS-06-electrocoated CrCo stents, obtained by cooling the stents and immersing the stents for 30 minutes in a solution of 15 mg rapamycin in 1 ml ethyl acetate+20 ml n-hexane, as described in FIG. 2 and Example 2, using a high coolant flow rate, and further immersing the stents in the solution for 2 hours at room temperature, before (FIGS. 43A and 43B) and after (FIGS. 43C and 43D) expansion of the stent;

FIGS. 44A-D present photographs, at various magnifications, showing crystalline rapamycin deposition on the surface of DS-06-electrocoated CrCo stents, obtained by cooling the stents and immersing the stents for 30 minutes in a solution of 15 mg rapamycin in 1 ml ethyl acetate+20 ml n-hexane, as described in FIG. 2 and Example 2, using a high coolant flow rate, and further immersing the stents in the solution for 2 hours at room temperature, and applying a water-soluble sodium carboxymethyl cellulose (CMC) top coat, before (FIGS. 44A and 44B) and after (FIGS. 44C and 44D) expansion of the stent;

FIG. 45 presents a photograph showing crystalline rapamycin deposited on the surface of a CrCo stent, by cooling the stents and immersing the stents for 30 minutes in a solution of 15 mg rapamycin in 1 ml ethyl acetate+20 ml n-hexane, as described in FIG. 2 and Example 2, using a high coolant flow rate, and further immersing the stents in the solution for 2 hours at room temperature, without electrocoating the stent prior to rapamycin deposition, demonstrating a deposition of crystalline rapamycin that is similar to that performed on electrocoated stent;

FIGS. 46A-D present photographs (FIGS. 46B and 46D) and SEM images (FIGS. 46A and 46C), at various magnifications, showing non-continuous rapamycin deposition on the surface of DS-06-electrocoated CrCo stents incubated in a solution containing 2.5 mg rapamycin with moderate cooling for 45 minutes (FIG. 46A) and in a solution containing 7.5 mg rapamycin with moderate cooling for 10 minutes (FIG. 46B), as well as continuous rapamycin deposition on the surface of DS-06-electrocoated CrCo stents incubated in a solution containing 15 mg rapamycin with strong cooling for 30 minutes followed by 2 hours at room temperature (FIGS. 46C and 46D);

FIGS. 47A-D present photographs, at various magnifications, showing a non-continuous layer of crystalline rapamycin on the surface of DS-06-electrocoated CrCo stents incubated in a solution containing 3 mg rapamycin with moderate cooling of the stent for 60 minutes (FIGS. 47A and 47B) and continuous layer of crystalline rapamycin deposition on the surface of DS-06-electrocoated CrCo stents incubated in a solution containing 15 mg rapamycin with strong cooling of the stent for 30 minutes followed by 2 hours at room temperature (FIGS. 47C and 47D) following expansion of the stents;

FIGS. 48A-B present photographs of rapamycin deposition on the surface of a DS-06-electrocoated CrCo stent seeded by dip-coating the stent in the upper phase of a dispersion of ground rapamycin in n-hexane (FIG. 48A) or by sonication of the dispersion with the stent (FIG. 48B);

FIGS. 49A-B present photographs showing an exemplary system for preparing CrCo stent having crystalline rapamycin deposited on the external side but not on the internal side of the stent's surface, while utilizing an expandable polymeric tube, and a seeding solution, prior to deposition of crystallized rapamycin;

FIGS. 50A-D present photographs showing a DS-06-electrocoated CrCo stent surface with rapamycin deposited on the external side but not on the internal side (external side in focus in FIGS. 50A and 50B, internal side in focus in FIGS. 50C and 50D) at a magnification of ×2 (FIGS. 50B and 50D) or ×4 (FIGS. 50A and 50C);

FIGS. 51A-D present SEM images showing a DS-06-electrocoated CrCo stent surface with rapamycin deposited on the external side but not on the internal side;

FIGS. 52A-B present photographs showing rapamycin deposition on the surface of a DS-06-electrocoated stainless steel tube partially coated with carboxymethyl cellulose, before (FIG. 52A) and after (FIG. 52B) washing away the carboxymethyl cellulose;

FIG. 53 presents photographs showing the surface of a DS-06-electrocoated CrCo stent incubated in a solution containing 7.5 mg rapamycin with moderate cooling of the stent for 10 minutes without being seeded beforehand;

FIGS. 54A-B present a photograph (FIG. 54A) and a SEM image (FIG. 54B) showing rapamycin deposition on the surface of a DS-06-electrocoated CrCo stent coated with poly(lactate-co-glycolate) and incubated in a solution containing 3 mg rapamycin with moderate cooling of the stent for 60 minutes; and

FIG. 55 presents comparative plots showing the release profile of a crystalline rapamycin deposited on a non-electrocoated stent (denoted as “bare”; black squares) and on an electrocoated stent (denoted as “electrocoated; blank squares).

### DESCRIPTION OF SPECIFIC EMBODIMENTS OF THE INVENTION

The present invention, in some embodiments thereof, relates to surfaces having applied thereon therapeutically active agents and, more particularly, but not exclusively, to articles-of-manufacturing such as medical devices having applied thereon a therapeutically active agent, at least a portion of the therapeutically active agent being in a crystalline form thereof, and to processes and apparatus utilized for preparing same.

Embodiments of the present invention relate to objects having a surface and a base layer onto which the therapeutically active agent is deposited.

Some embodiments of the present invention relate to objects having the therapeutically active agent deposited directly on a surface thereof.

Further embodiments of the present invention relate to processes of preparing the described articles of manufacturing.

As discussed hereinabove, current methodologies for manufacturing drug-eluting medical devices such as drug-eluting stents (DES) involve either deposition of a polymeric carrier in which the drug is dispersed, or direct deposition of the drug on the surface of the device. As further discussed hereinabove, the use of polymeric materials as drug carriers in drug-eluting devices is associated with adverse side effects, whereby the currently practiced technologies for direct deposition of drugs on the surfaces of medical devices are associated with poor adherence of the drug to the surface, and further, typically result is deposition of an amorphous form of the drug. Both the poor adherence and the amorphous form of the drug result is a non-controllable release of the drug.

The present inventors have now devised and successfully practiced a novel methodology for depositing therapeutically active agents onto a surface, a methodology which is highly beneficial for coating medical devices. This methodology is based on depositing on an object's surface a crystalline form of the therapeutically active agent. This methodology results in a well-adhered deposition of the therapeutically active agent onto the surface, which is further characterized by a desirable and controllable release profile.

As described in detail in the Examples section that follows, the methodology presented herein is preferably effected by cooling of the surface to be coated to a temperature below that of a solution containing the therapeutically active agent which contacts the surface. The methodology optionally further includes seeding the surface with small crystals of the therapeutically active agent, thereby enhancing crystallization. As further demonstrated in the Examples section that follows, various parameters of the practiced methodology can be manipulated, so as to affect the release profile of the therapeutically active agent.

Thus, using the methodology described herein, objects having deposited on a surface thereof a therapeutically active agent which is, at least in part, in a crystalline form thereof, are obtained. Using the methodology described herein circumvents the need to use a polymeric drug carrier in order to achieve the desirable characteristics of drug-eluting medical devices.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

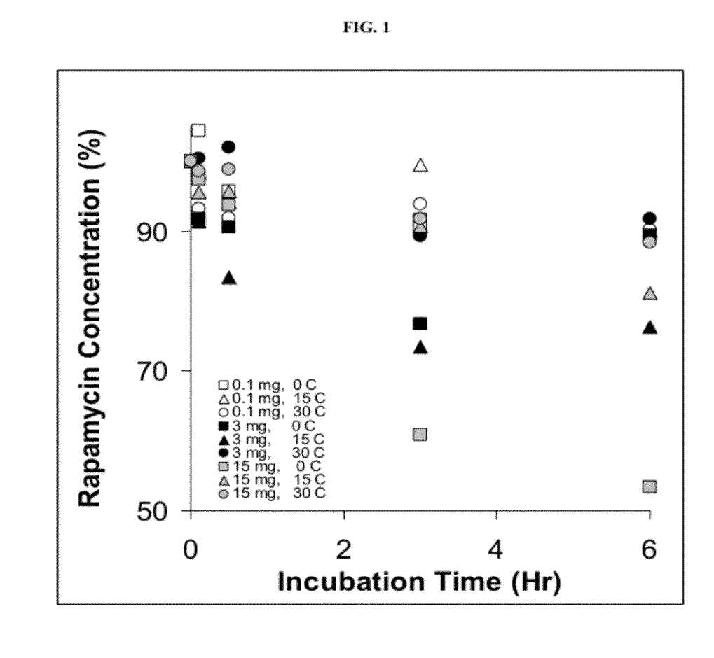

Referring now to the drawings, FIG. 1 presents data indicating that the concentration of an agent in a supersaturated solution and cooling of the solution, effect deposition of a crystalline form of rapamycin, as an exemplary therapeutically active agent.

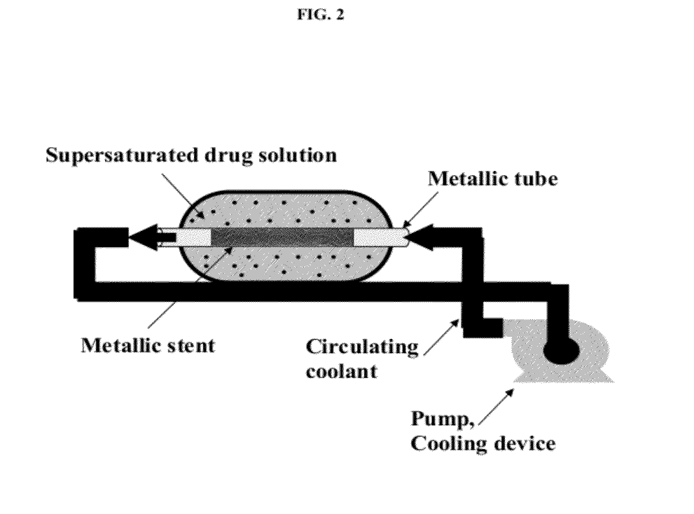

FIG. 2 describes an exemplary system for depositing an agent from a solution onto a surface (e.g., a surface of a stent), according to some embodiments of the invention.

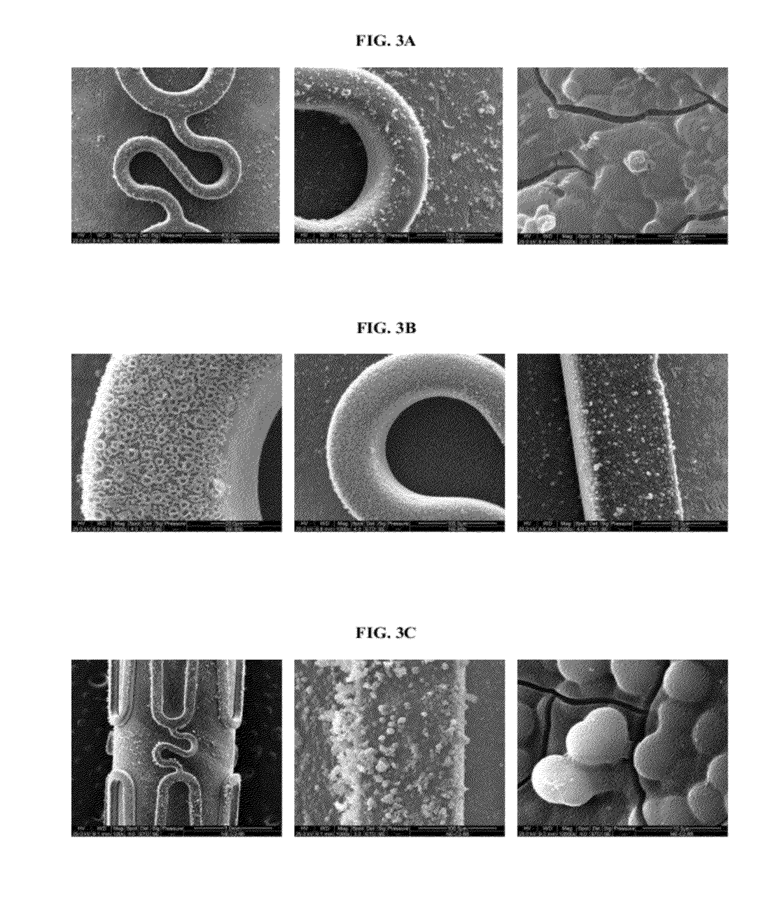

FIGS. 3-8 show deposition of rapamycin on stent surfaces under various conditions.

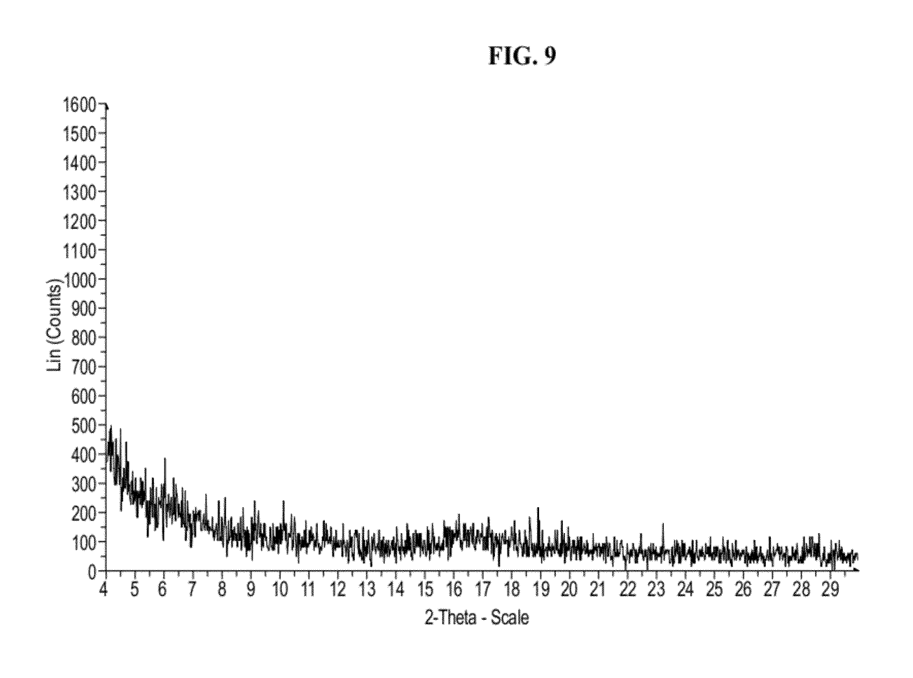

FIGS. 9-12 present data demonstrating amorphous deposition of rapamycin as a result of strong deposition driving forces or spray-coating, and crystalline deposition of rapamycin as a result of moderate deposition driving forces or incubation of amorphous depositions at room temperature.

FIGS. 13-19 present images of crystalline rapamycin obtained on stents,

FIGS. 20-23 present images showing rapamycin crystals growing from the surface of a seeded stent.

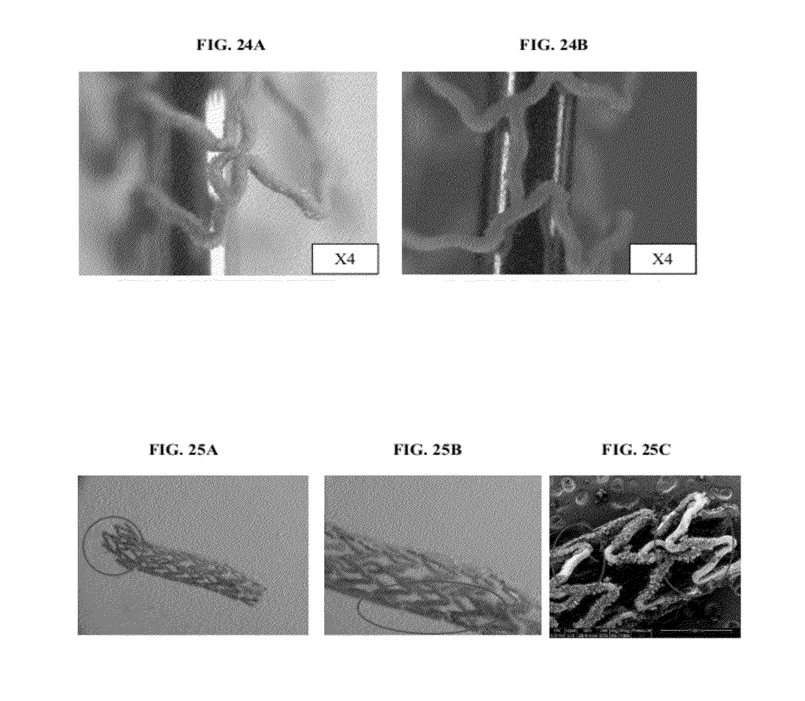

FIG. 24 presents images demonstrating the effect of rapamycin concentration on size of rapamycin crystals.

FIG. 25 presents images demonstrating the effect of cooling on crystal growth.

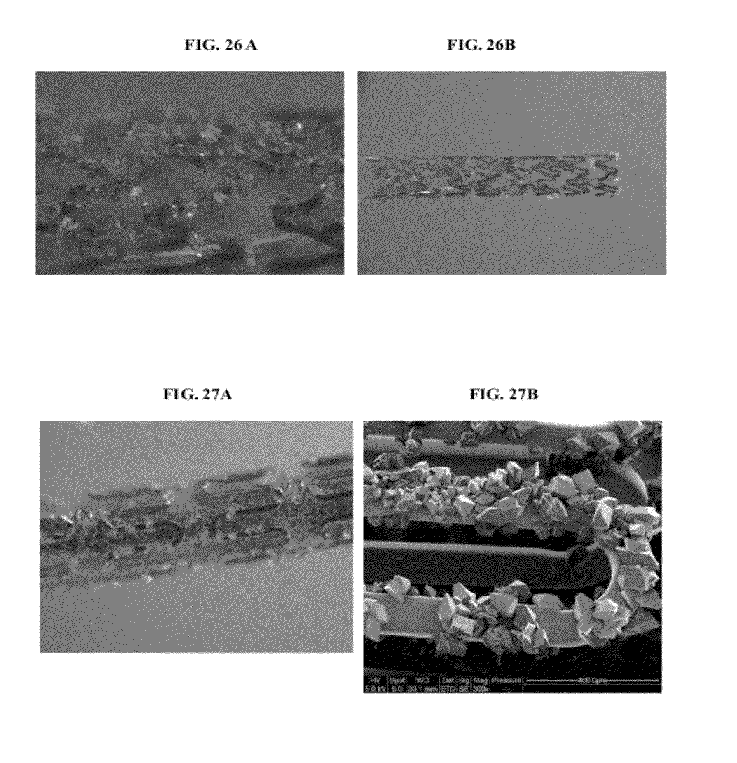

FIGS. 26, 27 and 53 present images demonstrating the enhancing effect of seeding and rapamycin concentration on rapamycin crystal growth.

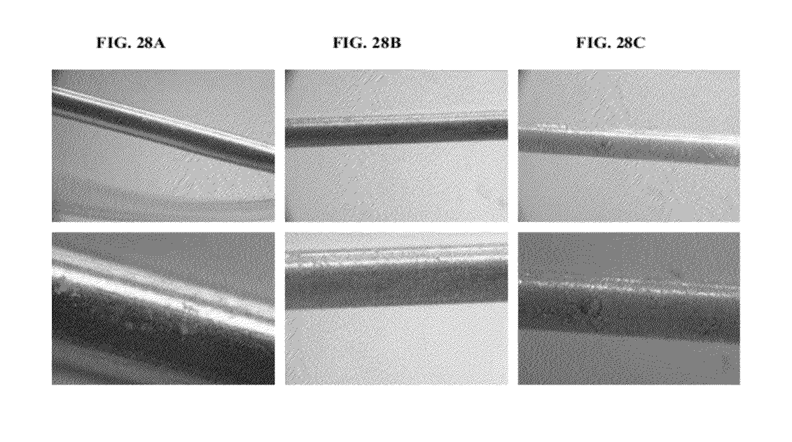

FIGS. 28, 29, 31 and 32 demonstrate the gradual development of crystalline rapamycin during the crystallization process.

FIG. 30 is a diagram describing the effect of deposition driving force on crystal nucleation and growth rates.

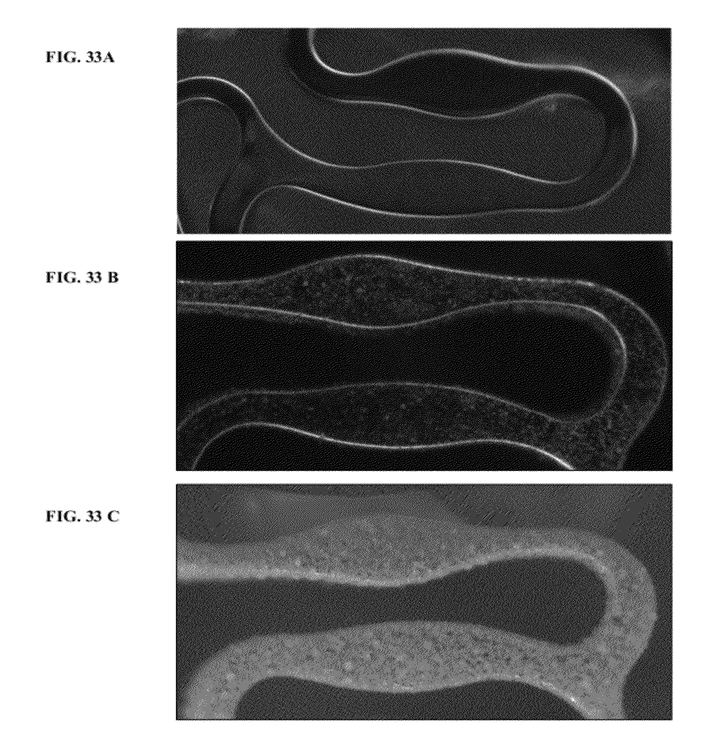

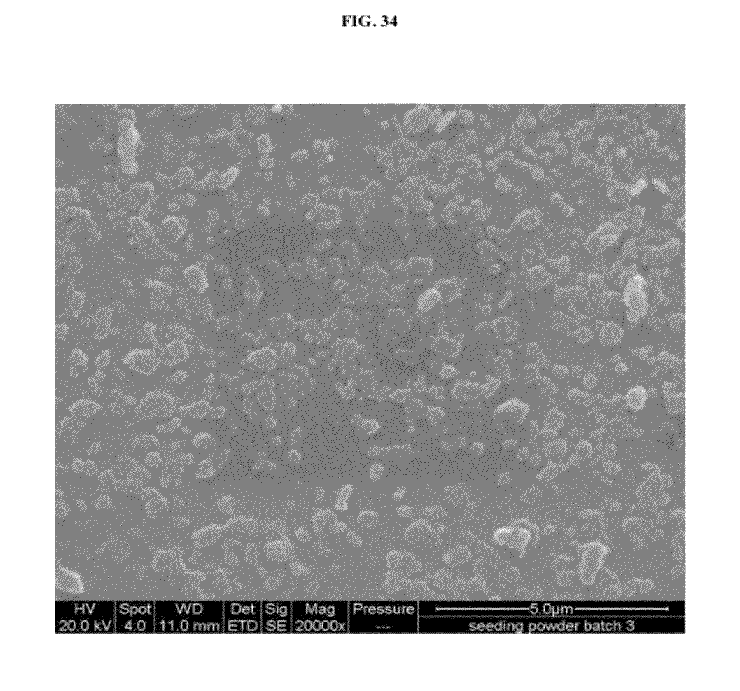

FIGS. 33 and 34 present images showing the seeding of a stent according to an exemplary method, and the crystalline rapamycin deposited on the seeded stent.

FIGS. 36-42 present data demonstrating that crystalline rapamycin is released more slowly than amorphous rapamycin, and that the rate of release depends on crystal size.

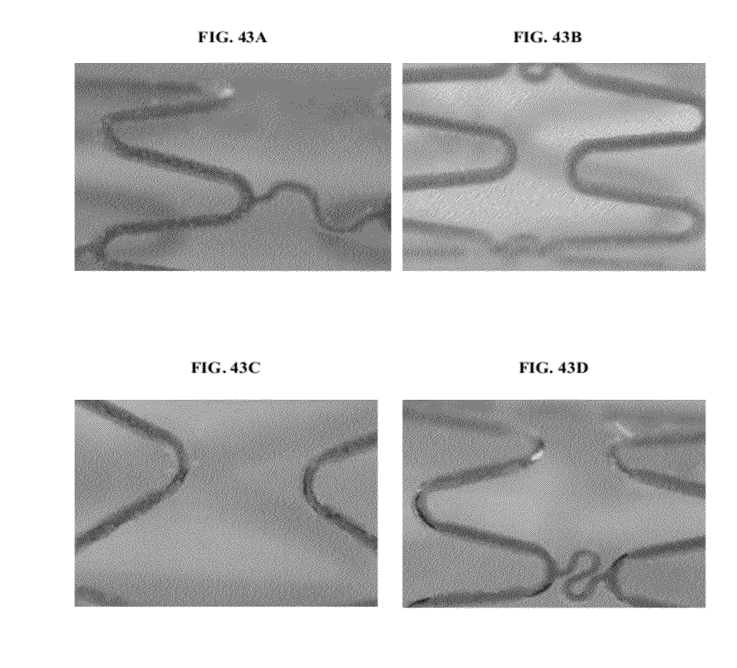

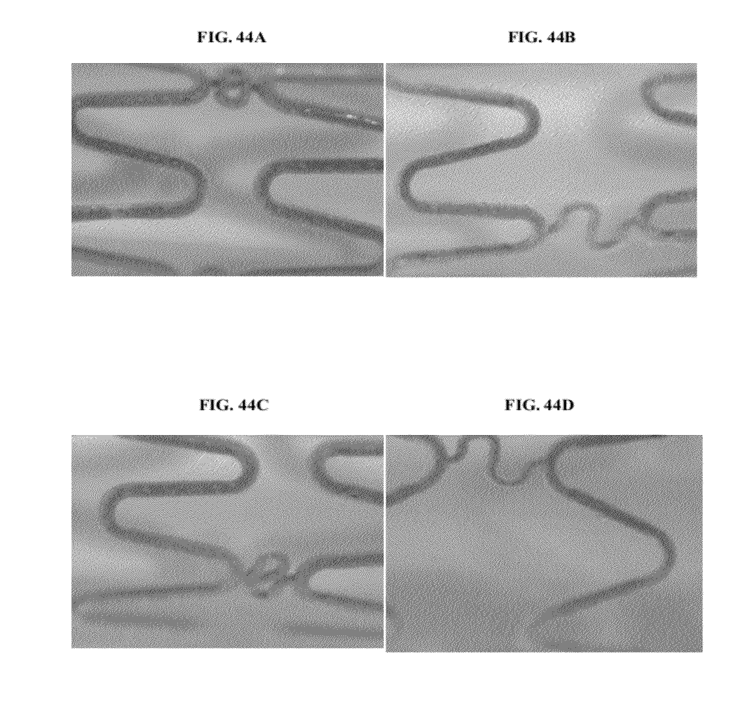

FIGS. 43 and 44 present images showing that coating a layer of crystalline rapamycin with a top-coat can protect the layer from the effects of mechanical forces.

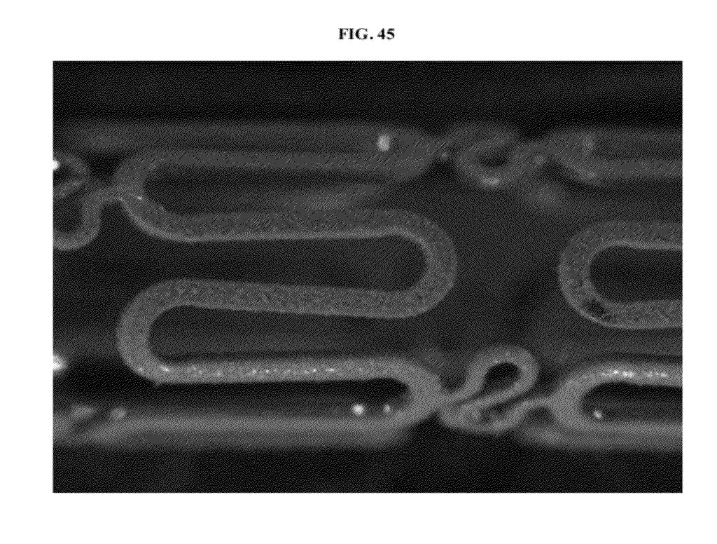

FIGS. 45 and 55 show crystalline rapamycin deposited on a non-electrocoated metal surface and the release profile of rapamycin therefrom, as compared to crystalline rapamycin deposited on electrocoated metal surface.

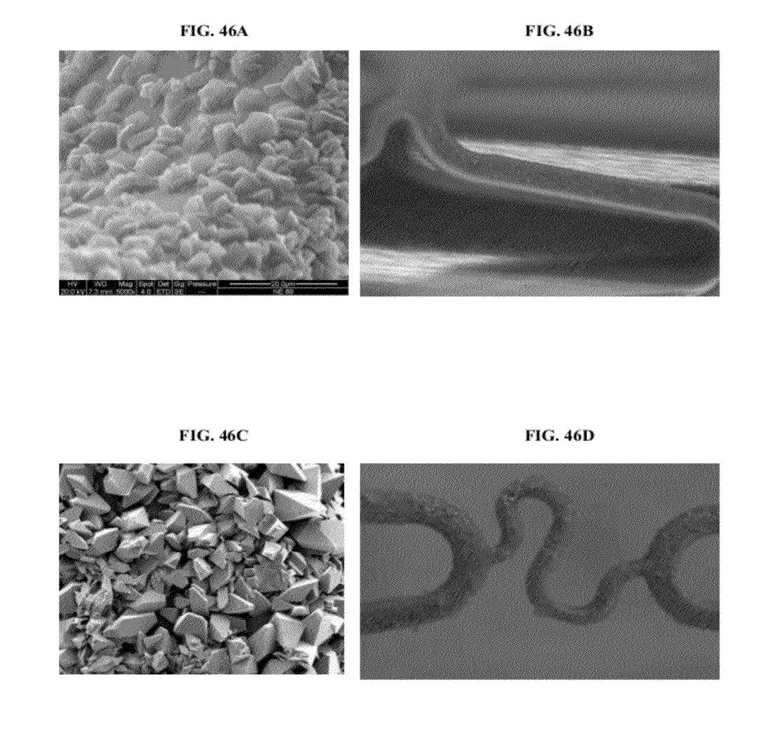

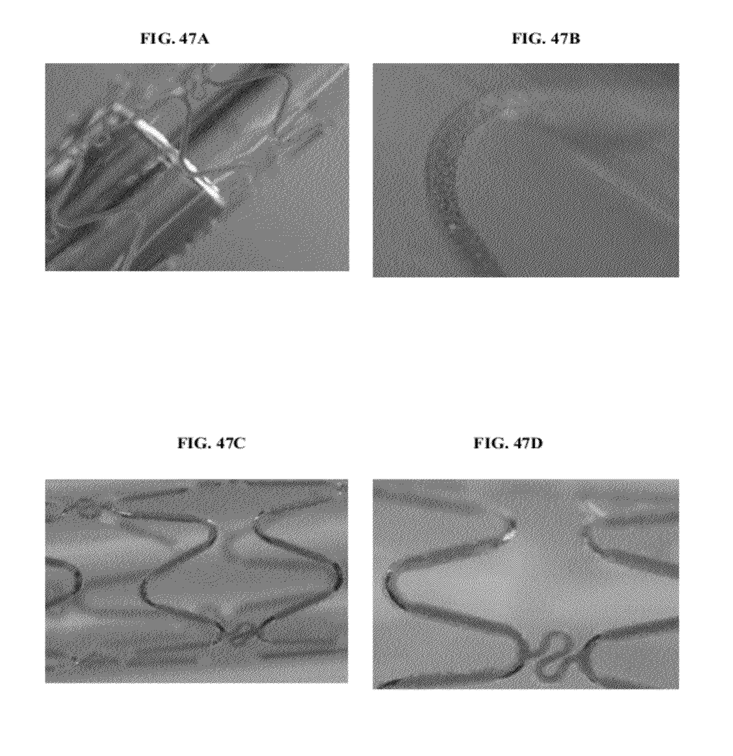

FIGS. 46 and 47 present images showing that a non-continuous layer of crystalline rapamycin is more resilient to the effects of mechanical forces than is a continuous layer.

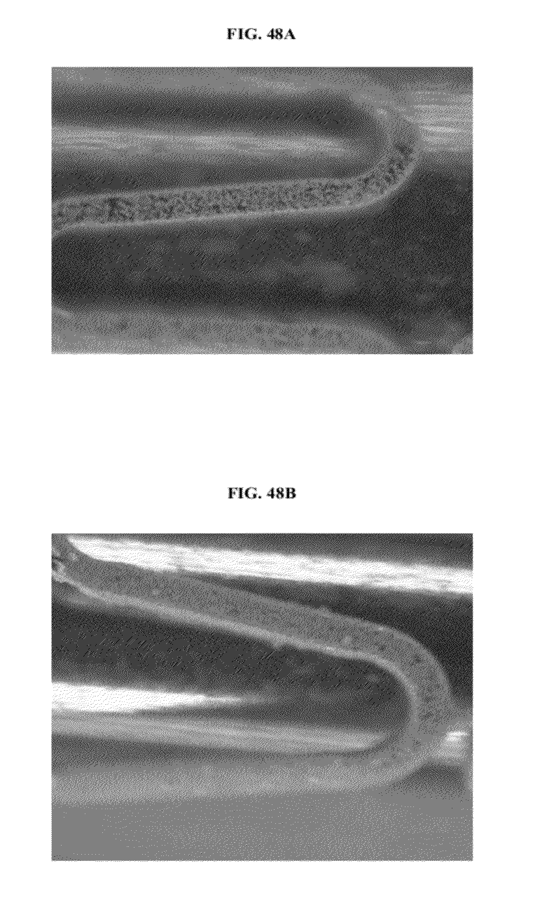

FIG. 48 presents images showing that crystal density is affected by the seeding methodology.

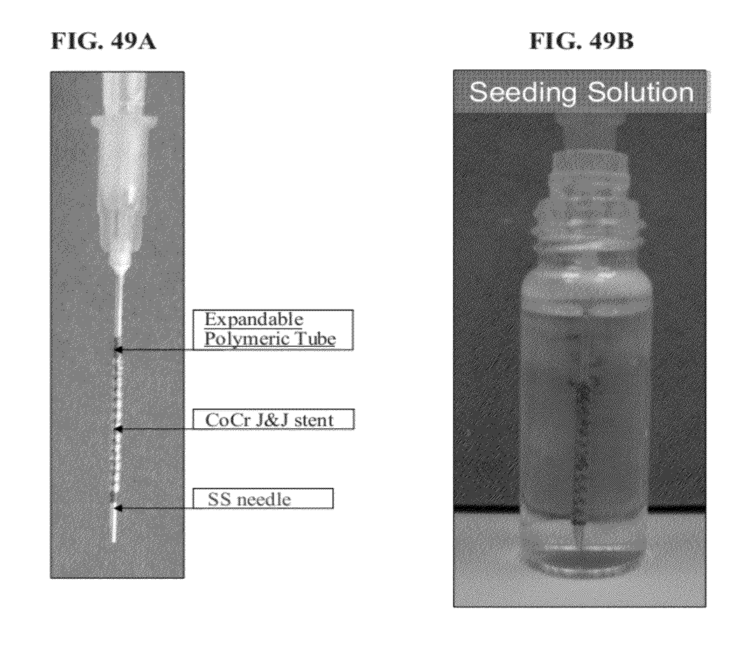

FIG. 49 presents an exemplary system for depositing crystalline drug only on the outer portion of a surface.

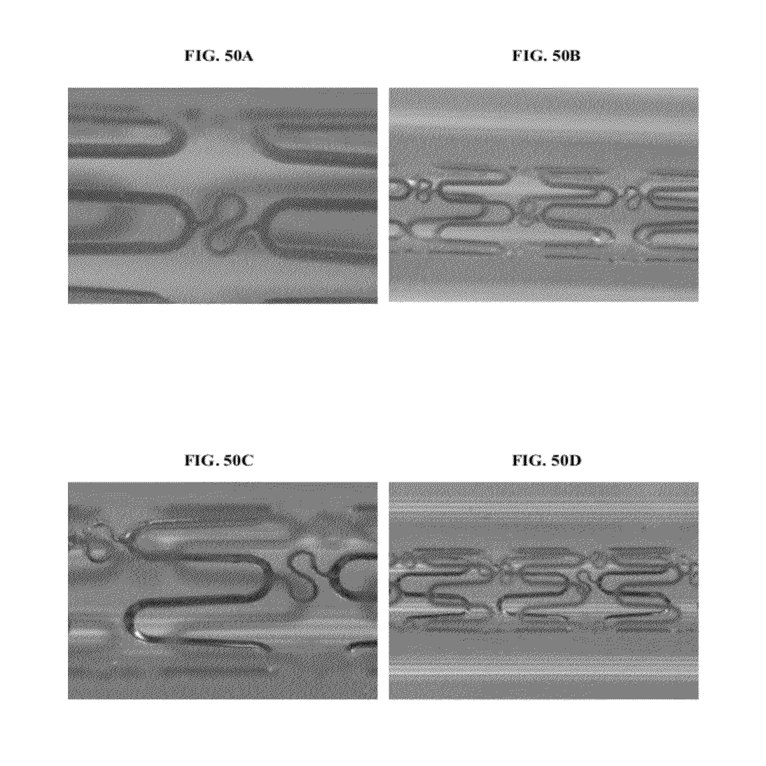

FIGS. 50-52 present images showing masking of surfaces which prevent crystallization on a portion of the surface.

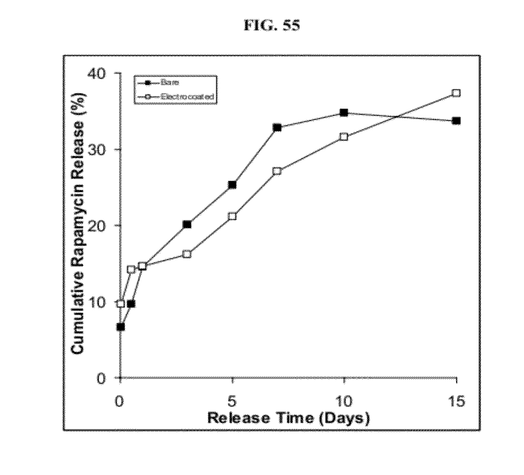

FIG. 54 presents images showing rapamycin crystals attached to the surface of a stent coated with a polymer.

Thus, according to one aspect of embodiments of the present invention, there is provided an article-of-manufacturing comprising an object having a surface and a therapeutically active agent being deposited onto at least a part of the surface, such that at least a portion of the therapeutically active agent that is deposited on the surface is in a crystalline form thereof.

According to some embodiments of the invention, the object in the article-of-manufacturing can have various shapes, including, but not limited to, a rod, a tubular body, a plate and a screw.

The object and/or its surface can be made of various materials. The object and the surface can be made from the same material or from different materials. Each of the object and its surface can independently be made of a polymeric material, a ceramic material, a glass, or a metallic material, including metal oxides.

The object and/or its surface can further be made from a biodegradable material or a biostable (non-biodegradable) material, depending on the intended use of the obtained article-of-manufacturing.

As used herein throughout, the term “biodegradable” describes a feature of a material that renders the material susceptible to degradation when exposed to physiological conditions. Thus, a biodegradable material (or compound) can decompose under physiological conditions into breakdown products. Such physiological conditions include, for example, hydrolysis (decomposition via hydrolytic cleavage), enzymatic catalysis (enzymatic degradation), and mechanical interactions.

The term “biodegradable” as used in the context of the present embodiments, also encompasses the term “bioresorbable”, which describes a substance that decompose under physiological conditions to break down to products that undergo bioresorption into the host-organism, namely, become metabolites of the biochemical systems of the host-organism.

A biodegradable material can decompose under physiological conditions during various time periods, ranging, for example, from a few hours to a few months and even a few years.

The term “biostable” or “non-biodegradable”, as used herein, describes a material that remains substantially intact under physiological conditions, as described hereinabove, and thus, does not undergo decomposition or degradation under these conditions.

The object and/or its surface can be made from a conductive, semi-conductive or non-conductive material.

Unless otherwise indicated, the term “conductive” relates to electric conductivity of a material, object or surface.

In some embodiments, the object's surface is made from a conductive or semi-conductive material, such that, for example, application of a base layer thereon can be effected via electroattachment, as detailed hereinbelow.

Suitable conductive surfaces for use in the context of some embodiments of the invention include, without limitation, surfaces made of one or more metals or metal alloys. The metal can be, for example, iron, steel, stainless steel, titanium, nickel, tantalum, platinum, gold, silver, copper, chromium, cobalt, any alloys thereof and any combination thereof. Other suitable conductive surfaces include, for example, shape memory alloys, super elastic alloys, aluminum oxide, MP35N, elgiloy, haynes 25, stellite, pyrolytic carbon and silver carbon.

In some embodiments, the object and/or its surface are made from a thermally conductive material. As described in detail hereinbelow, such a thermal conductivity of the object and/or its surface facilitates the process utilized for depositing on the surface a crystalline form of the therapeutically active agent, which involves a formation of a temperature gradient between the object and/or its surface and its surrounding.

In some embodiments, the object is a medical device.

The medical device can be used for implantation, injection, or otherwise placed totally or partially within the body, and hence it is desirable that the device will be a drug-eluting device.

In some embodiments, the medical device is for transdermal and/or topical applications in a subject. Such medical device should cause minimal tissue irritation when used to treat a given tissue and hence the inclusion of drugs therewith is beneficial.

Exemplary devices which can be used for transdermal application include, without limitation, a suture, an adhesive plaster and a skin patch.

Exemplary devices which can be used for topical application include, without limitation, a suture, an adhesive strip, a bandage, an adhesive plaster, a wound dressing and a skin patch.

In some embodiments, the medical device is an implantable medical device, for being implanted in a bodily organ of a subject.

The phrase “implantable device” is used herein to describe any medical device that is placed within a bodily cavity for a prolonged (e.g., from a few hours, to a few years and even for lifetime) time period.

Exemplary implantable devices include, without limitation, a plate, a mesh, a screw, a pin, a tack, a rod, a suture anchor, aortic grafts, arterial tubing, artificial joints, blood oxygenator membranes, blood oxygenator tubing, bodily implants, catheters, dialysis membranes, drug delivery systems, endoprostheses, endotracheal tubes, guide wires, heart valves, intra-aortic balloons, pacemakers, pacemaker leads, stents, ultrafiltration membranes, vascular grafts, vascular tubing, venous tubing, wires, orthopedic implants, implantable diffusion pumps and injection ports.

Additional exemplary devices include an anastomosis clip or plug, a dental implant or device, an aortic aneurysm graft device, an atrioventricular shunt, a hemodialysis catheter, a bone-fracture healing device, a bone replacement device, a joint replacement device, a tissue regeneration device, a hemodialysis graft, an indwelling arterial catheter, an indwelling venous catheter, a needle, a patent foramen ovale septal closure device, a vascular stent, a tracheal stent, an esophageal stent, a urethral stent, a rectal stent, a stent graft, a suture, a thread, a tube, a vascular aneurysm occluder, a vascular clip, a vascular prosthetic filter, a vascular sheath and a drug delivery port, a venous valve and a wire.

Examples of bodily sites where a medical device can be implanted include, without limitation, skin, scalp, a dermal layer, an eye, an ear, a small intestines tissue, a large intestines tissue, a kidney, a pancreas, a liver, a digestive tract tissue or cavity, a respiratory tract tissue or cavity, a bone, a joint, a bone marrow tissue, a brain tissue or cavity, a mucosal membrane, a nasal membrane, the blood system, a blood vessel, a muscle, a pulmonary tissue or cavity, an abdominal tissue or cavity, an artery, a vein, a capillary, a heart, a heart cavity, a male reproductive organ, a female reproductive organ and a visceral organ.

In some embodiments, the implantable medical device is a stent. The stent can be of various types, shapes and materials. Any commercially available stent, presently or in the future, can be used according to embodiments of the invention. Optionally, a stent particularly designed or modified for the purposes of the present embodiments, can be used.

Exemplary stents include, but are not limited to, the Z, Palmaz, Medivent, Strecker, Tantalum and Nitinol stents.

Further exemplary stents include, but are not limited to, YUKON® micropore stainless steel 316 LVM stent, by Translumina, a CrCo (L605) stent, a stent that serves for manufacturing CYPHER®, a bare stainless steel stent manufactured by Johnson & Johnson, Conor stent (J&J) with drug cavities, as presented in www.res-technology.com/, MULTI-LINK ULTRA Coronary Stent by Abbott Vascular, ABSOLUTE 0.035 Biliary Self-Expanding Stent System by Abbott Vascular, Dynamic™ (Y) Stent by Boston Scientific, WallFlex® Duodenal Stent by Boston Scientific, and currently developed bioresorbable stents such as, for example, a magnesium-based stent by Biotronix.

In cases where the object is a medical device, as described herein, the article-of-manufacturing may further comprise a packaging material in which the object (having the therapeutically active agent deposited on its surface) is packaged, and the article-of-manufacturing can be identified in print, in or on the packaging material, for use in the treatment of a medical condition treatable by the medical device, as detailed hereinbelow.

Herein, the terms “drug”, “therapeutically active agent” “pharmaceutically active agent” and simply an “agent” or an “active agent” are used herein interchangeably and describe a compound or composition that exhibits a beneficial therapeutic effect when administered to a subject. Thus, a therapeutically active agent is any agent known in the medical arts to have a therapeutic effect, and that is capable of treating or preventing, as these terms are defined herein, a medical condition.

Therapeutically active agents that are suitable for use in the context of embodiments of the invention include, but are not limited to, anti-restenosis agents, anti-thrombogenic agents, anti-platelet agents, anti-coagulants, statins, toxins, antimicrobial agents, analgesics, anti-metabolic agents, vasoactive agents, vasodilators, prostaglandins, thrombin inhibitors, vitamins, cardiovascular agents, antibiotics, chemotherapeutic agents, antioxidants, phospholipids, anti-proliferative agents, paclitaxel, rapamycin, and any combination thereof.

Additional agents include, but are not limited to, peptides, proteins, hormones, growth factors, enzymes, antibodies, nucleic acids, oligonucleotides, antisenses, and the like.

In general, the therapeutically active agent is such that can adapt a crystalline form under common crystallization or re-crystallization conditions (e.g., dissolution and cooling; crystallization from a supersaturated solution of the active agent dissolved in a methastable solution), whereby the crystalline form thereof is identifiable by common techniques, as delineated hereinabove.

In some embodiments, the therapeutically active agent is an anti-proliferative agent, such as, for example, those currently used in drug-eluting stents.

Thus, in some embodiments, the therapeutically active agent is a drug such as rapamycin or paclitaxel, including derivatives and analogs thereof.

In some embodiments, the therapeutically active is rapamycin.

The therapeutically active agent selected will typically depend on the intended use of the object. Thus, for example, paclitaxel and rapamycin are particularly suitable for certain implantable medical devices (e.g., stents).

As discussed hereinabove, at least a portion of the therapeutically active agent is in a crystalline form of the agent.

As used herein, the phrases “crystalline form”, “crystallized” and any other grammatical deviation thereof, referring to a therapeutically active agent or a drug, are used interchangeably and describe a form of a solid or semi-solid matter in which the constituent atoms and/or molecules are arranged in a 3-dimensional ordered, repeating pattern. The pattern can be detected according to known methods used in the chemical arts, including, for example, visual identification of crystals (typically by their relatively simple geometric shapes) and identification of X-ray diffraction patterns.

Thus, according to embodiments of the invention, at least about 20% of the agent on the surface of the object is preferably in a crystalline form. Optionally, at least about, 30%, 40%, 50%, 60%, 70%, optionally at least about 75%, optionally at least about 80%, 90%, optionally at least about 95%, and optionally at least about 99% of the agent on the surface is in a crystalline form. The degree of crystallinity may be determined according to any suitable method known to those skilled in the chemical arts, for example, to method described in Wang et al. [Am. J. Biochem. Biotech. 1:207-211, 2005].

The portion of the therapeutically active agent that is not in a crystalline form is in an amorphous form.

In some embodiments, at least 90% of the therapeutically active agent is in a crystalline form. In some embodiments, at least 99%, and even 100%, of the therapeutically active is in a crystalline form.

The crystalline form of the therapeutically active agent can be a single crystalline form, namely, a single isomorph (also referred to in the art as polymorph). Alternatively, the crystalline form of the therapeutically active agent can be polymorphic, namely, comprised of a number of isomorphs (or polymorphs).

As delineated hereinabove, one of the main advantages resulting from the novel methodology presented herein is the possibility and feasibility of depositing a therapeutically active agent on an object's surface, while controlling the release profile of the agent, without using a polymeric carrier for carrying a drug.

Thus, according to some embodiments of the invention, the article-of-manufacturing described herein is devoid of a polymeric carrier for carrying the therapeutically active agent.

As used herein, the term “carrier” describes a substance, typically a solid or semi-solid substance, which is deposited on the object's surface, and in which a drug is dispersed, embedded or encapsulated. Carriers are typically used to promote adherence of the therapeutically active agent to the surface and/or to control (e.g., to slow) the release of the therapeutically active agent.

A “polymeric carrier” refers herein to a carrier that comprises a polymeric material.

The absence of a carrier (e.g., polymeric carriers) in embodiments of the present invention may prevent deleterious effects of the carrier, which may result, for example, from the carrier peeling or flaking off of the surface. Thus, for example, in implanted objects carriers may lead to thrombosis, loss of control of drug release, distal embolization, and/or a delayed or abnormal endothelialization.

According to some embodiments, the therapeutically active agent is applied directly onto the surface.

In these and other embodiments, the surface is selected capable of inducing, facilitating, promoting and/or enhancing crystallization of the agent. For example, a surface containing numerous pores and/or cracks and/or impurities, which act as nucleation sites for crystallization may be used. Crystallization may also be facilitated with a surface which has an affinity to the therapeutically active agent. Such an affinity can also be used for improving an adherence of the therapeutically active agent to the surface.

In general, in these embodiments, a suitable surface is such that enables the formation of a local irregularity therewithin. Such local irregularities can thus be formed by physical irregularities, such as the pores and/or cracks and/or impurities described hereinabove, or by a change in the local temperature of these irregularities. These local irregularities can serve as nucleation sites for inducing crystallization of the therapeutically active agent.

According to other embodiments of the invention, the article of manufacturing further comprises a base layer, being applied onto at least a portion of the surface, such that the therapeutically active agent is deposited onto the base layer, rather than directly onto the surface. Preferably, the base layer has a sufficiently strong affinity to the surface, so as not to become detached from the surface. For example, the layer may be covalently bound to the surface or electrochemically attached to the surface.

Diazonium salts, and aryl diazonium salts (e.g., 4-(2-hydroxyethyl)phenyl diazonium salt, 4-dodecyloxyphenyl diazonium salt) in particular, are suitable for forming thin layers of moieties covalently bound to the surface. Optionally, the diazonium salt (e.g., aryl diazonium salt) is electrochemically attached to the surface, resulting in at least one moiety (e.g., an aryl moiety) attached to the surface, thereby forming a base layer. Exemplary base layers formed by electrochemically attaching diazonium salts to a surface, thereby forming a moiety attached to the surface, are disclosed in WO 08/090,554, which is incorporated by reference as if fully set forth herein.

In some embodiments, the base layer remains intact and attached to the object upon being subjected to physiological and/or mechanical conditions associated with the object for at least 30 days. The ability to withstand physiological conditions is of particular importance when the object is a medical device, even more so when the object is an implantable medical device.

In some embodiments, the base layer is very thin (e.g., a monolayer attached to the surface), such that any portion of the base layer which detaches from the surface has a very small volume, and hence, a minimal effect on its surroundings.

In some embodiments, the base layer is selected capable of inducing, promoting, facilitating and/or enhancing crystallization of the agent. For example, the base layer may form nucleation sites for crystallization of the selected therapeutically active agent. Thus, for example, the base layer can be selected capable of interacting with a functional group of the therapeutically active agent to be deposited thereon, so as to affect or induce a formation of crystalline form thereof. The base layer can further include an impurity or any other irregularity, as described hereinabove, that can serve as or form a nucleation site for crystallization.

In some embodiments, the base layer can comprise crystal seeds which are suitable for inducing crystallization of the therapeutically active agent. In some embodiments, the base layer comprises crystal seeds of the therapeutically active agent to be deposited. As demonstrated in the Examples section that follows, seeding of rapamycin crystals prior to depositing a crystalline form of rapamycin on the surface successfully facilitated the deposition of uniform and well-adhered crystalline drug.

In some embodiments, the base layer has an affinity to the agent such that the agent adheres to the base layer more strongly than to the surface without the base layer. The base layer may interact with the agent by a hydrophobic interaction (e.g., the layer and agent are both hydrophobic), a hydrophilic or electrostatic interaction (e.g., the layer and agent are both polar, the layer and agent have opposite charge), a π-interaction and/or any combination thereof.

In some embodiments, the base layer serves a mechanical layer, for mechanically carrying the therapeutically active agent and have the agents adhered to the surface. Such a base layer can be made, for example, of a porous material, in which the agent dispersed or embedded. Exemplary porous materials include, but are not limited to, metals, semi-metals, ceramics, metal oxides and the like. Such a mechanical base layer can therefore serve for improving the adhesion of the therapeutically active agent to the surface and further for forming nucleation sites on the surface.

In some embodiments, the base layer is a hydrophobic layer. A hydrophobic layer is advantageous as it prevents the effect of an aqueous environment on the adherence of the active agent to the surface and on the release of the active agent from the surface. In cases where the surface is hydrophilic in nature, diffusion of water molecules into the interface between the surface and the therapeutically active agent can be effected, resulting in reduced adherence and accelerated release of the active agent. Applying a hydrophobic layer onto a surface prevents such an effect and allows improved adherence and controllable release of the agent.

Exemplary materials that are suitable for forming a hydrophobic layer include, but are not limited to, aryls (which can be formed by electroattaching aryl diazonium salts, as detailed herein), fatty acid, and any other hydrophobic materials that can adhere to the surface and to the therapeutically active agent.

In some embodiments, the base layer comprises a polymer, for example, a hydrophobic polymer, a biostable polymer and/or a biodegradable polymer. An exemplary polymer is poly(D,L-lactic-co-glycolic acid), as described in Example 24 in the Examples section that follows.

It is noted that utilizing a polymeric base layer, according to embodiments of the invention, serves for, for example, forming nucleation sites on the surface, and for improving properties of the obtained article, such as flexibility, biocompatibility, hydrophobicity, and the like. The use of a polymeric base layer therefore differs from polymeric materials that are currently used as drug carriers in drug-eluting stents, which are aimed at providing a controllable release of the drug and enhanced chemical stability thereof.

It is further noted that while in currently used in drug-eluting stents, a polymeric carrier having a drug dispersed and/or embedded therein is deposited on the stent surface, according to embodiments of the invention, a base layer is first deposited on the object's surface and a crystalline form of the active agent is thereafter deposited on the polymeric (or non-polymeric) base layer.

In some embodiments, the layer is formed from the material on the surface, rather than from compounds attached to the surface. Thus, for example, a metal oxide layer may be generated on a metal surface by oxidation of the metal surface. The formed metal oxide can serve both for improving the adhesion of the therapeutically active agent to the surface and/or for forming nucleation sites on the surface.

According to some embodiments, the base layer is selected such that it is by itself another therapeutically active agent, for example, an agent that imparts biocompatibility to the surface, a coating which inhibits deleterious biological responses (e.g., restenosis and/or an immune response), a matrix which promotes healing, and/or a layer of a drug (e.g., an agonist or antagonist of a protein, an anti-platelet agent, an anti-thrombotic agent, an anti-restenosis drug, etc.) bound to the surface. Such a base layer may serve for therapeutic purposes, after the therapeutically active agent deposited thereon is released. Fatty acid, heparin and any other drugs can be used for forming such a base layer.

The properties of the base layer may be determined by selecting an appropriate material or precursor material for forming the base layer. Thus, for example, when the layer is formed by electroattaching an aryl diazonium salt, the chemical properties of the layer are primarily determined by the chemical properties (e.g., hydrophobicity, charge) of the aryl moiety of the aryl diazonium salt. Thus, using a base layer prepared by electroattaching an aryl diazonium salt, as described hereinabove, enables to form a tailor-made base layer, which exhibits the desired characteristics, by selecting the appropriate aryl moiety from which the layer is prepared.

The base layer is optionally designed capable of controlling the kinetic parameters of a release (e.g., rate of release, size of an initial burst of release) of the agent from the object. The affinity of the base layer to the agent and/or object may be used to control these kinetic parameters.

As demonstrated in the Examples section hereinbelow, the release profile of a crystalline form of a therapeutically active agent is differently that of the amorphous form of the same therapeutically active agent. For example, a crystalline form is typically released more gradually than the amorphous form, and in a more linear manner. The degree of crystallinity of the agent can therefore determine the release profile of the agent from the surface of the object. As further demonstrated in the Examples section that follows, a crystalline form of a drug can exhibit a release profile that is substantially similar to that of the same drug, when dispersed in a polymeric carrier. As further demonstrated in the Examples section that follows, by controlling various parameters of the process of depositing a crystalline form of a drug on an object's surface, the release profile of the drug can be manipulated.

These results strengthen the underlined basis of the invention, according to which deposition of an amorphous form of a therapeutically active agent on an object's surface results in non-controllable release of the agent, due to its relatively fast dissolution, whereby deposition of a crystalline form of a therapeutically active agent allows controlling on the release profile of the agent.

Hence, according to some embodiments of the invention, less than 20% (by weight), of the agent on the object is released upon subjecting the object to physiological conditions for 24 hours. According to some embodiments, less than 50% of the agent is released upon subjecting the object to physiological conditions for 5 days. According to some embodiments, less than 70% of the agent is released upon subjecting the object to physiological conditions for 16 days.

Such a gradual release of the agent generally results in a relatively constant release for a relatively long period of time, which is typically beneficial, as a relatively constant amount of agent released daily can more effectively match the desired daily dose of the released therapeutically active agent.

According to some embodiments, the crystalline form of the agent is selected such that an average diameter of the crystals in a range of 2 to 200 microns.

As demonstrated in the Examples section that follows, the release profile of the therapeutically active agent depends, at least in part, on the size of the crystals. Thus, it has been shown, for example, that larger crystals are released more slowly than smaller crystals. The size of the crystals may be modulated by selecting appropriate crystallization conditions. For example, crystallization with few nucleation sites, and under conditions which do not favor nucleation (e.g., at relatively high temperature and/or low concentration of agent), as detailed hereinbelow, typically result in larger crystals.

In some embodiments, the average diameter of the crystals is in a range of 75 to 200 microns. Such crystals are released in a gradual manner, such that less than 30% of agent is released after 5 days under physiological conditions and/or less than 60% of the agent is released after 16 days under physiological conditions.

In some embodiments, the average diameter of the crystals of the agent is in a range of 2 to 75 microns. Such care released t more rapidly than larger crystals, e.g., in a range of 75 to 200 microns.

As delineated hereinabove, the therapeutically active agent is deposited on at least a portion of the object's surface. Thus, the agent can be deposited, continuously or discontinuously, on all the surface area or on a part or few parts of the surface area.

Accordingly, the therapeutically active agent can be deposited onto, for example, 10%, 20%, 30%, 40%, 50%, 60%, 70%, 80%, 90% or even 100% of the surface area.

Similarly, the base layer, if present, can be applied onto, for example, 10%, 20%, 30%, 40%, 50%, 60%, 70%, 80%, 90% or even 100% of the surface area.

In some embodiments, the surface area onto which the base layer is applied at least overlaps the portion of the surface onto which the therapeutically active agent is deposited, but can be larger.

In some embodiments, a portion of a surface onto which the therapeutically active agent is deposited may optionally be free of the therapeutically active agent. Such an embodiment may be obtained, for example, by temporarily masking the portion of the surface when the agent is applied, or by removing the agent from the portion of the surface.

In some embodiments, the therapeutically active agent forms a continuous layer on the surface of the object. Such a continuous layer may be beneficial in that a relatively homogeneous coating of the surface is thereby formed.

In other embodiments, the therapeutically active agent forms a discontinuous layer on the surface of the object. A discontinuous layer may be beneficial in that it is less vulnerable to mechanical failure (e.g., cracking, breaking, peeling off) than a continuous layer, particularly in cases where the object is subjected to mechanical manipulations, such as expansion, or other mechanical stresses. A discontinuous layer can be obtained by controlling process parameters such as crystal's density, as detailed hereinbelow, by temporarily masking some portions of the surface when the agent is applied, or by removing the agent from portions of the surface.

The object may have an outer surface as well as an inner surface, for example, when the object has a tubular shape or any other hollowed shape. The therapeutically active agent may optionally be applied, exclusively or in part, onto the outer surface of the object. Such embodiments are beneficial, for example, in cases where the object is a medical device such as a stent. Such embodiments can be achieved by temporarily masking the inner portion of the surface when the agent is applied, or by removing the agent from the inner portion of the surface.

Masking of a portion or portions of the surface can be affected by any of the methodologies known in the art.

In some embodiments, the article of manufacturing further comprises a top layer, which is applied onto at least a portion of the therapeutically active agent and/or of the surface. Such a top layer is also referred to herein as a “top coating” or “top coat”.

Thus, the top layer can be applied onto all those portions of the surface onto which the active agent is deposited. Alternatively, the top layer is applied onto the entire surface, or, for example, onto the outer or inner portion of the surface.

In some embodiments, the top layer serves for temporarily protecting the therapeutically active agent, and thus is designed such that is can be readily removed.

In some embodiments, it may be desirable to reduce friction between the surface of the object and a second surface which the object may be rubbed against, in order to protect the object (e.g., the crystalline agent) and/or the second surface. For examples, in cases where the object is a stent or any other implantable medical device, an outer surface of the device can be subjected to frictions during its implantation, as a result of rubbing against walls of blood vessels or against other bodily tissues and/or organs. Such frictions can lead to a mechanical scrapping of the deposited agent off the surface and hence to a premature release of a portion of the therapeutically active agent.

Hence, according to some embodiments, the top layer serves as a lubricant, for facilitating implantation or otherwise placing the article of manufacturing in its intended location.

In other embodiments, the top layer serves for protecting the deposited therapeutically active agent from undesired chemical interactions prior to placing it in a desired location, and/or during the manufacture and/or storage of the article of manufacturing.

In other embodiments, the top layer serves for further controlling the release profile of the active agent.

In further embodiments, the top layer serves for facilitating crimping of the object, particularly in cases where the object is an implantable device such as a stent. The top layer may contribute for dispersing the pressure formed during crimping and can further prevent the formation of cracks.

In some embodiments, the top layer is made of a water-soluble material, so as to allow its removal shortly after it is placed, for example, in a physiological environment.

A top layer made of a water-soluble material can serve as protection against forces associated with preparation (e.g., crimping processes), packaging, shipping and/or use of an object (e.g., a medical device), but dissolve after a brief period of time in an aqueous environment. Water-soluble gels (e.g., hydrogels) are exemplary water-soluble materials that can be used as a top layer, according to some embodiments of the invention.

The optimal rate of dissolution in an aqueous environment will depend on the particular use of the object. Optionally, for an object used in physiological conditions (e.g., a medical device or an implantable medical device) at least 20% (by weight) of the top layer is dissolved after 1 hour under physiological conditions. Optionally, the percentage of top layer which dissolves after 1 hour under physiological conditions is in a range of 20% to 90%, optionally 30% to 70%, and optionally 40% to 60% (by weight).

A top layer should typically have some elasticity and lack stickiness. For devices which are used at temperatures other than room temperature (e.g., implantable medical devices used in the body), the top layer may be more resilient and less elastic at room temperature in order to more effectively protect the object before use, and more elastic and less resilient at the temperature at which the object is used (e.g., 37° C.) in order to ease the use of the object and facilitate the removal of the top layer after it is no longer needed.

One of ordinary skill in the art will be capable of selecting a material for forming a top layer with the appropriate chemical properties discussed hereinabove.

The top layer can be formed from, for example, biodegradable, hydrophobic, amphiphilic or hydrophilic polymers, from organic compounds such as fatty acids and glycerol, from surfactants such as TWEENS, and from any combination thereof.

Exemplary water-soluble materials for forming the top layer include, without limitation, a fatty acid, a lipid, a polyethylene glycol, poly(ethylene-vinyl acetate), poly(butyl methacrylate), poly(styrene-isobutylene-styrene), poly-L-lactide, poly-c-caprolactone, polysaccharide, carboxymethyl cellulose (CMC), dextran, chitosan, glycerol, gelatin, serum albumin, polyvinylpyrrolidone (PVP), arabinogalactan, EUDRAGIT®, an elastic polymer, and a gel (e.g., a hydrogel), as well as copolymers of the aforementioned polymers, and mixtures thereof.

It is noted that while the top layer may be made of certain polymeric materials, these polymeric materials do not serve as polymeric carriers for carrying the therapeutically active agent, but rather are used to be applied onto a therapeutically active agent already deposited on the surface. In addition, the polymeric materials utilized for forming the top layer are preferably characterized as water-soluble, as detailed hereinabove, and hence are not suitable for serving as drug-carriers.

The preparation of articles of manufacturing having a crystalline form of a therapeutically active agent deposited on a surface thereof, as well as of the uniquely designed articles of manufacturing presented herein, can be achieved by utilizing a novel methodology for depositing a crystalline form of a drug onto surfaces of various objects, as follows.

The present inventors have developed novel processes for producing articles-of-manufacturing, such as those described herein, which comprise a crystalline form of a therapeutically active agent.

Hence, according to another aspect of embodiments of the present invention, there is provided a process of preparing the article-of-manufacturing described hereinabove, or any other article on manufacturing having deposited on a surface of an object therein a crystalline form of an agent. The process is effected by contacting a surface of an object, as described herein, with a solution containing the therapeutically active agent, as described herein, and cooling the surface to a temperature below the temperature of the solution, so as to form a crystalline form of the agent on the surface, as described herein.

It is to be appreciated that typically, neither the solution nor the surface will have a single temperature, but rather a temperature gradient will be present. Thus, the phrase “cooling the surface to a temperature below the temperature of the solution” refers to an average temperature of the surface being lower than an average temperature of the solution.

In some embodiments, the solution containing the therapeutically active agent is saturated or supersaturated at the temperature of the solution. It is, however, sufficient for the solution to have a concentration of the agent which would result in a supersaturated solution at the temperature to which the surface of the object is cooled, in order for the agent to be deposited on the surface.

As used herein, the term “saturated”, with respect to the solution, describes the most concentrated solution possible at a given temperature.

The term “supersaturated” describes a solution that is more concentrated than normally possible and which therefore is not in equilibrium (a methastable solution).

A saturated or supersaturated solution may be prepared by adding thereto an anti-solvent of the agent. As used herein, the term “anti-solvent” describes a compound or mixture of compounds which, when added to a solution containing the agent, reduces the solubility of the agent in the solution.

In some embodiments, the anti-solvent may be added to the solution before contacting the solution with the surface of the object, so as to effect a process which is also referred to herein as “static” crystallization.

In some embodiments, the anti-solvent is added gradually to the solution after the solution is contacted with the surface of the object, so as to effect a process which is also referred to herein as “dynamic” crystallization.

The deposition of the agent on the surface is driven by the concentration of the agent and the degree of cooling of the surface of the object (namely, the temperatures gradient between the solution and the surface). As exemplified hereinbelow, the degree of the driving force (e.g., cooling and concentration) affects the nature of the deposition.

Thus, according to some embodiments, the degree of cooling and concentration of the agent are selected such that at least a portion of the agent is deposited on the surface of the object in a crystalline form.

According to other embodiments, the degree of cooling and concentration of the agent are selected such that at least a portion of the agent is deposited on the surface in a non-crystalline form. In these embodiments, the process further comprises raising the temperature of the object's surface having the non-crystalline form of the agent deposited thereon, while being in contact with the solution containing the agent, such that the non-crystalline form of the agent is converted to a crystalline form.

It is to be appreciated that deposition of a non-crystalline form is typically a result of a higher driving force of deposition (e.g., higher concentration of the agent and/or lower temperature of the surface) than is deposition of a crystalline form.

Further, it is to be appreciated that the extent of a crystalline form of the therapeutically active agent can be as defined hereinabove, and is further controllable by the process parameters described herein.

Thus, according to embodiments of the invention, the process described herein can be effected such that, for example, a concentration of the active agent in the solution, the temperature gradient between the solution of the object's surface and/or the time of contacting the solution and the surface at a certain temperature gradient, affect the degree of crystallinity (e.g., the portion of the therapeutically active agent that is in its crystalline form), the size of the crystals, and as a result, the adherence of the therapeutically active agent to the surface and/or the release profile thereof.

As exemplified hereinbelow, the formation of a crystalline form of the agent on the surface, by either direct deposition of a crystalline form or by conversion of a non-crystalline form to a crystalline form, is considerably enhanced by seeding the surface with small (e.g., in a range of about 50 nm to about 5 microns in diameter) crystals (crystal seeds) of the agent prior to contacting the surface with the solution containing the agent and the resulting deposition of the agent. In an exemplary embodiment, seeding is performed by sonicating the object in a dispersion containing the small crystals.

Seeding increases the number of crystals deposited on the surface and the degree to which the surface is covered by the crystalline form of the agent. The size of the crystals is typically reduced by seeding. The number, density and size of crystals obtained by the process can therefore be controlled by seeding the surface with an appropriate density of the small crystals.

It is to be appreciated that by controlling the density of obtained crystals, it is possible to determine whether the obtained crystalline form of the agent will form a continuous or discontinuous layer, as described hereinabove.

The density of the seeding can be readily controlled by increasing or decreasing the concentration of the small crystals (crystal seeds) contacted with the surface of the object (e.g., by sonication) and/or the time during which the small crystals are contacted with the surface. The density can be determined by common analytical methods, such as, for example, scanning electron microscopy.

The present inventors have surprisingly uncovered that the cooling of the surface of the object relative to its surroundings (namely, effecting a higher temperature gradient between the solution and the surface) enhances adherence of the therapeutically active agent, in its crystalline form, to the surface.

Hence, according to an optional embodiment, the temperature (e.g., of the surface of the object and/or the solution) is selected so as to enhance an adherence of the crystalline form of the agent to the surface. This enhancement of adherence is highly advantageous, as it allows the production of a crystalline agent adhered to the surface without necessitating the use of a potentially harmful carrier.

Alternatively or additionally, the time of the crystallization process is selected so as to enhance an adherence of the crystalline agent to the surface. For example, because the density of the crystalline form increases during the crystallization, the time during which crystallization occurs can be selected such that the crystalline agent forms a less dense and even discontinuous layer, which typically has better adherence to the surface than a dense continuous layer. The continuity of the layer may also be reduced by reducing the driving force of deposition (e.g., reducing degree of cooling and/or concentration of agent in solution). Furthermore, the time during which the surface is cooled can be selected so as to facilitate the enhancement of adherence by the cooling stage.

Thus, as discussed hereinabove, both a continuous layer and a discontinuous layer may be obtained as desired by selecting appropriate temperatures, seeding density, concentration of agent and times of crystallization.

Accordingly, in an exemplary embodiment, a continuous layer of the deposited therapeutically active agent is obtained by depositing a dense layer (e.g., which covers more than 50% of the surface area) of crystal seeds and thereafter immersing the seeded surface in a solution containing a medium or high concentration of the active agent, while cooling for a prolonged time.

Alternatively, a continuous layer of the deposited therapeutically active agent is obtained by depositing a dense layer (e.g., which covers more than 50% of the surface area) of crystal seeds and thereafter immersing the seeded surface in a solution containing a high concentration of the active agent, cooling and then raising the temperature for prolog incubation time.

In other exemplary embodiments, a discontinuous layer of the deposited therapeutically active agent is obtained by depositing a sparse layer (e.g., which covers less than 30%, less than 20% or even less than 10% of the surface area) of crystal seeds and thereafter immersing the seeded surface in a solution containing a medium or high concentration of the active agent, while cooling for a limited time.

Alternatively, a discontinuous layer of the deposited therapeutically active agent is obtained by depositing a sparse layer (e.g., which covers less than 30%, less than 20% or even less than 10% of the surface area) of crystal seeds and thereafter immersing the seeded surface in a solution containing a high concentration of the active agent, cooling and then raising the temperature for limited period of incubation time (less than 2 hours; e.g., 30-40 minutes).

A discontinuous layer can be formed, however, also by masking portions of the surface, as detailed hereinbelow.

According to some embodiments, the surface of the object is selected capable of, or is pre-treated so as to be capable of, inducing, promoting, facilitating and/or enhancing crystallization of the agent. For example, a porous surface, as described hereinabove, which facilitates nucleation may be selected, or formed by pre-treatment of a non-porous surface. Alternatively or additionally, the surface may be selected or pre-treated so as to have an affinity to the agent, as described herein.

In some embodiments, the surface is pre-treated by applying therein the base layer described hereinabove (e.g., by electroattaching an aryl diazonium salt thereto).

The above process may optionally be modified such that crystalline agent is absent from a portion of the surface, for example, by masking the portion of the surface with a substance during the process. The masking substance (e.g., a gel) can preferably be applied and removed in a convenient manner (e.g., applied by contact, removed by rinsing). In one embodiment, the inner surface of a tube-shaped object is masked by inserting a substance (e.g., a polymer) which expands (e.g., as a result of contact with the solution of the agent) so as to fill the inner space of the object, thereby masking the inner surface thereof.

At the end of the process, the solution is removed from the surface of the object, and the surface is dried.

According to some embodiments, once a therapeutically active agent is deposited on the desired portion of the object's surface, a top layer, as described herein, is deposited.

As further exemplified below in the Examples section, the present inventors have developed an apparatus which facilitates the convenient application of a process for preparing an object having a crystalline form of a therapeutically active agent deposited onto the surface thereof, as described herein.

Hence, according to another aspect of the present invention there is provided an apparatus for preparing an object (e.g., a medical device, a stent) having a surface and a crystalline form of a therapeutically active agent being applied onto at least a portion of the surface, the apparatus comprising a rod supporting the object; a cooling mechanism which is in thermal communication with the rod, for cooling the rod; and a receptacle for holding a solution containing the therapeutically active agent, such that when the object is supported by the rod and the receptacle holds the solution containing the therapeutically active agent, at least a portion of the surface of the object is in fluid communication with the solution containing the therapeutically active agent.

In some embodiments, the rod is a hollow rod, and cooling the rod is effected by a cooling mechanism that comprises a coolant that flows through the hollow rod.

Such an apparatus can further comprise a device for cooling the coolant (e.g., a pump, a dyuar) and/or a device for causing the coolant to flow through the rod, such as a mechanical or manual pump. An exemplary such apparatus is presented in FIG. 2.

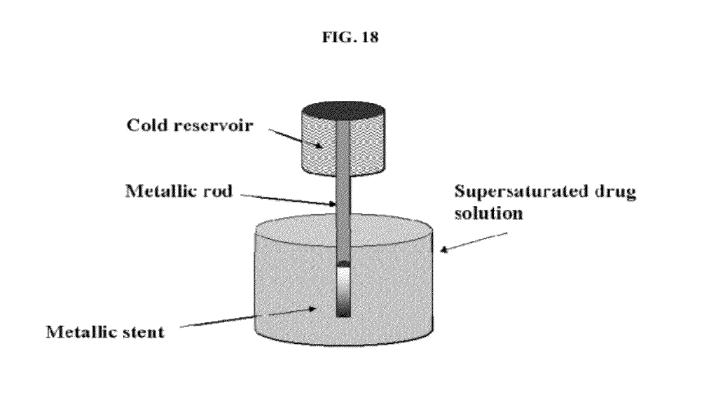

In some embodiments, the rod is a non-hollow rod, and cooling the rod is effected by a cooling mechanism that comprises a cooled reservoir, which is in direct communication with said rod. The cooled reservoir can include any means for forming the desired (low) temperature. The rod is cooled via thermal conductivity, as a result for being in direct contact with the cooled reservoir. As exemplary such apparatus is presented in FIG. 18.

As discussed herein, medical devices (e.g., implantable medical devices) prepared as described herein benefit from the advantageous properties of gradual release and controllable release profiles for the therapeutically active agent applied thereon.

Hence, according to another aspect of the present invention, there is provided a method of treating a subject having a medical condition in which implanting a medical device (e.g., a stent) is beneficial, which is effected by implanting a medical device as described herein within a desired bodily site of the subject. Medical conditions suitable for being treated by the aforementioned method include, without limitation, a cardiovascular disease, atherosclerosis, thrombosis, stenosis, restenosis, a cardiologic disease, a peripheral vascular disease, an orthopedic condition, a proliferative disease, an infectious disease, a transplantation-related disease, a degenerative disease, a cerebrovascular disease, a gastrointestinal disease, a hepatic disease, a neurological disease, an autoimmune disease, and an implant-related disease.

The therapeutically active agent and the device are selected suitable for treating the medical condition.

Accordingly, there is provided a use of the medical device as described herein in the treatment of a medical condition as described herein.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and the include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### MATERIALS AND EXPERIMENTAL METHODS

### Materials

All chemicals were used as received unless otherwise indicated.

CrCo (L605) stents (9 mm long; 1.3 mm diameter) were obtained from STI Laser Industries (Israel).

YUKON® micropore stainless steel 316 LVM stents (8 mm long; 1.4 mm diameter) were obtained from Translumina (Germany).

Bare stainless steel stents and CYPHER® drug eluting stents (18 mm long; 1.2 mm diameter) were obtained from Johnson & Johnson.

For some experiments, stents were expanded to a diameter of 3 mm, using a balloon catheter and a pressure of 10 atmospheres.

In all procedures, surfaces were electrocoated with a diazonium salt 4-(2-hydroxyethyl)phenyl diazonium tetrafluoroborate (DS-04) or 4-dodecyloxyphenyl diazonium tetrafluoroborate (DS-06), unless otherwise indicated. The diazonium salts were prepared by Elutex Ltd. in-house synthesis.

Electrochemical procedures were conducted using:

0.5 mm platinum wire (electrode), Holland-Moran Ltd., Israel [Catalog #Z730107];

20×10 mm platinum foil (electrode), Holland-Moran Ltd., Israel [Catalog #Z730204];

acetonitryl (99.9%, water <10 ppm), Holland-Moran, Israel [Catalog #326810010];

tetrabutylammonium tetrafluoroborate (TBATFB), Aldrich, Israel, [Catalog #217964];

ferrocene (98%), Sigma-Aldrich, Israel [Catalog #F408];

0.5 mm silver wire, Holland-Moran LTD., Israel, [Catalog #Z240099];

hydrobromic acid (48%), Fluka, Israel [catalog #18710].

Crystallization solutions were prepared from crystalline rapamycin (Sirolimus), Chunghwa Chemical Synthesis & BioTech CO., LTD., Taiwan [NDC 52076 6216]; ethyl acetate (EtAc) [bio-lab, lot 509451]; n-hexane [Bio-lab, lot 26176001], n-hexane (analytical), Frutarom, Israel [catalog #235554480].

For some experiments, rapamycin was recrystallized. 150 mg rapamycin (as received) was dissolved in 1.5 ml ethyl acetate and stored at 4° C. for 2 days. 30 ml of n-hexane were added to the rapamycin solution at a rate of 0.5 ml/minute. The solvents were evaporated under a chemical hood and the remaining residue was evaporated overnight using a vacuum pump.

Physiological environment was simulated using phosphate buffer, pH=7.7, 0.02% SDS. Phosphate buffer was prepared from sodium phosphate monobasic monohydride [Mallinckrodt AR® 7892 V10606 (ACS)] and disodium hydrogen phosphate dodecahydrate (Acros Organics, lot A0249582); sodium dodecyl sulfate [Bio Lab Ltd. Lot 541531, Catalog #19812323]; methanol (absolute), HPLC Supra gradient, [Bio-lab, lot 13683502]; and water (HPLC) (Bio-lab, lot 23210601).

Poly(tetrafluoroethylene) porous tubes (Catalog No. 0000027714) were obtained from Zeus (USA).

Carboxymethyl cellulose, sodium salt, 90,000 Daltons was obtained from Sigma (Cat. No. 419273).

### Methods:

### Electrochemical Processes:

Prior to the crystallization process, stents or stainless steel rods were electrocoated with a basic layer of DS-06 (4-dodecyloxyphenyl diazonium tetrafluoroborate) diazonium salt or DS-04 (4-(2-hydroxyethyl)phenyl diazonium tetrafluoroborate), unless stated otherwise. Before and after the electrocoating process, the stents were cleaned to remove impurities from the surface using sonication in acetonitrile for 15 minutes. Electrochemical processes and measurements were conducted using a Bio-Logic SA VSP potentiostat. A 3-electrode cylindrical cell was used, with the stent as working electrode (CrCo (L605) stents were held by CrCo (L605) wire, and stainless steel stents were held by stainless steel wire), platinum foil as counter electrode, and Ag/AgBr as reference electrode.

The electrocoating process was conducted by in-process cyclic voltammetry under a N₂ atmosphere inside a glove box. Each cell was bubbled with argon gas to eliminate O₂. Reduction of the diazonium salt was conducted by scanning from a potential of 0 V to −1.6 V vs. RE and back, at a scan rate of 100 mV/second. The scan was repeated for 30 cycles, resulting in an organic layer on the stent surface, followed by a decrease in current density, indicating blocking of the electrode (stent).

### Spray Coating:

Spray-coating was performed using a stent spray-coating device (Sono-Tek, USA). Following spray-coating, the entire stent was examined by light microscope to ensure no severe damage had occurred during preparation. Finally, stents were weighed using a micro-balance to determine coating weight.

The spray-coating solution for coating CrCo (L605) stents was 25 mg rapamycin dissolved in 8 ml ethyl acetate, except when stated otherwise, sprayed at rate of 0.05 ml/minute.

### Analyses:

### X-Ray Photon Spectroscopy (XPS):

XPS measurements were made on a Kratos Axis Ultra X-ray photoelectron spectrometer. Spectra were acquired with a monochromatic Al K 1486.7 eV X-ray source with a 0° takeoff angle. The pressure in the chamber was maintained at 1.5×10⁻⁹ torr during acquisition. The surveyed spectra were collected from 1200 to −5 eV (binding energy) with pass energy of 80 eV. High-resolution XPS scans were collected for C 1 s, O 1 s, Fe 2p, Cr 2p and Br 3d peaks with pass energy of 20 eV. Size leaps were 1 eV for survey scans and 0.1 eV for high-resolution spectra. XPS binding energy was calibrated to the peak position of Fe⁰ 2p_{3/2} as 707.0 eV. Data analysis and processing were performed with vision processing data reduction software by Kratos Analytical Ltd. and CasaXPS, by Casa Software Ltd.

### Scanning Electron Microscopy (SEM):

Scanning electron microscopy was conducted at the Technion (Haifa, Israel).

Images were taken by high-vacuum scanning electron microscope (Quanta 200 FEI, USA) and EDS Oxford Instruments energy-dispersive spectrometer INCA, attached to SEM, LEO Gemini 982, emission gun HR-SEM (FEG-SEM) which includes an in-lens detector for low voltage applications, an EDAX light-element EDS system, and an Oxford electron backscattered electron diffraction system (EBSD). Crystallized stents were plasma coated with a 10 nm gold deposition layer. Samples were characterized for their morphology, crystals size and size distribution (using an Image plus program).

### Light Microscopy:

An Olympus SZX16 light microscope with a CCD camera and Colorview software imaging system were used. The stent was placed under the lens, a 3300° K lamp was operated (Olympus KL2500), and images were taken at magnitudes ×1, ×2, ×4, ×8.

### Melting Point (m.p.) Measurements:

Measurement of the melting point of the drug enabled determination of whether the drug was amorphous or crystalline. The drug was placed in a m.p. capillary and melting point was measured in Stuart SMP10 instrument (UK). The sampled drug was taken from the crystalline coating layer on the stent and from walls of the vessel.

### Differential Scanning Calorimetry (DSC):

A Perkin Elmer TC-15 TE instrument was used.

In this thermo-analytical technique the difference in the amount of heat required to increase the temperature of a sample and a reference are measured as a function of temperature. Crystals were placed in a curve with 2 holes, an empty curve was used as background, and analysis was conducted between 25-300° C. at increments of 10° C./minute.

### X-Ray Diffraction (XRD):

X-ray powder diffraction measurements were performed on a D8 advance diffractometer (Bruker, AXS, Germany) with a gomiometer radius of 217.5 nm, Gobel mirror parallel beam optics, 2° Sollers slits and a 0.2 mm receiving slit. The powder samples were placed on low background quartz sample holders. XRD patterns from 20° to 60° (2θ) were recorded at room temperature using CuKα radiation (λ=0.15418 nm) with the following measurement conditions: tube voltage of 40 kV, tube current of 40 mA, step mode with size of 0.02° (2θ) and a counting time of 1 second per step. The instrumental broadening was determined using LaB₆ powder (NIST SRM 660).

The degree of crystallinity was calculated according to the method described in Wang et al. [Am. J. Biochem. Biotech. 1:207-211, 2005].

### Weight:

A Mettler Toledo MX5 instrument (d=1 μg) was used.

Stents were weighted before crystallization and afterwards. The difference in weights was taken as the weight of the drug deposited on the stent.

### Pharmacokinetics:

100 ml of phosphate buffer (pH 7.7) with 0.02% SDS (sodium dodecyl sulfate), was prepared from 0.0262 gram sodium phosphate monobasic monohydride, 0.289 gram disodium hydrogen phosphate dodecahydrate, and 100 μl of SDS solution (20%).

Unless stated otherwise, samples were incubated in 20 ml or 40 ml buffer, and small amounts (e.g., 1 ml) of the buffer were removed at each sampling time in order to measure released drug, and replaced with fresh buffer.

### HPLC Measurements:

The column was cleaned by flowing methanol for 1 hour at a flow rate of 1 ml/minute before and after running the samples. The mobile phase for detection was 90% methanol, 9.9% double-distilled water (DDW), 0.1% THF (tetrahydrofuran). Sample volume of 20 μl, wavelength of 278 nm, and flow rate of 0.4 ml/minute were applied.

A calibration curve was prepared using HPLC-obtained data of rapamycin solutions prepared at concentrations of 1, 2.5, 5, 7.5, 10, 12.5, 25, 50 and 100 μg/ml in phosphate buffer.

### Example 1

### Drug Deposition Kinetics in a Supersaturated Solution

Solutions with various quantities of rapamycin were prepared, and spontaneous sedimentation and/or crystallization onto the walls of the vessel was examined as a function of time at various temperatures.

0.1, 3 or 15 mg of rapamycin were dissolved in 1 ml of ethyl acetate, and 20 ml of n-hexane was then added slowly at a rate of 0.5 ml/minute using a syringe pump. The system was kept at a constant temperature (0, 15 or 30° C.). Aliquots of 1 ml were taken after 1, 3 and 6 hours and the concentration of rapamycin remaining in solution was determined by HPLC.

As shown in FIG. 1, the rate of deposition of crystalline rapamycin was highest at low temperature (i.e., 0° C.) for any given concentration, and at high concentration (i.e., 15 mg) of rapamycin for any given temperature.

These results indicate that low temperature and high concentration are driving forces for deposition.

### Example 2

### Static System for Deposition from a Supersaturated Solution

Rapamycin was dissolved in 1 ml of ethyl acetate (room temperature) in a small vial and removed to a glass tube located in an ice bath at 0° C. To this tube, 20 ml of n-hexane were added at a rate of 0.5 ml/minute using a syringe pump. The hexane was directed to the wall of the vessel to avoid droplet formation. The prepared solution was then transferred to a 40 ml chemical glass for deposition on a stent.

The stent was placed on a 2.7 cm long, 1.6 mm diameter, hollow stainless steel rod that was connected to a closed system of pipes. The pump passed coolant (n-hexane) at rate of 10 ml/minute from a vessel with the coolant, through the rod and back to the vessel. The pipes between the pump and the rod were placed in a Dewar flask with dry ice and acetone, at −78° C. The stent was first immersed in the pre-prepared solution and the pump was then operated. When the process time was finished, the pump was stopped and the stent was immediately removed from the solution, unless mentioned otherwise. During the process, the glass was covered in order to prevent entry of any impurities. The cooling system is shown schematically in FIG. 2.

Solutions were prepared at various concentrations of rapamycin, and categorized as high, medium or low concentration. As shown in Example 1, the concentration was found to affect the rate of deposition.

### Medium Concentration Solutions:

Solutions of 15 mg or 17.5 mg rapamycin dissolved in 1 ml ethyl acetate and 20 ml n-hexane were prepared at 0° C., and transferred immediately to the cooling system.

The stent holder on hollow stainless steel rod was then immersed in the solution while being cooled. The deposition process was conducted during 100-120 minutes, during which the solution remained clear, indicating that the drug did not aggregate in solution. The flow rate of the coolant was 10 ml/minute and the glass vessel containing the solution was open.

As shown in FIGS. 3A-3C, a uniform layer of drug was observed all over the stent surface.

The weight of rapamycin obtained on the stent and rod together was 230, 270 and 320 μg, respectively, for the samples shown in FIGS. 3A, 3B and 3C.

In an additional experiment, cooling of the solution was performed in addition to cooling of the stent. The rapamycin solution (15 mg rapamycin) was placed in an ice bath (ice with water) during the process, causing the clear pre-prepared solution to turn milky immediately upon immersion of the stent therein and flowing of the coolant. When the process was completed, the solution became clear with sunken aggregates.

As shown in FIG. 4, massive deposition of drug was observed by light microscope. The weight of the rapamycin on the stent in rod was 825 μg. At least part of the deposited rapamycin appeared to be the rapamycin aggregates observed in solution which then adhered to the surface.

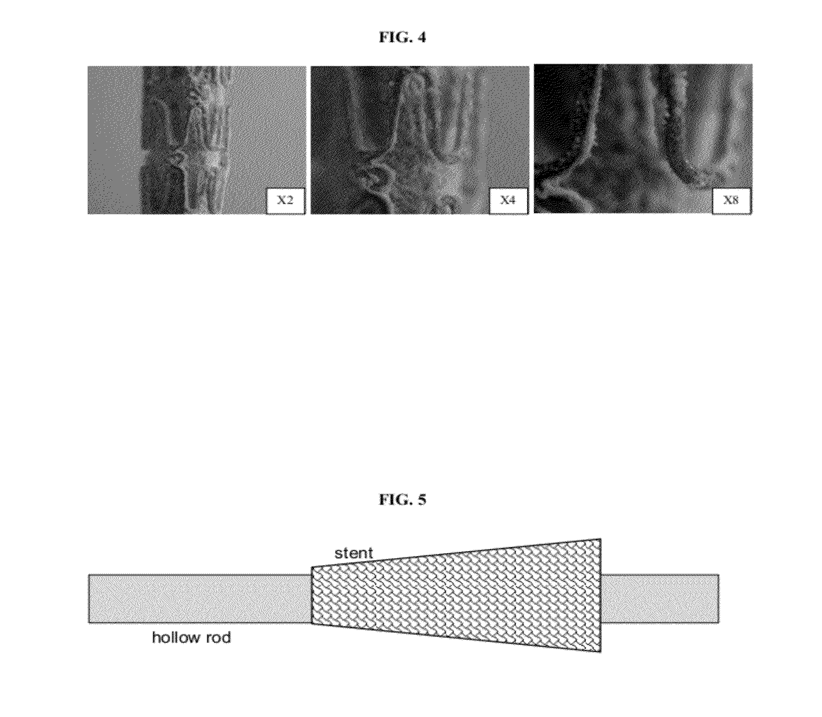

In other experiments, stents were widened in a conical manner, as shown in FIG. 5, such that the stent had only a small contact area with the stainless steel holder. The wider side reached 2 mm and 2.45 mm in diameter (as compared to the original diameter of 1.6 mm). The solution contained 15 mg rapamycin in the above-mentioned volumes, and was cooled in an ice bath as described above. The process was conducted for 100 minutes and the solution was surrounded with air.

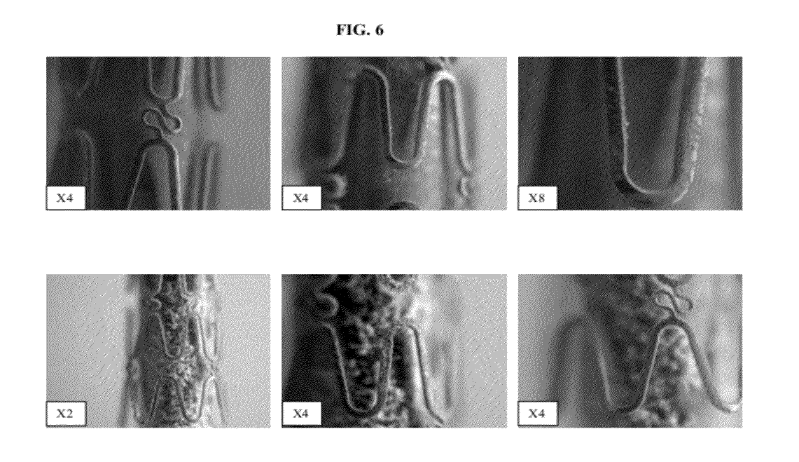

As shown in FIG. 6, the widened area of the stents exhibited uniform deposition with no dendrite growth. The solution remained clear throughout the process. The 2 mm wide sample had 1246 μg rapamycin and the 2.45 mm sample had 1047 μg of rapamycin deposited thereon.

### High Concentration Solutions:

Solutions of 25 mg rapamycin dissolved in 1 ml ethyl acetate and 20 ml n-hexane were prepared at 0° C. and were handled as described hereinabove. Deposition was conducted for 60 or 120 minutes and resulted in the stents being fully covered with a white deposition, visible by eye.

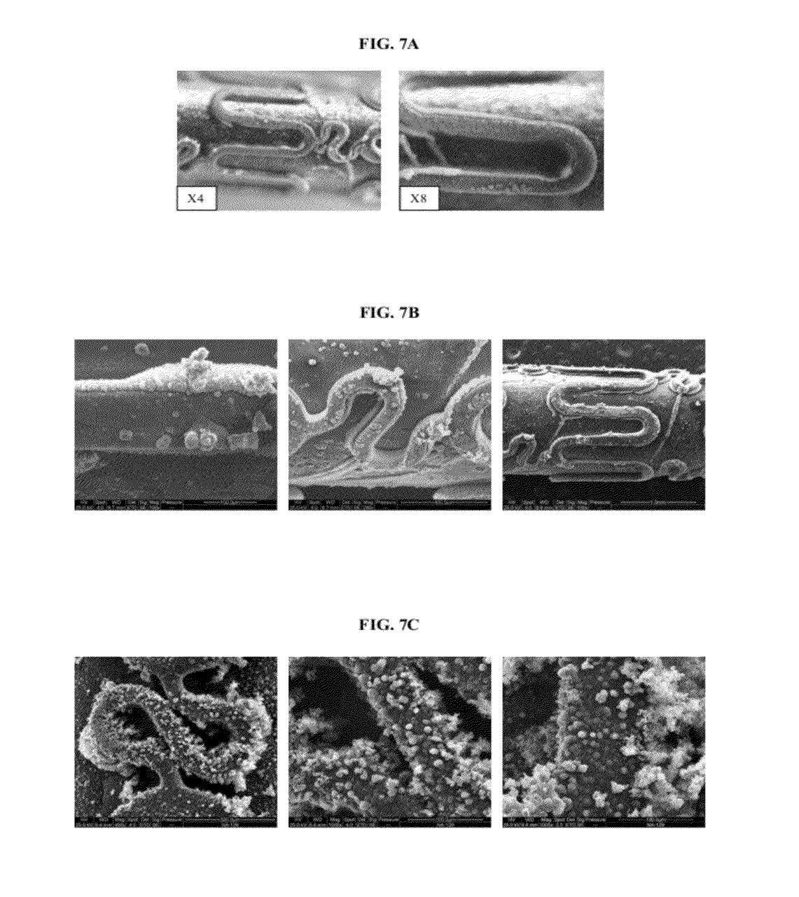

As shown in FIGS. 7A-7C, light microscopy and SEM images revealed a uniform deposition on the surface. The solution remained clear throughout the process.

In an additional experiment, deposition with 25 mg rapamycin was also conducted for 60 minutes as described hereinabove, and then stent cooling was stopped and the stent was kept immersed in solution overnight at room temperature. During stent cooling, the solution was placed in a water bath, which minimized cooling of the solution.

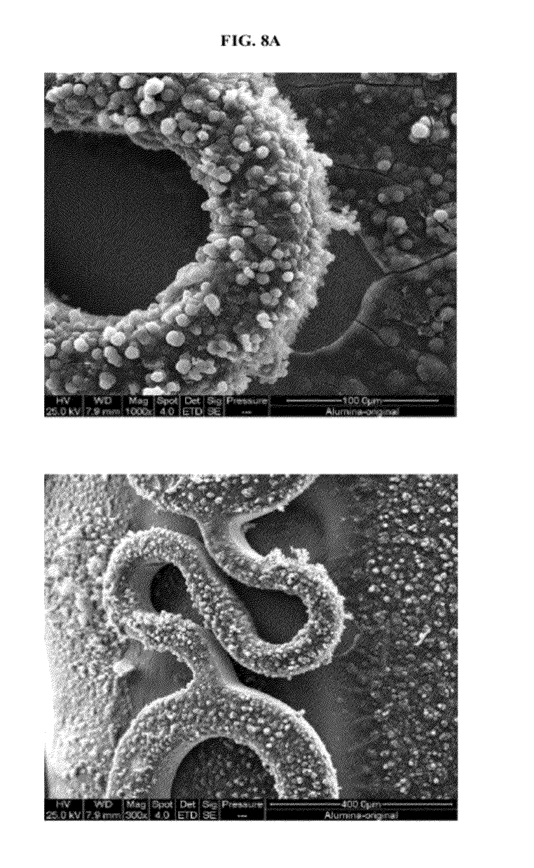

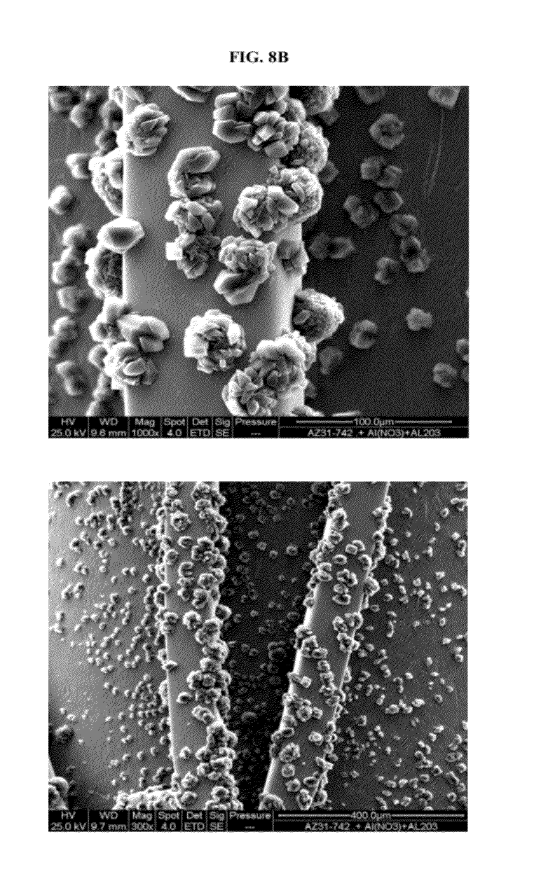

As shown in FIGS. 8A and 8B, minimizing the cooling of the solution and incubating the stent in the solution at room temperature following cooling of the stent for 60 minutes (930 μg drug weight) resulted in unevenly distributed crystals on the surface of the stent (FIG. 8B), whereby incubation for only 30 minutes resulted in a deposition which was lighter (440 μg) and lacked a clearly crystalline structure (FIG. 8A).

### Low Concentration Solutions:

Solutions of 5 and 10 mg rapamycin dissolved in 1 ml ethyl acetate and 20 ml n-hexane were prepared at 0° C. and handled as described hereinabove. Deposition was conducted for 120 minutes. Solutions remained clear during the process but almost no deposition was observed on the surface of the stent.

### Example 3

### Effect of Deposition Process on Crystallinity

The crystallinity of rapamycin deposited on CrCo stents was determined using X-ray diffraction (XRD). The results of four processes for depositing rapamycin were compared.

Process A:

Stents were seeded with rapamycin crystals crushed using a mortar and pestle, by sonicating the stent 3 times with 500 μg of the crushed crystals in 500 μl n-hexane for 5 minutes, with 1 minute intervals. A solution of 15 mg rapamycin in 1 ml ethyl acetate and 20 ml hexane was then deposited as described in Example 2 hereinabove, for 30 minutes, with a coolant flow rate of 10 ml/minute.

Process B:

1% (weight/volume) rapamycin dissolved in ethyl acetate was spray-coated onto the stent.

Process C:

Same as Process A, but followed by an additional incubation of the stent in the solution at room temperature for 120 minutes.

Process D:

Same as Process A, except that 3 mg rapamycin was used instead of 15 mg, coolant flow rate was 5 ml/minute, and deposition was for 60 minutes.

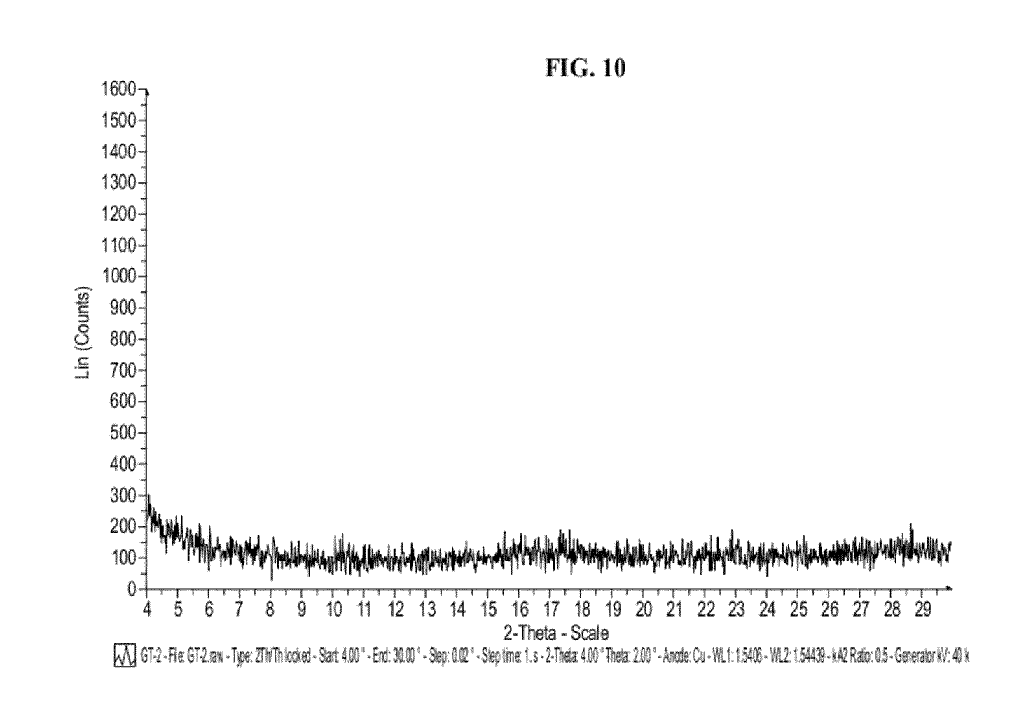

As shown in FIGS. 9 and 10, the deposited rapamycin obtained by both Process A and Process B was amorphous. The crystallinity of the deposited rapamycin was calculated to be 0%.

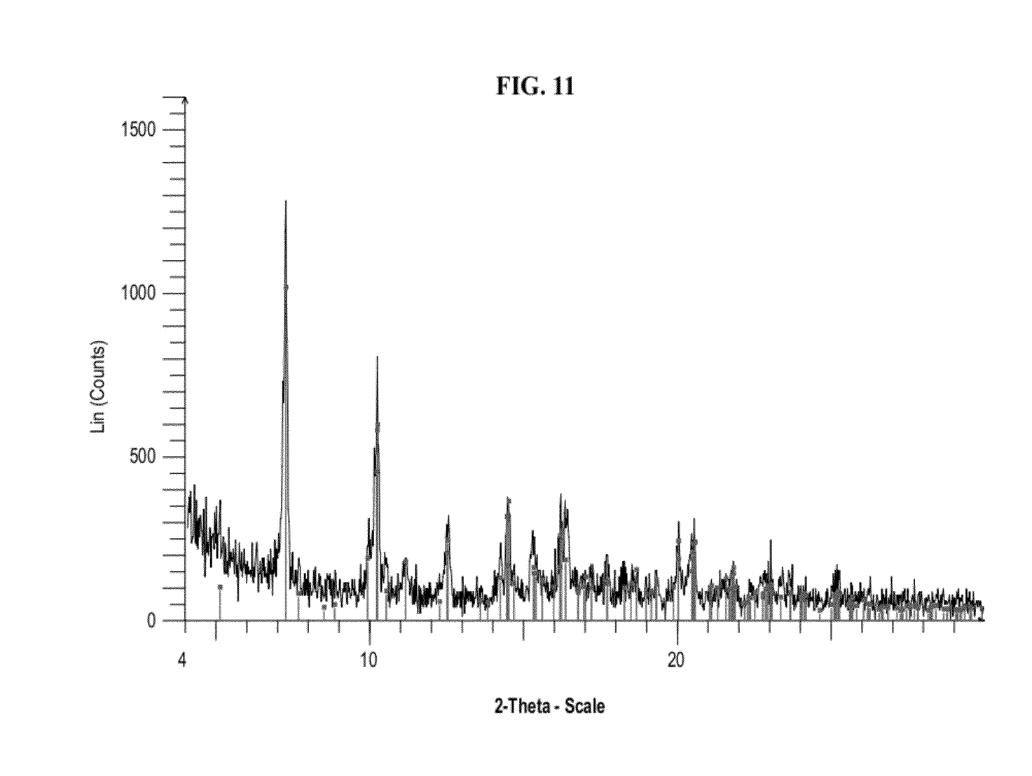

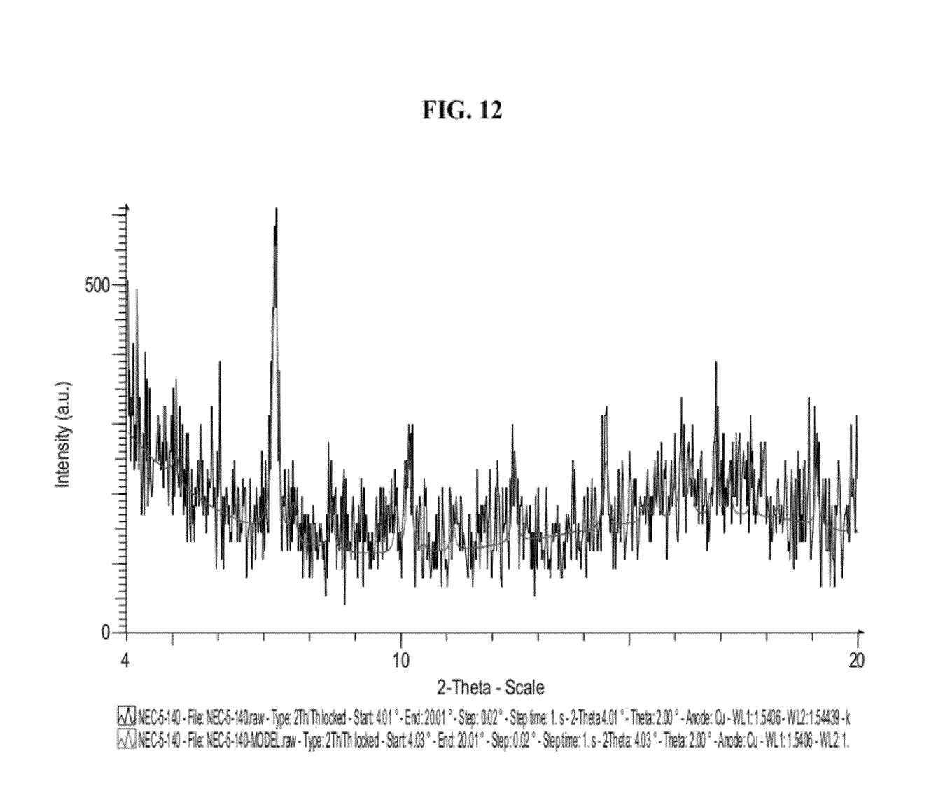

As further shown in FIGS. 11 and 12, the deposited rapamycin obtained by both Process C and Process D was crystalline. The crystallinity of the deposited rapamycin was calculated to be 100%. The observed crystalline form was an orthorhombic system (cell parameters: 34.8, 13.0, and 12.2 angstrom) matching the Type II isomorph of rapamycin (as described in U.S. Pat. No. 7,282,505).

These results indicate that while Process A results in an amorphous layer, incubation at room temperature (as in Process C) is capable of converting the amorphous layer into a crystalline layer. These results further indicate that use of a lower drug concentration and lower coolant flow rate (as in Process D) can result in direct crystalline deposition instead of amorphous deposition (which can be converted to a crystalline form).

### Example 4

### Dynamic System for Deposition from a Supersaturated Solution

The deposition of rapamycin onto stents was examined in a dynamic system, i.e., a system wherein the concentration of anti-solvent (n-hexane) and/or the temperature is non-constant during the deposition process.

CrCo (L605) stents were electrocoated with DS-06 (unless stated otherwise), as described hereinabove.

Rapamycin was dissolved in 1 ml of ethyl acetate at room temperature and cooled to 0° C. n-Hexane was then added at various rates and total amounts. The hexane was directed to the wall of the vessel to avoid droplet formation.

The stents were then placed on a stainless steel round wire which served as their holder, were immersed in the solvent (fully or partially, depending on the initial volume) and n-hexane was added using a syringe pump with an adjustable rate. A magnetic stirrer was used in part of the experiments, in order to obtain a homogeneous solution.

a) 25 mg rapamycin were dissolved in 1 ml ethyl acetate. The solution was cooled to 0° C. and placed in a glass tube. 3 CrCo (L605) stents were partially immersed in the solution at the beginning, and 25 ml n-hexane was added at a rate of 0.5 ml/minute, with hexane serving as an anti-solvent. No mixing of the solution was performed, thereby reducing the likelihood of aggregation in solution.

The solution remained clear during the process and no aggregation was observed in the solution. The system was kept for 64 hours at room temperature. 895, 682, and 592 μg of crystalline rapamycin was obtained on stents.

As shown in FIGS. 13A and 13B, most of the stent surface was bare, but massive growth of large crystals (−100 μm) was observed on the stents in a highly non-uniform manner. As shown in FIG. 13B, crystal growth was initiated at the stent surface.

An additional experiment was conducted with the same procedure, except that the stents were removed from the solution after 48 hours. 47, 23 and 70 μg of rapamycin, respectively, were obtained on the 3 stents.

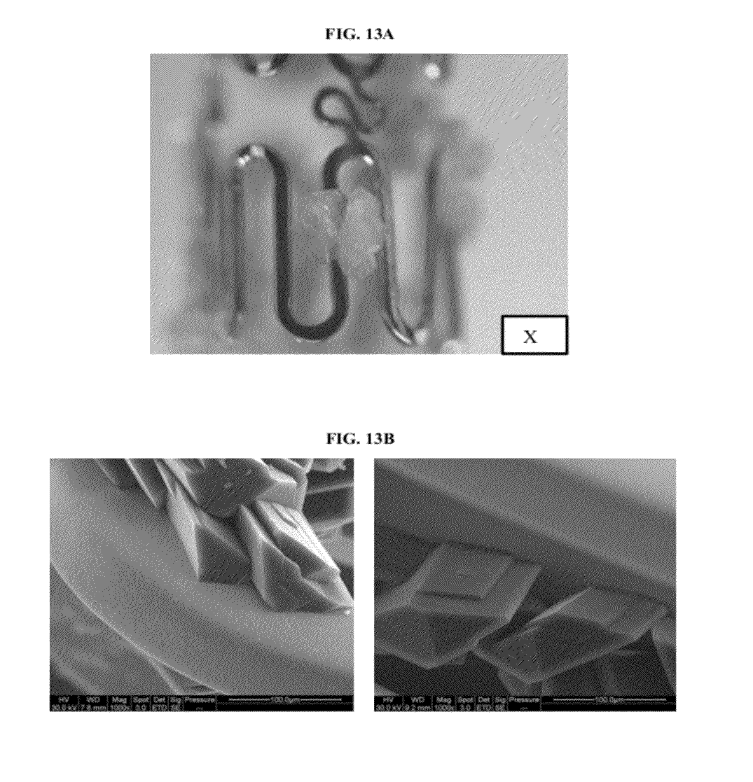

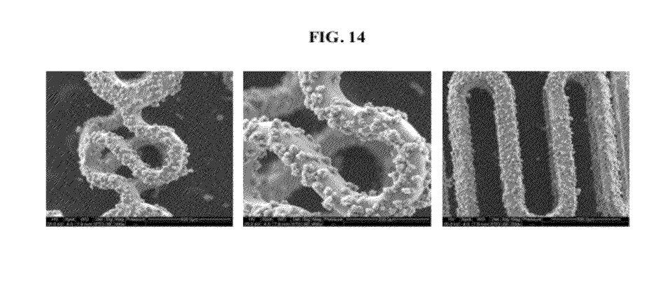

As shown in FIG. 14, crystals covered much of the stent surface, but considerable bare areas remained. Crystal size was 20-25 μm. The solution remained clear and crystals were also observed on the walls of the glass vessel.

b) In additional experiments, 100 mg rapamycin was dissolved in 4 ml ethyl acetate, stents were immersed in the solution and 22 ml n-hexane was added at a rate of 0.5 ml/minute, as described above. A magnetic stirrer was used to stir the solution. The solution became milky while being prepared, indicating aggregation of rapamycin in solution, and 13-20 μg of rapamycin was obtained on the stents.

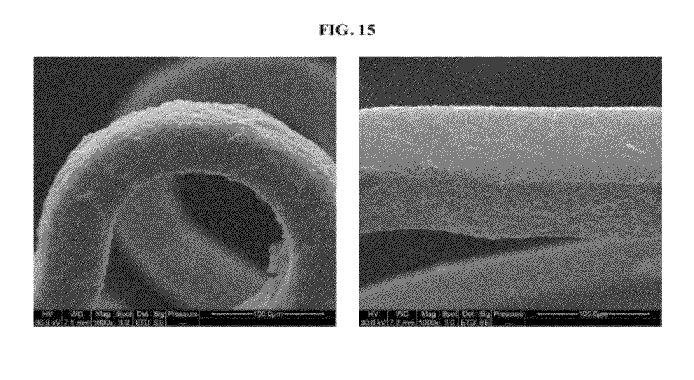

SEM images showing crystal deposition on the surface of the stent are presented in FIG. 15. Crystals were also observed on the walls of the glass tube. The melting point of these crystals was 192° C., as compared to 182° C. of standard (as received) rapamycin, indicating a high purity of the crystals.

c) 22 ml of n-hexane were added at rate of 0.2 ml/minute to 100 mg of rapamycin in 1 ml ethyl acetate at 0° C. A magnetic stirrer was used to stir the solution. The solution became milky during the process.

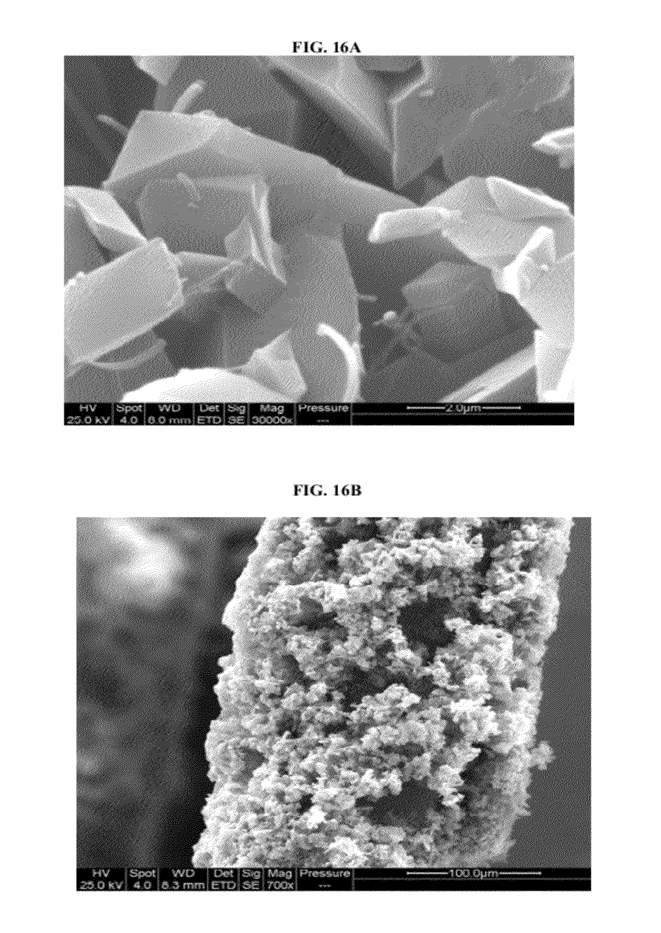

As shown in FIG. 16, a massive growth of crystals was observed on all 3 tested stents. Crystal size was 3 μm. 803, 1313 and 1158 μg of crystalline rapamycin, respectively, were obtained on the 3 stents. Crystals were also observed on the glass walls of the vessel.

d) Lowering the rapamycin concentration in the solution to 10 mg per ml of ethyl acetate resulted in a reduction of the weight of rapamycin deposition on the stents to 230, 260 and 455 μg. 20 ml n-hexane was added at a rate of 0.2 ml/minute, using also a normal size magnetic stirrer at slow speed.

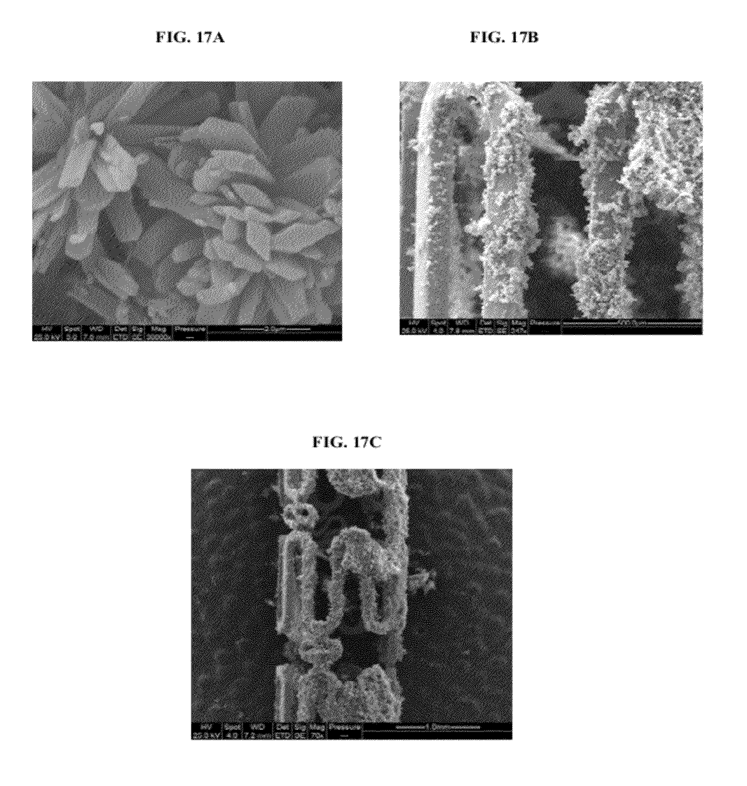

As shown in FIG. 17, non-uniform growth of crystals on the stents surface was observed. Crystal size was 2 μm. The obtained solution was clear.

e) A different system was examined, as presented schematically in FIG. 18.

The stent itself was cooled during the addition of the anti-solvent (n-hexane), using a cooling reservoir. The solution was prepared in a glass tube, where 100 mg rapamycin were dissolved in 4 ml of ethyl acetate at 0° C. n-Hexane was added at a rate of 0.5 ml/min through a tube connecting a syringe pump to the glass vessel. The tube carrying the n-hexane was immersed in a bath with dry ice and acetone. In addition, the stent was placed on a solid metal rod which was in contact with the bath of dry ice and acetone, such that the stent was cooled by the rod.

The stent was electrocoated with DS-04 before the deposition. 16 ml of n-hexane was added and the solution became milky.

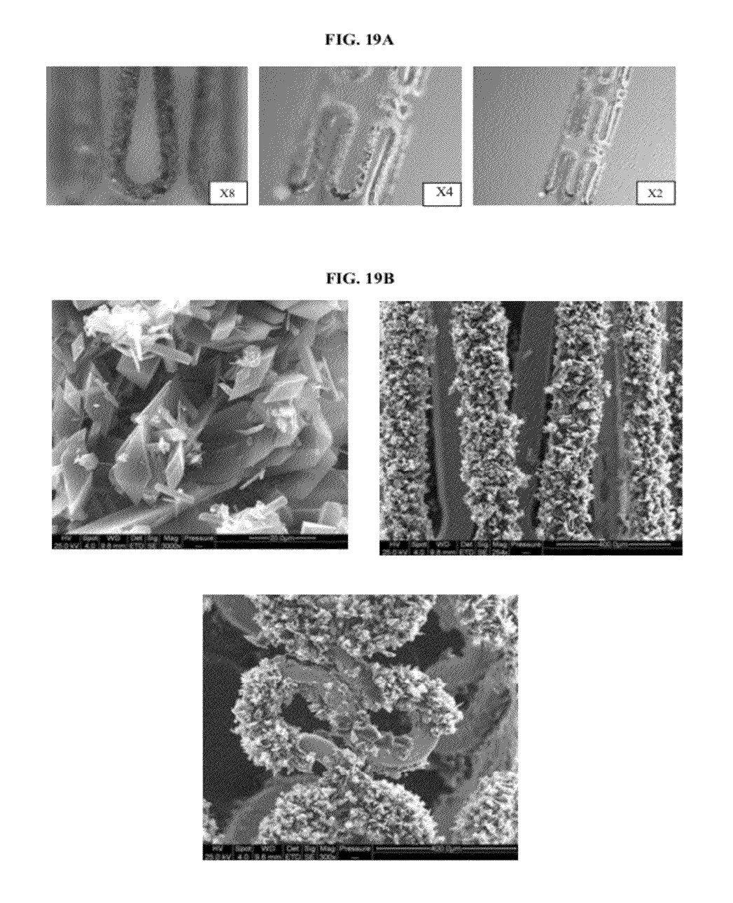

As shown in FIGS. 19A and 19B, crystals were observed all over the stent surface area, with a crystal size of 20-30 μm. 260 μg rapamycin was obtained on the stent.

### Example 5

### Effects of Seeding on Crystallization

CrCo (L605) stents and stainless steel stents and rods were electrocoated with DS-06, as described hereinabove.

YUKON® stainless steel stents (Translumina) were used. The surface of these stents consists of 10-100 μm² micropores which may facilitate population of the stent surface with rapamycin crystals by acting as nucleation sites. The surface of a bare YUKON® stent is shown in FIG. 20.

Crystals of re-crystallized rapamycin or rapamycin as received were ground and added to n-hexane, and gently shaken. Stents and rods were placed in the solution and the solution was subjected to sonication for three 1 minute periods with 1 minute intervals.

15 mg rapamycin was dissolved in 1 ml ethyl acetate. n-Hexane was added, and the stent was placed on a rod for crystallization at a low temperature, using a process similar to Process C as described hereinabove in Example 3.

The pump was operated for 30 minutes at a flow rate of 10 ml/minute. The stent was then removed from the solution and taken off the rod. The stent was placed on a new rod and was then placed back in the solution at room temperature for an incubation period of 2 hours (unless stated otherwise). The stent was then removed and dried at room temperature. As indicated in Example 3, crystalline rapamycin appears on the stent during the incubation at room temperature.

In some experiments, no stent was used. Instead, the rod connected to the pump was electrocoated, and crystallization on the rod was examined.

For comparison, some stents and rods were spray-coated with a solution of 25 mg rapamycin in 4 ml ethyl acetate at a rate of 0.05 ml/minute.

Stainless steel stents were seeded with re-crystallized rapamycin and then placed on a hollow stainless steel rod connected to the pump pipes.

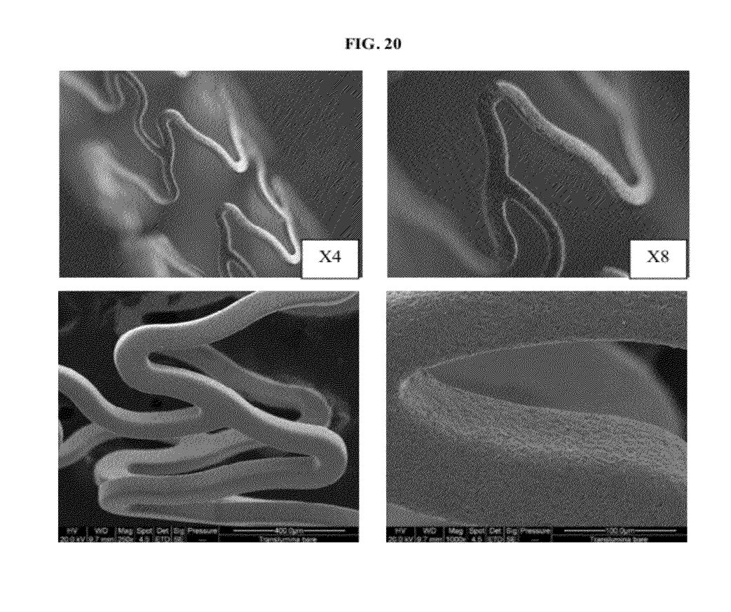

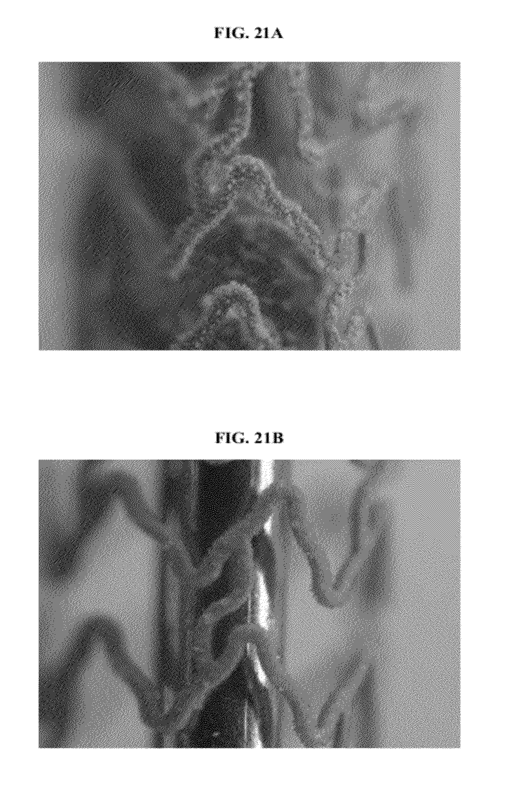

As shown in FIG. 21A, a highly uniform deposition of drug was observed on the stent surface after 30 minutes of deposition while cooling.

As shown in FIG. 21B, the stent surface was completely covered with rapamycin following an additional 2 hours of incubation at room temperature.

As shown in FIGS. 22A and 22B, CrCo stents subjected to rapamycin deposition for 30 minutes with cooling followed by 2 hours at room temperature resulted in coverage similar to that of stainless steel stents treated in the same manner (see, FIG. 21B).

A piece of a deposited rapamycin from one of the stainless steel stents was broken and the inner surface (i.e., the surface which faces the stent) was examined by SEM.

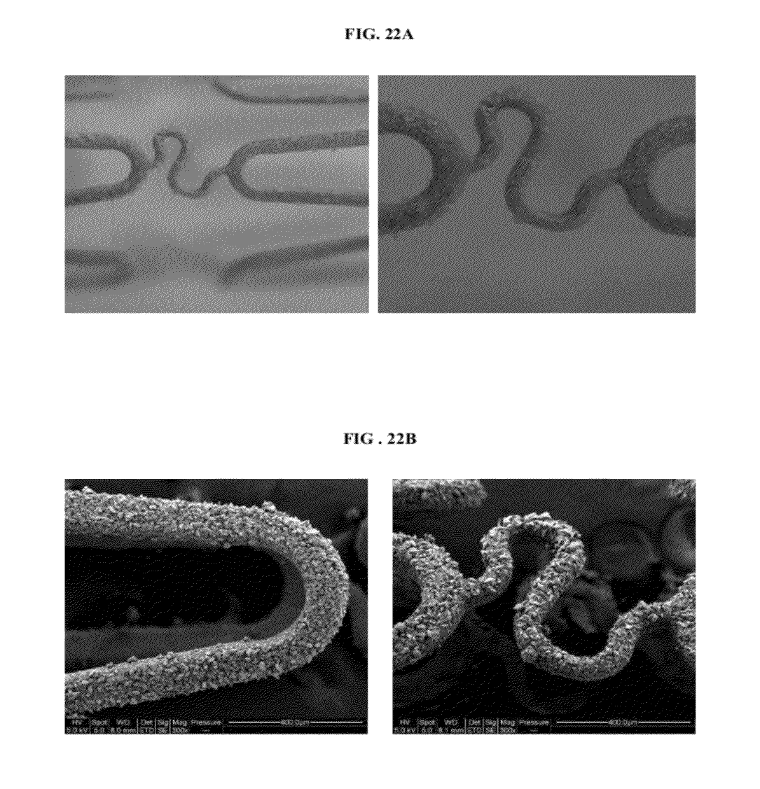

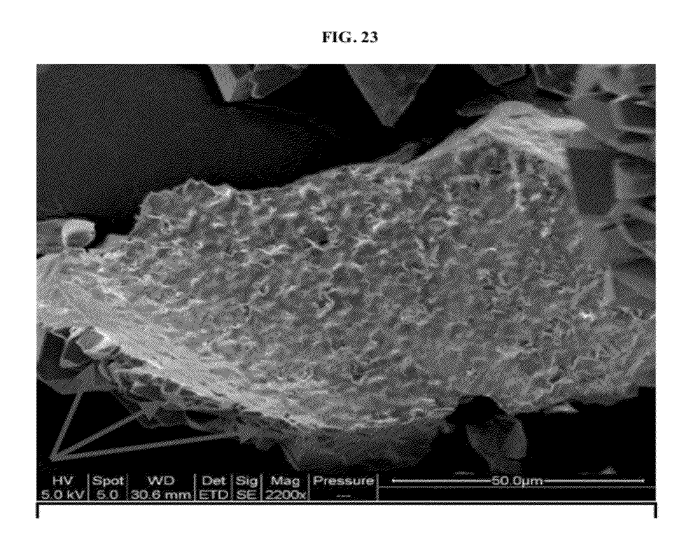

FIG. 23 presents the obtained SEM image, in which the primary layer of rapamycin, which serves as the base for continuous crystal growth, was observed. This image also indicates growth from the stent porous surface and outwards, as indicated by the observed impressions which match the porous surface of the bare stent (see FIG. 20). Crystals growing out of the surface are also observed (indicated by arrows).

The effect of drug solvation on crystal growth on stainless steel stents was examined. Solvation of rapamycin was increased by using 2 ml of solvent (ethyl acetate) in the solution instead of 1 ml.

As shown in FIGS. 24A and 24B, the rapamycin coating is smoother and appears more uniform, indicating smaller crystal size, when the stent was in a solution with 2 ml solvent (FIG. 24B) as compared with the coating obtained with 1 ml solvent (FIG. 24A). These results indicate that slower crystal growth occurred on the stent surface when more solvent was present.

The effect of cooling on crystallization was examined by repeating the above process without cooling the stent for 30 minutes.

Seeding was performed via sonication for 15 minutes in a solution of rapamycin (as received) that had been ground, and the stent was incubated overnight in a deposition solution (prepared as described hereinabove) at room temperature.

As shown in FIGS. 25A-25C, non-uniform deposition of rapamycin was observed with rapamycin-free areas on the surface of stainless steel stents.

In addition, shedding of crystals from the stent was observed, a phenomenon which was not observed when the stent was cooled during crystallization.

These results therefore indicate that the initial cooling process facilitates both crystal growth on the stent and attachment of the crystals to the stent surface.

The effect of seeding on crystallization was examined by repeating the above process without seeding the stent surface before crystallization. CrCo stents were examined in addition to stainless steel stents.

Crystallization was conducted by incubating the stents in a solution of 1, 10 or 25 mg rapamycin in 1 ml ethyl acetate and 20 ml n-hexane at room temperature for 72 hours.

No deposition of rapamycin was observed on the surface of stents immersed in a solution containing 1 mg rapamycin. In contrast, massive crystals, originating directly from the stent surface, appeared on the surface of stents incubated in the solutions containing 10 or 25 mg rapamycin.

As shown in FIG. 26, stainless steel stents incubated in a solution of 25 mg rapamycin in 1 ml ethyl acetate and 20 ml hexane were covered in a non-uniform manner with large crystals.

As shown in FIGS. 27A and 27B, similar results were obtained on CrCo stents incubated in a solution of 25 mg rapamycin in 1 ml ethyl acetate and 20 ml hexane.

The crystals exhibited a cubic morphology and a size of approximately 100 μm. The non-uniform coverage by the crystals resulted in unpredictable coverage of the stent surface, and in some samples, very few crystals were observed.

These results suggest that in the absence of seeding, a lower density of nucleation sites on the stent surface is present, indicating that the seeding procedure described hereinabove effectively increases the density of nucleation sites, thereby resulting in a more complete coverage of the surface with rapamycin crystals.

The above results indicate the advantageous effect of the seeding and cooling stages on the crystallization process.

The initial seeding step affects the coverage of the stent with the drug. Without being bound to any particular theory, it is suggested that the small ground crystals populate the stent raw surface as nucleation agents for further crystals growth, thus increasing the number of crystals and enhancing coverage of the stent surface.

It is further suggested that during the cooling procedure, when the sent is cooled and immersed in a cooled solution, the high temperature difference between the solution (0° C.) and the coolant cooling the stent (−78° C.) creates a supercooled and supersaturated solution close to the stent surface. Thus, it is suggested that in the absence of cooling, the crystals on the stent are poorly attached to the surface, and therefore tend to fall.

### Example 6

### Stages of Crystallization on Stainless Steel Surface

Crystallization was performed without a stent. Rather, the crystallization was performed on the surface of the hollow stainless steel rods connected to pipes through which coolant flowed, in order to examine the crystallization on a cooled metal surface.

The rods were put in a seeding solution with a ratio of 1 mg rapamycin (as received) which was ground and dispersed in 100 μl n-hexane, and sonicated for 15 minutes. The rods were then subjected to crystallization as described in Example 5 with cooling for 30 minutes, followed by incubation in solution for a time period ranging from 30 minutes to 2 hours. The deposition was analyzed at various stages of the process by weighing the deposited rapamycin and by XRD analysis.

As shown in FIGS. 28A-28C, no deposition on the metal was visible after 30 minutes of incubation in the crystallization solution (FIG. 28A), whereas some deposition was visible following 1 hour of incubation (FIG. 28B), and thicker deposition was visible following 2 hours of incubation (FIG. 28C).

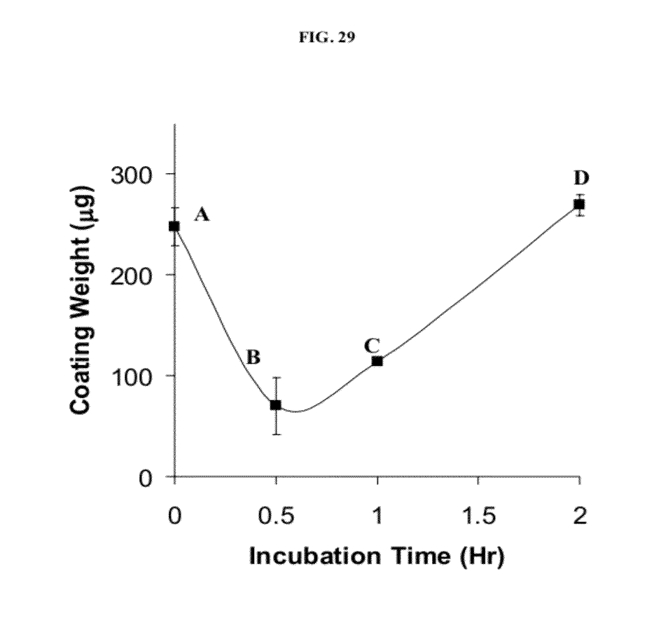

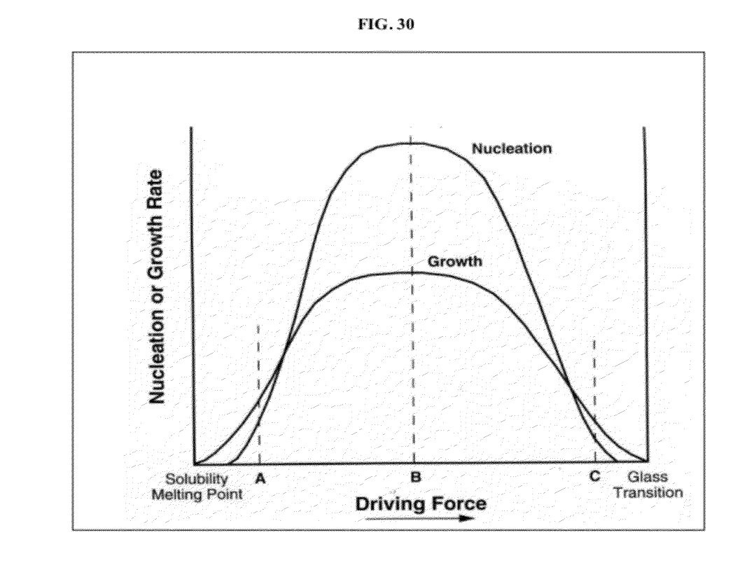

As shown in FIG. 29, the drug deposited after 30 minutes of cooling, which was shown by XRD analysis to be amorphous, almost completely disappeared during the first 30 minutes of incubation at room temperature. The amount of drug deposited on the rods then increased in an approximately linear manner for the duration of the incubation, forming a crystalline layer of rapamycin.

These results indicate that a crystallization process occurred on the cooled stainless steel rods, similar to the process observed on stents, due to the seeding of small rapamycin crystals attached to the metal surface during sonication (i.e., seeding), which serve as nucleation agents. The thickness of the crystalline layer was time-dependent as observed both visually and by weighing the deposited rapamycin.

The above results further demonstrate that amorphous rapamycin is formed when there is a strong crystallization driving force (i.e., during cooling). During incubation at room temperature, the amorphous rapamycin disappears and is replaced by more stable crystalline rapamycin. The crystallization that occurs during the incubation stage was enhanced by seeding the surface beforehand. In some cases when seeding is not performed, the amorphous form of rapamycin disappeared without being replaced by crystalline rapamycin (data not shown).

### Example 7

### Crystallization with a Lower Coolant Flow Rate and Seeding

Crystallization of rapamycin on stainless steel stents (Translumina) was performed as described in Process D in Example 3, except that 10 mg rapamycin was used instead of 3 mg. Deposition was conducted for a time period ranging from 4 minutes to 2 hours, followed by removal of the stent from solution and drying of the stent.

Seeding of the stent surface was performed via sonication of the stent for 15 minutes with a dispersion of 500 μg ground rapamycin crystals in 500 μl n-hexane.

This process is a representative crystallization process which occurs via conditions which lower deposition driving force, resulting in only a low deposition driving force, where the rate of nucleation is lower than the rate of crystal growth. The dependence of nucleation and crystal growth rates on deposition driving force is depicted schematically in FIG. 30. Conditions of low deposition driving force are depicted as region A in FIG. 30.

These conditions result in crystals that grow directly from the surface during the initial step in the crystallization process. Accordingly, no secondary incubation process is required to obtain a crystalline layer.

The effect of the time of crystallization (with cooling) on the weight of rapamycin coating deposited on the stent surface is shown in Table 1:

TABLE 1

| Cooling time | | Coating weight (μg) |
|---|---|---|
| 2 | hours | 470 |
| 1 | hour | 355 |
| 30 | minutes | 208 |
| 15 | minutes | 115 |
| 7.5 | minutes | 76 |
| 4 | minutes | 49 |

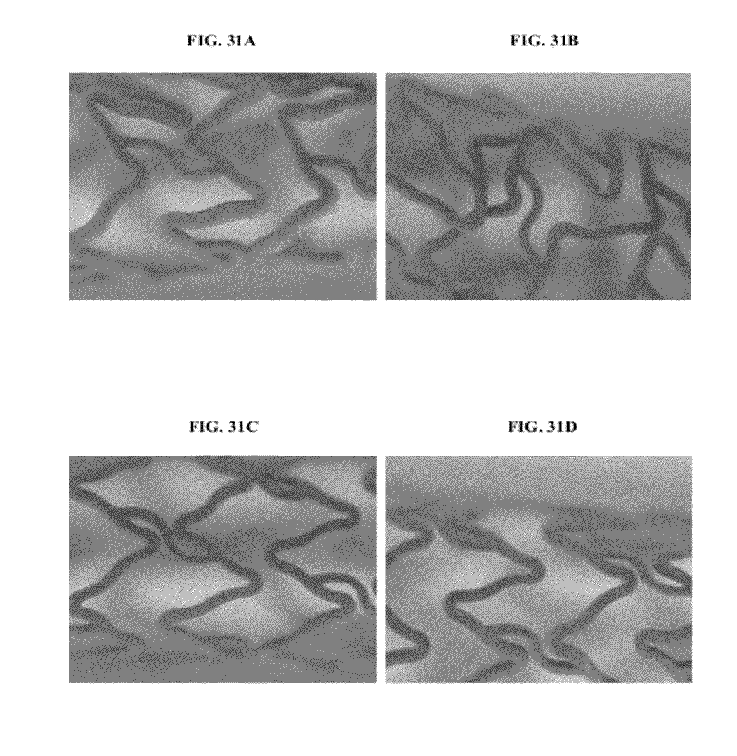

As shown in FIGS. 31A-31D, little crystallization was visible on the surface of the stent following 15 or 30 minutes of crystallization, whereas crystallization was clearly visible following 2 hours of crystallization.

The immediate effect of decreasing the flow rate was a temperature increase in the stent environment. The coolant runs in the system pipes slower, such that the hollow rod is cooled less effectively, resulting in a higher temperature than that obtained with higher coolant flow rates. A smaller deposition driving force is applied, which causes lower mass transfer to the stent surface. Without being bound to any particular theory, it is suggested that the crystalline state is kinetically favored at lower deposition driving forces, whereby the amorphous state is kinetically favored at high deposition driving forces. Consequently, nucleation on the stent surface is reduced, and a higher proportion of deposition occurs in the form of crystal growth.

### Example 8

### Increase of Crystalline Drug Over Time

The increases in the weight of deposited rapamycin obtained by different processes were examined.

Crystalline rapamycin was deposited on stents using constant cooling with a low deposition driving force, via Process D as described in Example 3, or by incubation at room temperature following cooling with a high deposition driving force, via Process C as described in Example 3.

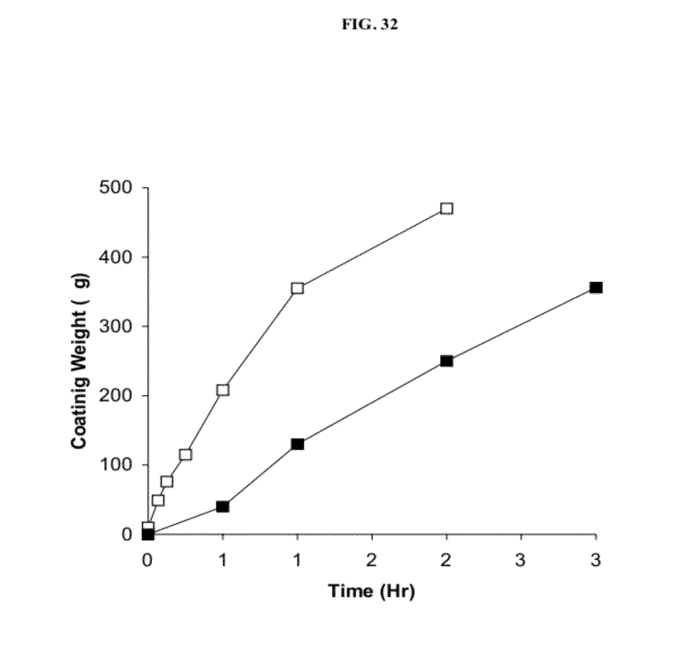

As shown in FIG. 32, the amount of deposited rapamycin obtained with either process increases in an approximately linear manner over time.

These results further indicate that the amount of deposited crystalline drug may be controlled by the time of crystallization.

### Example 9

### Formation of Crystals for Seeding

Instead of mechanical grinding, the following process was used to produce small rapamycin crystals.

0.25 grams of rapamycin were dissolved in 10 ml ethyl acetate and the solution was stirred for 30 seconds. 10 ml n-Hexane was added and the solution was stirred for additional 30 seconds to form a clear solution. The solution was evaporated in a round glass flask on an ice bath to form a thin coat of rapamycin on the flask surface. 50 ml of n-hexane was added and the solution was sonicated for 30 minutes at 0° C. A thin powder of rapamycin crystals was formed and the n-hexane was evaporated again at 0° C. 5 ml n-hexane was added and the solution was sonicated for 5 minutes and then evaporated at 0° C. The obtained dry powder was transferred to a 20 ml vial and sonicated for 20 minutes at room temperature. Homogeneous thin crystals of rapamycin were obtained, with a melting point of 186° C. The crystal size was determined by SEM to be approximately 500 nm. The size and homogeneity of the obtained crystals render such crystals particularly suitable for seeding.

The crystals were used in a seeding crystallization of stent surfaces by being sonicated with stents in n-hexane, as described in Example 5.

FIG. 33A shows a stainless steel stent (Johnson & Johnson) prior to seeding, and FIG. 33B shows the same surface after being seeded according to the above procedure. Rapamycin crystals are visible on the surface as white specks.

FIG. 34 shows SEM images of the rapamycin crystals seeded on the stent surface.

Crystalline rapamycin was then deposited on the stent surface, using the process described in Example 6.

As shown in FIG. 33C, the stent was covered by a highly uniform layer of small rapamycin crystals.

### Example 10

### Drug Release Profile of Amorphous and Crystalline Drug

The elution profiles of amorphous and crystalline rapamycin were compared, by measuring the pharmacokinetics by HPLC as described in the Methods section hereinabove.

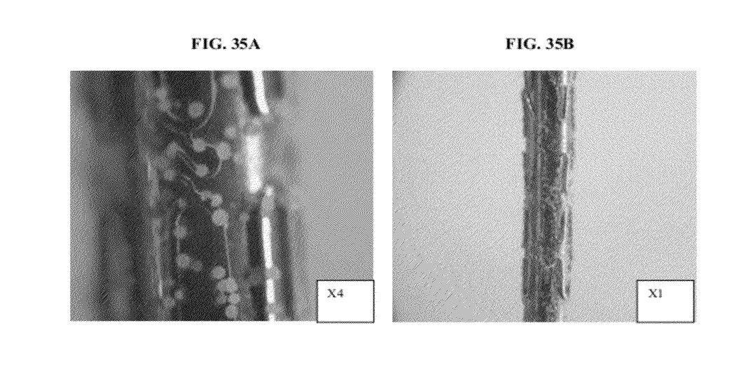

Deposition of crystalline rapamycin onto stents was performed according to a method similar to that of Process C described in Example 3. Stents were immersed in a solution of 25 mg rapamycin in 1 ml ethyl acetate and 20 ml n-hexane for 30 minutes while being cooled and were then left in the solution overnight at room temperature. FIG. 35 shows light microscope images of the obtained results.

Together with the stainless steel rod, stents were incubated in a pH 7.7 phosphate buffer for 5 days, at 37° C. During incubation, the crystals did not remain on the stents, but fell into the solution, remaining as crystals at the bottom of the vial. Samples were taken from 3 stents having 395, 537 and 612 μg rapamycin applied thereon, as described in the “methods” section hereinabove.

For comparison, 3 CrCo (L605) stents were spray-coated with amorphous rapamycin (at weights of 669, 645, 546 μg) and then were placed in a phosphate buffer, under the same conditions, for drug release measurements.

As shown in FIG. 36, the spray-coated amorphous rapamycin rapidly eluted from the stents, whereas the crystalline rapamycin had slower release. Within the first 24 hours, 55% of drug was released to the solution in the control samples, with very little release in the following days, whereas 44% of the drug was released from crystalline samples after 5 days, in an almost linear pattern.

### Example 11

### Drug Release Profile of Different Amorphous Forms of Drug

The elution profile of amorphous spray-coated rapamycin and amorphous rapamycin deposited on the surface of CrCo rods were compared.

CrCo rods, spray-coated with rapamycin were prepared using rapamycin dissolved in ethyl acetate, as described hereinabove. The obtained samples contained 727, 885 and 883 μg rapamycin.

Direct amorphous deposition of rapamycin on CrCo rods was performed using a procedure similar to Process A in Example 3, except that the deposition time was 100 minutes. The obtained samples contained 730, 832 and 902 μg rapamycin.

The rods were then incubated in a solution of phosphate buffer with 0.02% SDS. 2 ml of the incubation solution was sampled at each time interval, and the incubation solution was then replenished with 2 ml of fresh solution.

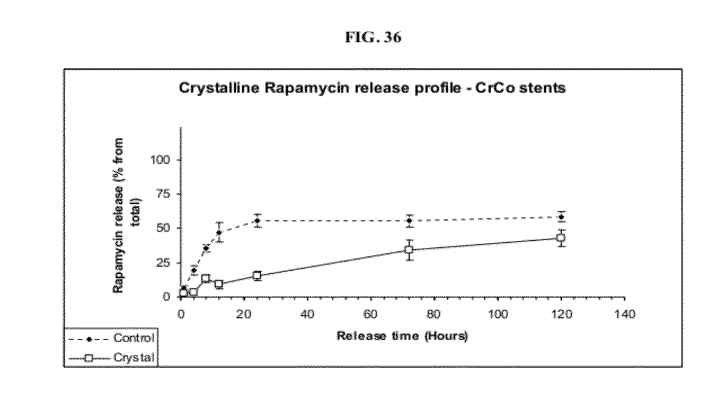

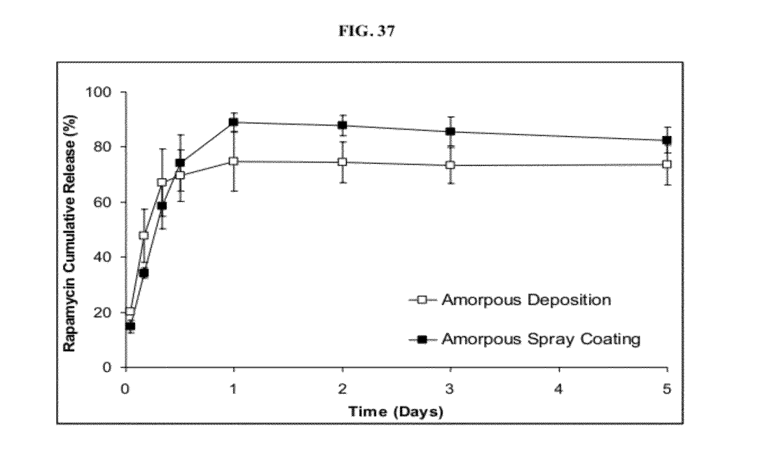

As shown in FIG. 37, the different amorphous forms of rapamycin had similar, relatively rapid, release rates.

These results support the findings by XRD analysis, which indicate that rapamycin deposited by the procedure described above is amorphous.

In addition, in view of the results of Example 10 hereinabove, these results suggest that all amorphous forms of deposited drug are released more rapidly than the crystalline deposited drug.

### Example 12

### Drug Release Profiles for Seeded Stainless Steel Surfaces

Crystalline rapamycin-coated stainless steel stents (Translumina) and rods, prepared by seeding and crystallization as described in Example 5, were incubated in phosphate buffer (pH 7.7) at 37° C. while shaking at 100 rotations per minute (RPM), as a model of physiological conditions, in order to obtain rapamycin elution profiles. Spray-coated stents (non-diazonium-electrocoated) and rods (electrocoated), with amounts of drug (in amorphous form) similar to that of the crystalline rapamycin-coated samples, were used as controls. 3 samples were measured for each test group and control group.

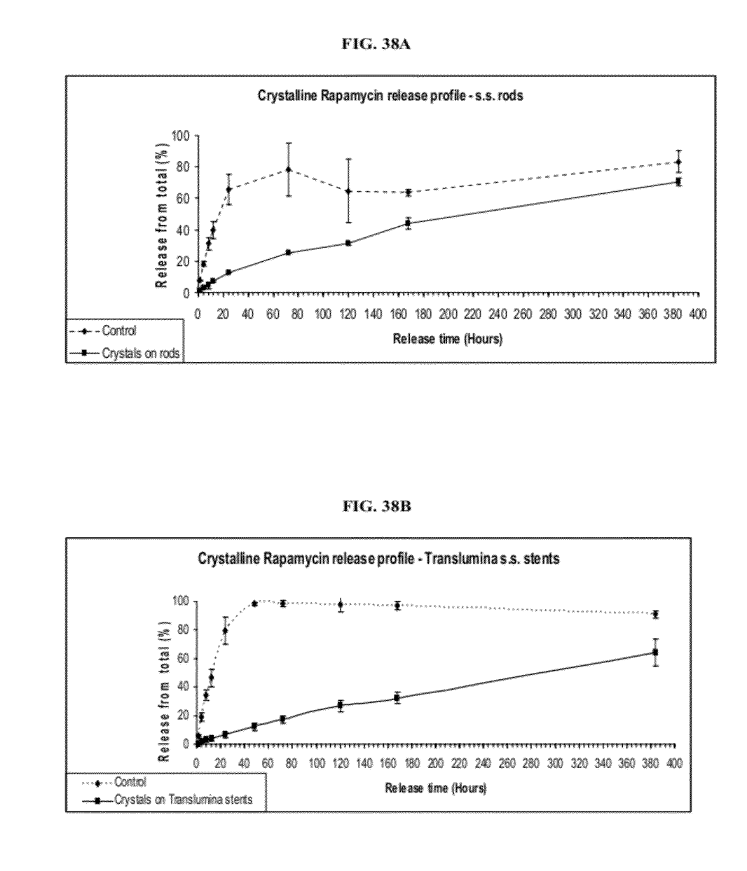

As shown in FIGS. 38A and 38B, a much slower elution profile was obtained in both stents and rods from crystalline-coated surfaces than from amorphous, spray-coated surfaces. After 16 days (384 hours), 70% of drug was eluted from crystalline-coated rods whereas 83% of drug was eluted from spray-coated rods. Rapamycin crystals on the stainless steel stents exhibited even slower elution: 64% drug was released within 16 days compared to 90-98% eluted from spray-coated stents (the apparent decreasing trend observed for spray-coated stents is due to deviation in detection caused by the high percentage of release).

Light microscopy images of crystalline rapamycin on a Translumina stainless steel stent were taken at several time points during the elution sampling.

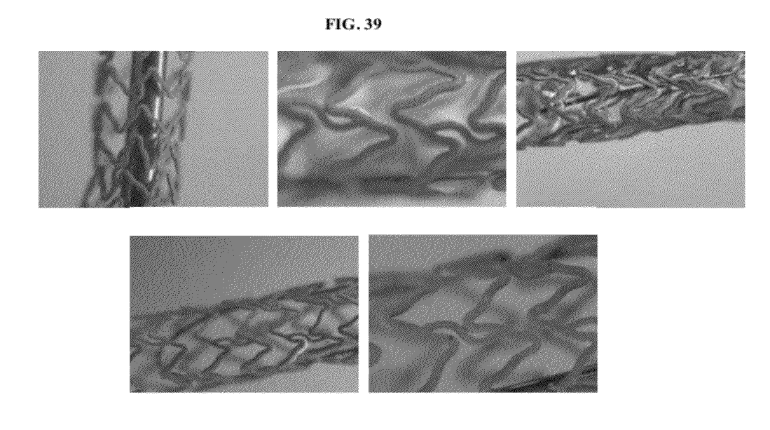

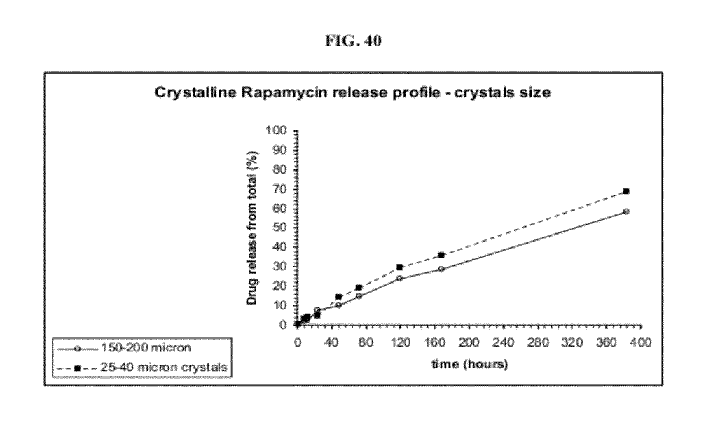

As shown in FIG. 39, the attachment of the crystals to the surface with time is clearly observable (some distortion in the images is present due to the presence of liquid on the stent surface). After 17 days the stents are fully covered with drug crystals.

Table 2 below presents the data obtained for the weight loss of rapamycin on the stent surface. Weight of rapamycin on stents with crystalline rapamycin coatings and on spray coated (control) stents was measured before (t=0) and after 17 days (t=17) of incubation at physiological conditions, as described hereinabove.

TABLE 2

| | | Rapamycin | |
|---|---|---|---|
| | Rapamycin (μg) | (μg) | |
| Stent type | t = 0 | t = 17 days | Weight loss (%) |
| Crystalline coating | 684 | 267 | 60% |
| Crystalline coating | 516 | 195 | 62% |
| Crystalline coating | 551 | 231 | 58% |
| Crystalline coating | 680 | 271 | 60% |
| Control | 580 | 4 | 99 |
| Control | 625 | 0 | 100% |
| Control | 536 | 10 | 98% |
| Control | 515 | 0 | 100% |

As shown in Table 2, in seeded stents coated with crystalline rapamycin, drug loss after 17 days of incubation at physiological conditions was approximately 60%. Both seeded and unseeded stents exhibited 60% loss. However, as described hereinabove, the seeding step had an effect on uniformity and quality of deposition.

The effect of crystal size on drug elution was also examined. Stents having rapamycin crystals of approximately 150-200 microns in diameter or of approximately 25-40 microns in diameter were prepared as follows:

Stents having rapamycin crystals in an average size in the range of 150-200 microns were obtained as described hereinabove, while applying a high deposition driving force (10 ml/minute), without seeding. Crystallization was conducted by immersing cooled stents in a cooled solution of 10 mg rapamycin in 1 ml ethyl acetate and 20 ml n-hexane, for 30 minutes, followed by incubation at room temperature for 2 hours.

Stents having rapamycin crystals in an average size in the range of 25-40 microns were obtained as described hereinabove, while applying a high deposition driving force (10 ml/minute), with seeding. Crystallization was conducted by immersing cooled stents in a cooled solution of 10 mg rapamycin in 1 ml ethyl acetate and 20 ml n-hexane, for 30 minutes, followed by incubation at room temperature for 2 hours.

Rapamycin release was determined as described hereinabove.

As shown in FIG. 40, the presence of larger crystals (150-200 microns) resulted in a slower drug elution profile.

### Example 13

### Drug Release Profiles for Seeded CrCo Stent Surfaces

The release profile of crystalline rapamycin from CrCo stents was compared to that of stents spray-coated with rapamycin.

Stents were seeded by being sonicated in 1 ml hexane with 300 μg ground rapamycin.

Stents with crystalline rapamycin deposition were prepared according to a variation of the static deposition process described in Example 2 hereinabove, using a solution of 3 mg rapamycin in 1 ml ethyl acetate and 20 ml hexane, and cooling the stent in solution for 80 minutes with a coolant flow rate of 6 ml/minute.

Spray-coated stents were prepared as described hereinabove, as controls.

Determination of drug release by incubation in phosphate buffer was performed as described in Example 12 hereinabove.

Stents were examined both with and without expansion of the stent via balloon catheter prior to incubation.

The obtained samples contained 69, 68, 87 and 89 μg of crystalline rapamycin on the non-expanded stents, and 62, 72 and 70 μg of crystalline rapamycin on the expanded stents.

The obtained control samples contained 70, 70, 70 and 73 μg of amorphous rapamycin on the non-expanded stents, and 72, 70, 70 and 71 μg of amorphous rapamycin on the expanded stents.

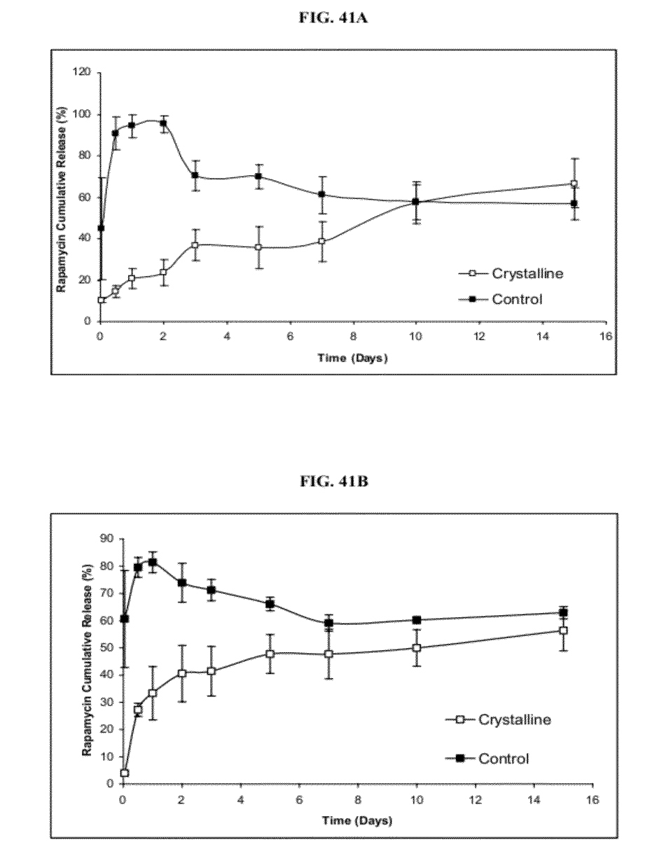

As shown in FIGS. 41A and 41B, crystalline rapamycin was released at a considerable lower rate than amorphous spray-coated rapamycin.

In addition, expansion of the stents increased the rate of release of both amorphous and crystalline rapamycin.

### Example 14

### Drug Release Profiles of CYPHER® Stent and Stent with Crystalline Drug

The release profile of rapamycin from seeded CrCo stents with crystalline rapamycin deposited thereon was compared to the release profile of rapamycin from CYPHER® stents (Johnson & Johnson). CYPHER® stents comprise a layer of spray-coated amorphous rapamycin in a polymer carrier covered by a polymeric top coat which slows the release of the rapamycin.

9 mm long CrCo stents were seeded by being sonicated for 15 minutes in 4 ml hexane with 1600 μg ground rapamycin. Crystalline rapamycin was then deposited on the surface of the stent as described in Example 13 hereinabove.

The amount of rapamycin on the CrCo stents was 61, 58 and 87 μg. CYPHER® stents, which are twice as long (18 mm) as the CrCo stents, contain 150 μg rapamycin.

Each stent was placed in a porous poly(tetrafluoroethylene) sleeve and then expanded via balloon catheter (using a pressure of 10 atmospheres) such that the stent filled the inner space of the sleeve. This procedure is designed to provide physical conditions similar to those present when expanding a stent in a blood vessel.

The sleeve with the stent was then placed in 4.5 ml incubation solution (phosphate buffer with 0.02% SDS). At each time interval, the incubation solution was removed in order to measure released rapamycin, and the sleeve was placed in a new vial with 4.5 ml fresh incubation solution. The amount of released drug at each time interval was calculated by summing each measured amount of released drug up to that time interval.

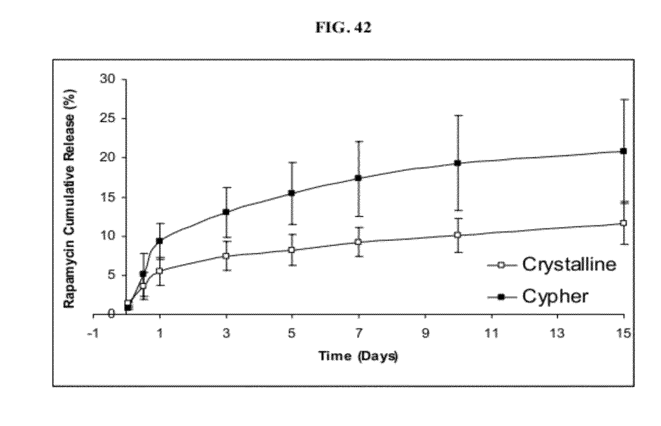

As shown in FIG. 42, the stents with crystalline rapamycin released rapamycin more gradually than the CYPHER® stent.

These results indicate that the use of crystalline drug can be at least as effective as the use of a polymeric carrier and a polymeric top coat in controlling drug release, whereby in the crystalline drug-coating described herein, the adverse effects associated with biostable polymers are avoided.

### Example 15

### Crystalline Drug with a Top Coat

Electrocoating, seeding and crystallization of CrCo stents was performed as described in Example 5.

Following crystallization, stents were spray coated at a rate of 0.025 ml/minute with a solution of 80 mg sodium carboxymethyl cellulose in 8 ml water, using a Sono-Tek syringe pump. The sodium carboxymethyl cellulose formed a smooth hydrogel layer which covered the crystalline drug. The hydrogel is water-soluble and consequently temporary. Approximately 85% of the hydrogel dissolved in water after 1 hour (data not shown).

The hydrogel can therefore be utilized for reducing friction when inserting a stent in a blood vessel, and thus serve as a lubricant, and for protecting the crystalline drug coating on the stent. As the hydrogel rapidly dissolves in an aqueous environment, it is unlikely to have an effect on the stent after the stent has been implanted.

In addition, the hydrogel top coat was shown to have a protective effect during expansion of the stent, as observed on stents with and without hydrogel lubricants expanded by a balloon catheter.

As shown in FIGS. 43A-43D, stents without a top coat exhibited some cracking of the rapamycin coating after expansion. In contrast, as shown in FIGS. 44A-44D, no cracking of the rapamycin coating were observed in stents with a top coat.

Loss of drug as a result of stent expansion was also characterized by weighing the samples to determine the weight of rapamycin thereon. Table 3 below presents the data obtained for the weight loss (in μg) of crystalline rapamycin in stents with and without a top coat.

As shown in Table 3, weight loss as a result of stent expansion was observed for stents without the lubricant, whereas no weight loss was observed for stents with the lubricant.

TABLE 3

| | | Lubricant | Weight loss after |
|---|---|---|---|
| Stent | Crystal weight | weight | Expansion |
| Sample 2 | 465 | NULL | 78 |
| Sample 3 | 446 | 97 | 0 |
| Sample 4 | 344 | 142 | 0 |
| Sample 6 | 513 | NULL | 154 |

These results indicate that a coating of crystalline drug on a stent may be protected from mechanical forces during expansion and insertion of a stent by a temporary, water-soluble top coat which covers the drug. Such a use of a top coat is particularly useful when the inner surface of the stent is covered with crystalline drug, as the inner surface is directly exposed to the mechanical forces applied by the catheter during expansion and insertion of the stent.

### Example 16

### Crystalline Drug Deposited on Non-Electrocoated Surface

Crystalline rapamycin was deposited on CrCo stents using the procedure described in Example 6 hereinabove, while applying a medium deposition driving force. The stents were not electrocoated beforehand.

As shown in FIG. 45, the stent was covered with a uniform layer of rapamycin crystals. 55 μg of rapamycin was deposited on the stent.

Similar results were obtained while using high deposition driving force.

These results indicate that deposition of a crystalline drug can be performed also without electrocoating of the stent's surface before deposition.

As shown in FIG. 55, the release profile of crystalline rapamycin from the non-electrocoated stent, as described herein, was similar to that obtained for electrocoated stents.

### Example 17

### Effect of Deposition Driving Force on Adherence of Crystals to Stent

The effects of different degrees of deposition driving force were compared. Stronger driving force was obtained using higher degrees of cooling and higher concentrations of rapamycin, as well as longer crystallization times.

Adherence of crystals to the stent was tested by expanding the stent by inflating a cardiovascular balloon, and then examining the surface of the stent.

Crystallization was performed in a similar manner to that described in Example 7 hereinabove, except that CrCo (L605) stents were used, rather than stainless steel stents. Seeding was conducted by sonicating the stent for 15 minutes in 500 μL n-hexane with 500 μg ground rapamycin. Crystallization was then performed using the static system for deposition described hereinabove in Example 2.

Samples exposed to moderate deposition driving forces were provided by using a coolant flow rate of 5 ml/minute during deposition, and concentrations of 2.5 or 7.5 mg rapamycin in 1 ml ethyl acetate with 20 ml hexane. The stent was then removed from the deposition system and dried at room temperature.

Samples exposed to strong deposition driving forces were provided by using a coolant flow rate of 10 ml/minute, and a concentration of 15 mg of rapamycin in 1 ml ethyl acetate with 20 ml hexane. The stent was then placed on a new rod and incubated in the crystallization solution for 2 hours at room temperature, and then removed and dried.

As shown in FIGS. 46A and 46B, moderate deposition driving forces resulted in a non-continuous layer of crystals which originate mainly from the surface of the stent.

As shown in FIGS. 46C and 46D, strong deposition driving forces resulted in a continuous layer of crystals, many of which do not originate from the stent surface. Substantial lateral crystal growth, originating from secondary nucleation, is evident.

As shown in FIGS. 47A and 47B, a non-continuous layer of crystals remained intact following expansion of the stent.

In contrast, as shown in FIGS. 47C and 47D, some portions of a continuous layer of crystals fell off the stent surface as a result of expansion of the stent.

These results indicate that limiting the driving force for deposition during the crystallization process can strengthen the adherence of the obtained crystals to the surface.

### Example 18

### Seeding with Upper Layer of a Crystalline Dispersion

10 mg of rapamycin were ground and dispersed in 1 ml of hexane. The solution was sonicated for 15 minutes to generate the dispersion. The dispersion was allowed to rest for 5 minutes and the upper 0.5 ml was redrawn to another vial. The dispersion had a milky color with a narrower distribution of particles than the original dispersion. The dispersion was used to dip-coat the stent several times until 5 μg of rapamycin adhered to the stent (as determined by weighing the stent before and after seeding).

Following seeding, crystallization was conducted using conditions having a moderate crystallization driving force, as described in Example 17 hereinabove. For comparison, other stents were seeded as described in Example 17, and subjected to the aforementioned crystallization procedure.

As shown in FIGS. 48A and 48B, the upper layer seeding described herein (FIG. 48A) resulted in a lower density of rapamycin, with less continuity, than that formed using the seeding procedure described in Example 17 (FIG. 48B).

### Example 19

### Seeding with Solution of Nanocrystals

A seeding solution is prepared with 2 mg/ml dissolved rapamycin in ethyl acetate and addition of 20 ml n-hexane according to the crystallization solution preparation procedure described in Example 17 hereinabove.

4 ml of this solution is taken, and stirred vigorously for 30 minutes at 0° C., thereby forming nanocrystals (seeds) of rapamycin. The stent is then immersed in the solution for 10 minutes, at 0° C., with stirring, so that the seeds become attached to its surface. Surface crystallization is then conducted using conditions having a moderate deposition driving force, as described in Example 17.

The initial seeding process results with stents that are coated with nanoparticles of rapamycin crystals with a diameter of approximately 100 nm. Further surface crystallization results in crystals which originate mainly from the surface, and with limited lateral crystal growth.

### Example 20

### Masking Inner Face of Stent During Crystallization Using Expandable Tube

As crystalline drug may fall off the inner side of a stent during stent expansion, due to the force of an inflated balloon used to expand the stent, stents were coated with crystalline drug only on the outer side, in order to avoid undesired and non-controlled drug release upon stent expansion.

FIGS. 49A and 49B present images of the system used in these experiments. The stent was mounted on an expandable polymeric tube, which expanded as a result of contact with n-hexane, thereby filling the inner space of the stent and masking the inner surface of the stent. The polymeric tube was inserted onto the metallic tube through which the coolant flowed (see, FIG. 49A).

The expandable polymeric tube utilized is made of a cross-linked rubber that expands upon absorbing hexane. Incubation of a polymeric tube having a diameter of 1.2 mm in hexane, for 5 minutes, results in a tube having a diameter of 1.4 mm.

Seeding was performed after the stent had been mounted on both the polymeric and metallic tubes (see, FIG. 49B). This methodology of seeding prevented seeding in the internal surface area of the stent.

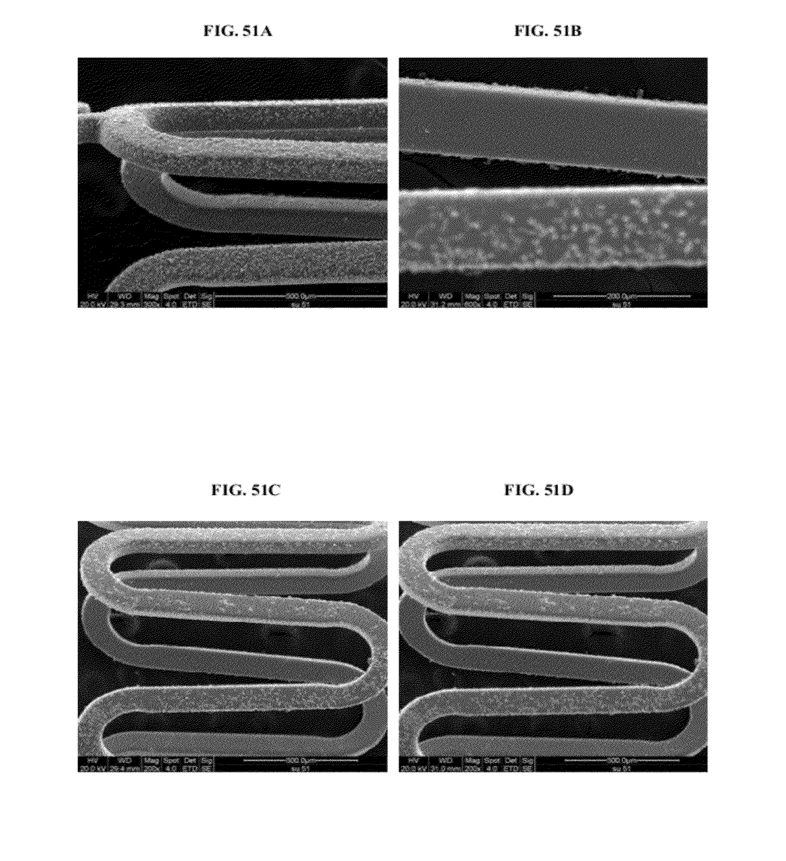

Crystallization was conducted similar to the procedure described in Example 17 using 2.5 mg rapamycin, except that the stent was mounted in the aforementioned manner, coolant flow rate was 6 ml/minute, and cooling was performed for 80 minutes. 51 μg drug was obtained on the stent. As shown in FIGS. 50A-50D and 51A-51D, the external side of the stent was coated with rapamycin crystals, whereas the internal side remained uncoated.

### Example 21

### Masking Regions of Stent During Crystallization Using Water-Soluble Polymer

The masking was performed on stainless steel tubes.

The tubes were seeded as described in Example 17 hereinabove. The tubes were then coated in certain regions with a solution of 20 mg/ml sodium carboxymethyl cellulose (CMC) in double distilled water (DDW), using a very thin paintbrush. Crystallization was then conducted for 30 minutes using a coolant flow rate of 10 ml/minute, followed by 2 hours incubation at room temperature, as described in Example 17.

Following crystallization, the tubes were washed for 15 minutes in DDW at 37° C., and then for 15 minutes at 60° C., to remove the water-soluble CMC.

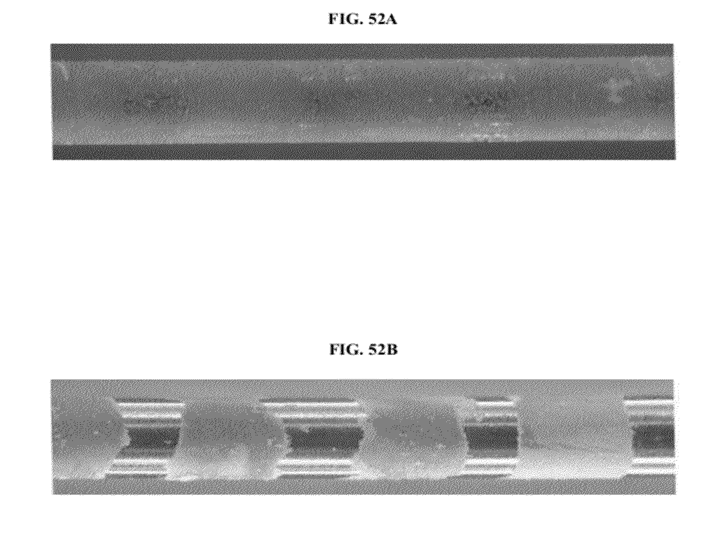

As shown in FIG. 52A, crystallization occurred at least to some extent over the whole surface of the tube.

However, as shown in FIG. 52B, regions masked with CMC became entirely free of crystals following the abovementioned washing procedure, whereas the unmasked regions remained coated with crystals.

These results demonstrate that the surface can be selectively coated with crystalline drug by masking portions of the surface with a polymer.

These results further suggest that masking with CMC may be used to prevent coating of the inner side of a stent with drug.

Thus, in additional experiments, the inner side of a stent was coated with CMC, subjected to crystallization and then washed, using the procedure described hereinabove. Results (data not shown) were similar to those described in Example 20.

### Example 22

### Seeding Limited to the External Surface of the Stent

The internal side of the stent is temporally covered during the seeding step by a physical barrier in a way that seeding is limited to the external surface of the stent. Temporary blockage of the internal side of the stent is done by putting the stent on an inflated balloon during the seeding step described in Example 17 hereinabove. For rapamycin, a crystalline coating is formed as described in Example 17. At the end of the process, the stent is coated mainly on its outer surface.

The abovementioned process may be performed on a stent which is already crimped on a balloon, wherein the balloon physically blocks its internal surface.

### Example 23

### Effect of Seeding on Crystallization Density with Moderate Deposition Driving Forces

Crystallization of rapamycin under conditions having a moderate deposition driving force was conducted as described in Example 17 hereinabove, except that the seeding procedure was not performed.

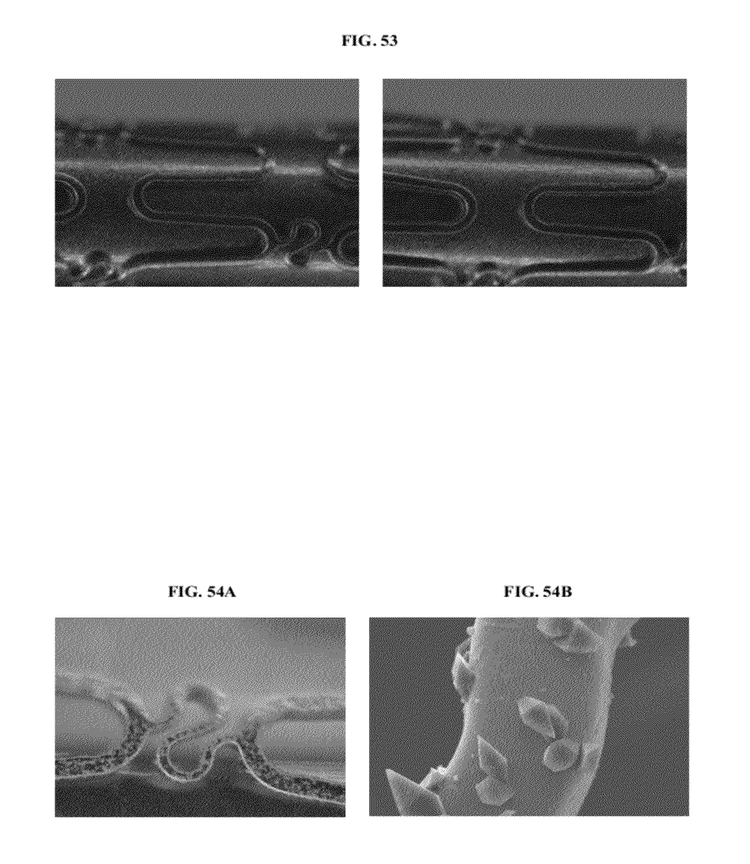

As shown in FIG. 53, the stent remained almost completely bare in the absence of seeding.

In contrast, as described in Example 17 and shown in FIG. 46B, the same procedure with seeding resulted in considerable deposition of crystalline drug on the stent surface.

These results indicate that the seeding stage has a beneficial effect in inducing crystallization on the surface using moderate deposition driving forces. These results are comparable to the results presented in Example 5 hereinabove, in which it was shown that the number of crystals and degree of surface coverage obtained with crystallization under conditions of high deposition force followed by room temperature incubation is reduced in the absence of seeding.

### Example 24

### Crystallization on a Polymer-Coated Stent

A DS-06-electrocoated stent was spray-coated with poly(D,L-lactic-co-glycolic acid) (intrinsic viscosity 0.6 deciliters/gram) to provide a 10 μg coating layer.

Crystallization was conducted under conditions having a moderate deposition driving force, as described in Example 17. Crystallization was performed using the polymer-coated stent.

As shown in FIGS. 54A and 54B, the stent was coated with crystals which originate mainly from the surface, with limited lateral crystal growth. As shown in FIG. 54B, the drug crystals appeared to be embedded in the polymeric layer.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-56. (canceled)

57. An article-of-manufacturing comprising an object having a surface and a therapeutically active agent being deposited onto at least a portion of said surface, at least 50% of said therapeutically active agent being in a crystalline form thereof, the article-of-manufacturing being devoid of a polymeric carrier for carrying said therapeutically active agent.

58. The article-of-manufacturing of claim 57, wherein said crystalline form of said therapeutically active agent is deposited directly onto said surface.

59. The article-of-manufacturing of claim 57, further comprising a base layer applied onto said surface, wherein said therapeutically active agent is being deposited onto said base layer.

60. The article-of-manufacturing of claim 59, wherein said surface is a conductive or semi-conductive surface and said base layer comprises at least one aryl moiety being electrochemically attached to said surface.

61. The article-of-manufacturing of claim 60, wherein said at least one aryl moiety is selected such that said base layer remains intact upon being subjected to physiological and/or mechanical conditions associated with said object for at least 30 days.

62. The article-of-manufacturing of claim 57, wherein at least 90% of said therapeutically active agent is in said crystalline form thereof.

63. The article-of-manufacturing of claim 57, further comprising a coat layer coating at least said portion of said surface having deposited thereon said therapeutically active agent.

64. The article-of manufacturing of claim 57, wherein said therapeutically active agent is selected from the group consisting of an anti-restenosis agent, an anti-thrombogenic agent, an anti-platelet agent, an anti-coagulant, a statin, a toxin, an antimicrobial agent, an analgesic, an anti-metabolic agent, a vasoactive agent, a vasodilator, a prostaglandin, a thrombin inhibitor, a vitamin, a cardiovascular agent, an antibiotic, a chemotherapeutic agent, an antioxidant, a phospholipid, an anti-proliferative agent, paclitaxel, rapamycin, and any combination thereof.

65. The article-of manufacturing of claim 57, wherein said therapeutically active agent is rapamycin.

66. The article-of-manufacturing of claim 57, wherein an amount of said therapeutically active agent that is released upon subjecting said object to physiological conditions for 24 hours is less than 20 percents by weight.

67. The article-of-manufacturing of claim 57, wherein said crystalline form of said therapeutically active agent comprises crystals having an average diameter in a range of from 2 to 200 microns.

68. The article-of-manufacturing of claim 57, wherein said object is a medical device.

69. The article-of-manufacturing of claim 68, wherein said object is an implantable medical device.

70. The article-of-manufacturing of any of claim 68, further comprising a packaging material, packaging said object and being identified, in or on said packaging material, for use in the treatment of a medical condition treatable by said medical device, wherein said medical condition is selected from the group consisting of a cardiovascular disease, atherosclerosis, thrombosis, stenosis, restenosis, a cardiologic disease, a peripheral vascular disease, an orthopedic condition, a proliferative disease, an infectious disease, a transplantation-related disease, a degenerative disease, a cerebrovascular disease, a gastrointestinal disease, a hepatic disease, a neurological disease, an autoimmune disease, and an implant-related disease.

71. An article-of-manufacturing comprising a stent having deposited, at least on a portion of a surface thereof, rapamycin, at least 90% of said rapamycin being in a crystalline form thereof.

72. A process of preparing the article-of-manufacturing of claim 57, the process comprising:
contacting a surface of said object with a solution containing said therapeutically active agent; and
cooling said surface to a temperature below a temperature of said solution, so as to form said crystalline form of said therapeutically active agent deposited on at least said portion of said surface.

73. The process of claim 72, further comprising seeding said surface with crystals of said therapeutically active agent prior to said contacting of said surface with said solution.

74. The process of claim 72, wherein said solution and said temperature are selected such that at least 50% of said therapeutically active agent is deposited on said surface in said crystalline form.

75. The process of claim 72, wherein when said solution and said temperature are selected such that at least a portion of said therapeutically active agent is deposited on said surface in a non-crystalline form, the process further comprises subsequently raising a temperature of said surface contacted with said solution, to thereby convert at least a portion of said non-crystalline form to said crystalline form.

76. The process of any of claim 72, wherein at least 90% of said therapeutically active agent on said surface is in said crystalline form.

77. The process of claim 72, wherein when said object further comprises a base layer applied onto at least a portion of said surface, the process further comprises, prior to contacting said surface with said solution of said therapeutically active agent, applying said base layer onto said surface.

78. The process of claim 77, wherein said surface is a conductive or semi-conductive surface, said layer comprises an aryl moiety and said applying comprises electrochemically attaching at least one aryl moiety substituted by at least one diazonium moiety to said surface.

79. A method of treating a subject having a medical condition in which implanting a medical device is beneficial, the method comprising:
implanting the medical device of claim 69 within said subject, thereby treating said medical condition.

80. An apparatus for performing the process of claim 72, the apparatus comprising;
a rod supporting said object;
a cooling mechanism being in thermal communication with said rod, for cooling said rod;
and
a receptacle for holding a solution comprising said therapeutically active agent, such that when said object is supported by said rod and said receptacle holds said solution comprising said therapeutically active agent, at least a portion of said surface of said object is in fluid communication with said solution comprising said therapeutically active agent.
